(19) United States
(12) Patent Application Publication
(10) Pub. No.: US 2026/0071252 A1
(43) Pub. Date: Mar. 12, 2026

(54) REVERSIBLE NANOPARTICLE AGGREGATES AND METHODS FOR DETECTING PROTEASES

(71) Applicant: The Regents of the University of California, Oakland, CA (US)

(72) Inventors: Jesse JOKERST, San Diego, CA (US); Maurice Gerard RETOUT, San Diego, CA (US); Wonjun YIM, San Diego, CA (US)

(21) Appl. No.: 19/107,123

(22) PCT Filed: Aug. 28, 2023
(86) PCT No.: PCT/US2023/031261

§ 371 Date: Feb. 27, 2025

Related U.S. Application Data
(60) Provisional application No. 63/401,914, filed on Aug. 29, 2022.

(51) Int. Cl. C12Q 1/37; B82Y 15/00; G01N 21/78

(52) U.S. Cl. CPC C12Q 1/37; G01N 21/78; B82Y 15/00; G01N 2333/9513

(57) ABSTRACT

In alternative embodiments, provided are compositions, including products of manufacture and kits, and methods, for detecting proteases including detecting proteases in a biological sample. In alternative embodiments, products of manufacture, formulations, mixtures or kits are used for stabilizing (or substantially stabilizing) nanoparticles in a reversible aggregate, and, detecting the presence of a protease in a fluid, wherein optionally the products formulations or mixtures can aggregate or assemble into nanoparticles such that the nanoparticles undergo plasmonic coupling, and the plasmonic coupling is reversible (or substantially reversible) leading to monodisperse nanoparticles when a chemical cue or signal is added.

### RELATED APPLICATIONS

This Patent Convention Treaty (PCT) International Application claims the benefit of priority under 35 U.S.C. § 119(e) of U.S. Provisional Patent Application Ser. No. 63/401,914, filed Aug. 29, 2022. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under AG065776, A1157957, DE031114; 1R21AI157957; 1R01DE031114, S10 OD 023527 awarded by the National Institutes of Health; and ECCS-1542148 and ECCS-2025752 awarded by the National Science Foundation. The government has certain rights in the invention.

### TECHNICAL FIELD

This invention generally relates to biochemistry. In alternative embodiments, provided are compositions, including products of manufacture and kits, and methods, for stabilizing nanoparticles and detecting proteases.

### BACKGROUND

Plasmonic nanoparticles are colloidally stable when the repulsive electrostatic and steric forces are balanced by the attractive Van der Waals forces. These forces vary as a function of separation, and stable colloids are at a local energy minimum. Salts and complex media can disrupt this energy minimum and induce aggregation—this aggregation is nearly always irreversible because the attractive Van der Waals forces scale to the sixth power. This aggregation leads to plasmonic coupling, which in turn leads to a color change. This plasmonic coupling has been extensively exploited to build compelling colorimetric sensors for the visual detection of biomarkers. However, most of the reported platforms suffer from (i) false positives and (ii) the incapacity to operate in complex matrices such as biological fluids. Most of the nanoparticles-based sensor use the aggregation of nanoparticles to cause a color change of the suspension. This work describes a way to reversible aggregate/assemble/couple plasmonic nanoparticles and to exploit this system for colorimetric protease detection.

### SUMMARY

In alternative embodiments, provided are products of manufacture, formulations, mixtures or kits for: stabilizing (or substantially stabilizing) nanoparticles in a reversible aggregate, and, detecting the presence of a protease in a fluid,
wherein optionally the products formulations or mixtures can aggregate or assemble into nanoparticles such that the nanoparticles undergo plasmonic coupling, and the plasmonic coupling is reversible (or substantially reversible) leading to monodisperse nanoparticles when a chemical cue or signal is added,
the products of manufacture, formulations, mixtures or kits comprising:
(a) a compound X capable of making or forming into reversibly aggregated (or substantially reversibly aggregated) nanoparticles (NPs),
wherein the compound X comprises:
a nanoparticle (NP) aggregated with or stabilized with (Arginine)ₓ (or Argₓ or Rₓ), or an Argₓ citrate-stabilized nanoparticle (citrate stabilizes the NPs until the peptide aggregates them), or a plurality of Argₓ citrate-stabilized nanoparticles (NPs), or Argₓ-NPs, wherein x is an integer 2, 3, 4, 5 or 6, and optionally the Rₓ comprises AA, AAA, AAAA (SEQ ID NO:1), AAAAA (SEQ ID NO:2) or AAAAAA (SEQ ID NO:3), or optionally the NP comprises a di-arginine (Arg) citrate-stabilized nanoparticle, or a plurality of di-arginine (Arg) citrate-stabilized nanoparticles (NPs), or Arg-Arg-NPs,
a nanoparticle (NP) aggregated with or stabilized with Lysineₓ-Rₓ (or -Lysₓ-Rₓ, or Kₓ-Rₓ, or an Kₓ-Rₓ citrate-stabilized nanoparticle, or a plurality of Kₓ-Rₓ citrate-stabilized nanoparticles (NPs), or Kₓ-Rₓ-NPs, wherein x is an integer 1, 2, 3, 4, 5 or 6, and optionally the Kₓ-Rₓ comprises KR, RK, RRK, KRR, RKR, RRKR (SEQ ID NO:4), RKRR (SEQ ID NO:5), KRRR (SEQ ID NO:6), RRRK (SEQ ID NO:7), KKRR (SEQ ID NO:8), KKKR (SEQ ID NO:9), RKKK (SEQ ID NO:10), KRRK (SEQ ID NO:11) or RKKR (SEQ ID NO:12),
and compound X aggregates (or is capable of aggregating, or substantially aggregates) when in a liquid solution, and optionally the liquid solution comprises (a) a biological fluid, optionally a biological fluid from an in vivo source, optionally an undiluted and/or untreated biological fluid from an in vivo source, and optionally the biological fluid from an in vivo source comprises blood, plasma, saliva, urine, bile, a lacrimal duct solution (a tear), or cerebrospinal fluid (CSF); (b) a cell lysate; or (c) water, distilled water, saline or sea water,
wherein optionally compound X, or the citrate-stabilized compound X, or the citrate-stabilized nanoparticle (NP) comprises or is conjugated to a metal to generate a metal-nanoparticle or metal-compound X, and optionally the metal of the metal nanoparticle or metal-compound X comprises silver (Ag) (for example, the nanoparticle comprises Arg-Arg-Ag-NP) or gold (Au) (for example, the nanoparticle comprises Arg-Arg-Au-NP); and
(b) a compound Z conjugated to a poly(ethylene glycol) (Z-PEGₓ), or a plurality of poly(ethylene glycol) (Z-PEGₓs), wherein X is an integer 1, 2, 3, 4, 5, 6, 7, 8, 9, 10, 11, 12, 13, 14 or 15, or X is an integer between about 1 and 40, or between about 2 and 30, or compound Z is conjugated to a peptide,
wherein compound Z is conjugated to:
(i) a poly(ethylene glycol) (Z-PEGₓ), or a plurality of poly(ethylene glycol) (Z-PEGₓs) is capable of dissociating compound X aggregates (for example, Arg-Arg-NPs aggregates) in a liquid solution,
wherein each Z-PEGₓ is conjugated to a protease peptide substrate (or an amino acid sequence specifically recognized and cleaved by a protease) by a compound Y comprising a moiety or a functional group linking the Z-PEGₓ to the protease peptide substrate (or Z-PEGₓ-Y-protease peptide substrate), or
(ii) a peptide,
and optionally each peptide comprises two three four, five, six, seven, eight, nine, ten, eleven or twelve or more amino acids, and optionally the peptide comprises EEKKPPC (SEQ ID NO:18),
and optionally one way of coupling Z to the nanoparticle comprises use of thiol on a Cys moiety,
and optionally the peptide comprises one or more amino acids with a negative charge at the opposite end that is bound to the nanoparticle where one or more of these negative amino acids are acetylated to increase negative charge,
and optionally each peptide comprises one or more spacer amino acids including glycine, proline or alanine that increase the distance between the binding amino acid and the charged amino acid,
and optionally each peptide comprises EEKKPPC, where the cysteine (or C) binds to a gold (Au) surface, E (glutamic acid) has a negative charge and is acetylated, K (lysine) is positively charged, and P (proline) adds steric bulk,
wherein optionally the compound Z comprises:
   a thiol to generate a thiolated (HS) poly(ethylene glycol) (HS-PEGₓ), or a plurality of thiolated (HS) poly(ethylene glycol) (HS-PEGₓs),
   an alkyne, to generate a poly(ethylene glycol) (ALK-PEGₓ), or a plurality of poly(ethylene glycol) (ALK-PEGₓs),
   a lipoic acid group, to generate a poly(ethylene glycol) (LIP-PEGₓ), or a plurality of poly(ethylene glycol) (LIP-PEGₓs),
wherein optionally the compound Y comprises:
   a carboxyl group (for formation of an amide bond between the PEGₓ and peptide),
   an azido group (for formation of a copper (I)-catalyzed alkyne-azide cycloaddition (CuAAC) to chemically join the PEGₓ and peptide),
   an alkyne group (for formation of a copper (I)-catalyzed alkyne-azide cycloaddition (CuAAC) to chemically join the PEGₓ and peptide), or
   a maleimide group (to generate a Michael reaction addition thiol group to chemically join the PEGₓ and peptide).
and optionally each polyethylene glycol (PEG) moiety, or x, comprises one, two three four, five, six, seven, eight, nine, ten, eleven or twelve or more PEG repetitions,
wherein each Z-PEGₓ is conjugated to a protease peptide substrate (or an amino acid sequence specifically recognized and cleaved by a protease) (Z-PEGₓ-Y-peptide),
and a plurality of compound X make or form into reversibly aggregated nanoparticles (NPs) when in the liquid solution, and optionally can stay in a substantially aggregated state indefinitely before being resuspended back to a state of monodispersity by the addition of compound Z to the liquid solution.

In alternative embodiments of products of manufacture, formulations, mixtures or kits and provided herein:
the protease is a viral or a mammalian protease, and optionally the mammalian protease is a human protease, and optionally the viral protease is a coronavirus protease, and optionally the coronavirus protease is a Covid-19 protease or SARS-CoV-2 (Mpro);
the fluid comprises or is derived from: (a) a biological fluid, optionally a biological fluid from an in vivo source, optionally an undiluted and/or untreated biological fluid from an in vivo source, and optionally the biological fluid from an in vivo source comprises blood, plasma, saliva, urine, bile, a lacrimal duct solution (a tear), or cerebrospinal fluid (CSF); (b) a cell lysate; or (c) water, distilled water, saline or sea water;
the citrate-stabilized NPs is about 20 nm in diameter, or is between about 10 and 50 nm in diameter; and/or
the citrate-stabilized NPs is prepared using a Turkevich method comprising:
(a) rapidly injecting an aqueous solution of sodium citrate tribasic dihydrate (SCTD) (optionally 150 mg SCTD, 5 mL aqueous solution) into an aqueous solution of HAuCl₄·3H₂O (optionally 45 mg HAuCl₄·3H₂O, 300 mL aqueous solution) under boiling conditions and vigorous stirring, to produce a reaction mixture; and
(b) the reaction mixture is left boiling while stirring for another about 15 min and then cooled down to room temperature (RT) to generate a deep red dispersion, and the deep red dispersion was then purified by applying one round of centrifugation at about 18,000 g for about 30 min to generate a pellet of AuNPs-citrate, and the pink supernatant is discarded, and the resulting pellet of AuNPs-citrate is redispersed in deionized water by sonication and stored at ambient conditions.

In alternative embodiments, provided are solutions or formulations comprising the plurality of compound Xs (for example, a plurality of Arg-Arg-NPs) of claim 1(a) and the plurality of Z-PEGₓ-Y-peptides as provided herein, and optionally the solution or formulation comprises a saline solution, water, a cell lysate or a biological solution, and optionally the biological solution comprises a biological fluid from an in vivo source comprises a cell lysate solution, blood, plasma, saliva, urine, bile, a lacrimal duct solution (for example, a tear), or cerebrospinal fluid (CSF).

In alternative embodiments, provided are methods for detecting a protease in a sample, comprising: mixing the compound Xs (for example, a plurality of Arg-Arg-NPs) as provided herein and the Z-PEGₓ-Y-peptide as provided herein in a sample, and presence of a protease capable of specifically recognizing and cleaving the peptide is detected via a proteolytic cleavage that releases Z-PEGₓ fragments (from Z-PEG-Y-peptide), thus inducing the dissociation of the aggregated compound Xs (for example, AuNPs-citrate assemblies) resulting in a change in color of the solution (for example, turning the solution from blue to red if X is AuNPs-citrate).

In alternative embodiments of methods as provided herein: the sample comprises a cell lysate or a biological solution, and optionally the biological solution comprises a biological fluid from an in vivo source comprises a cell lysate solution, blood, plasma, a lacrimal duct solution (a tear), saliva, urine, bile or cerebrospinal fluid (CSF); or the sample comprises water or a saline solution.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-H illustrate peptide-induced assembly of AuNPs-citrate. (FIG. 1A-D) TEM images and multispectral advanced nanoparticle tracking analysis (MANTA)29 image and size distribution of AuNPs-citrate before:

FIG. 1A and FIG. 1C illustrate images of Au-NPs-citrate, and FIG. 1B and FIG. 1D illustrate 10 minutes after the addition of 10 μM of Arg-Arg; FIG. 1C and FIG. 1D illustrate MANTA records images of the particles' light scattering via three differently colored lasers (blue, green, and red); the scattering depends on the particle's size; MANTA counts the nanoparticles and calculates their size;

FIG. 1E graphically illustrates data showing modification of the optical properties of AuNPs-citrate during the assembly with Arg-Arg. Insets show the pictures of the samples for concentrations of Arg-Arg of 0, 1, 2, 5 and 10 μM;

FIG. 1F graphically illustrates data showing ratio of the absorbances Abs.520 nm/Abs.700 nm of AuNPs-citrate as a function of peptide concentration (including: amino acid residue R, peptide RR, peptide RRRRR (SEQ ID NO:15) and peptide RGGGR (SEQ ID NO:16));

FIG. 1G graphically illustrates data showing UV-Vis spectra of Arg-Arg-AuNPs 1 day or 120 days old; and

FIG. 1H illustrates image of pictures of Arg-Arg-AuNPs dried and resuspended in pure water after 5 seconds of sonication,
as discussed in further detail in Example 1, below.

FIG. 2A-E illustrate characterization of the dissociation of AuNPs assemblies with HS-PEG6-OCH₃ (“PEG” is polyethylene glycol); from left to right, TEM*, MANTA, ATR-FTIR**, and UV-Vis*** spectroscopy analysis of Arg-Arg-AuNPs (FIG. 2A) before or 10 minutes after the addition of (FIG. 2B) 2 μM, (FIG. 2C) 4 μM, (FIG. 2D) 8 μM, and (FIG. 2E) 12 μM of HS-PEG6-OCH₃; *all the images are at the same magnification; **all samples were cleaned by centrifugation from non-bound molecules (HS-PEGs, citrate or Arg-Arg) and the FTIR spectrum corresponds only to molecules attached to the gold surface; *** black dashed line shows the LSPR band of the initial AuNPs-citrate, as discussed in further detail in Example 1, below.

FIG. 3A-B illustrate dissociation efficiency of HS-PEG6-OCH₃ (“PEG” is polyethylene glycol) for alternative assembly conditions or coating ligands:

FIG. 3A illustrates dissociation efficiency of 100 μM of HS-PEG6-OCH₃ (“PEG” is polyethylene glycol) added to AuNPs-citrate assembled in peptide-free conditions; and

FIG. 3B illustrates dissociation efficiency of 100 μM of HS-PEG6-OCH₃ (“PEG” is polyethylene glycol) added to either AuNPs-citrate, AuNPs-BSPP, AuNPs-MBA, or AuNPs-S-PEG-COOH assembled with 10 μM of Arg-Arg or AuNPs-citrate assembled with 10 μM of Cys-Arg-Lys peptide,
as discussed in further detail in Example 1, below.

FIG. 4A-K graphically illustrate the effect of the PEG properties on the dissociation efficiency:

FIG. 4A graphically illustrates dissociation efficiency of X-PEG-OCH₃ (“PEG” is polyethylene glycol) molecules carrying various anchoring groups added to Arg-Arg-AuNPs. X=thiol (HS), hydroxy (OH), lipoic acid (LA) or Alkyne (Alk);

FIG. 4B graphically illustrates dissociation efficiency of HS-PEG4-OCH₃, MBA, or MPA added to Arg-Arg-AuNPs;

FIG. 4C graphically illustrates dissociation efficiency of HS-PEG226-OCH₃ compared to HS-PEG226-NH₂ (“PEG” is polyethylene glycol) added to Arg-Arg-AuNPs;

FIG. 4D graphically illustrates size of the assembly 20 minutes after the addition of HS-PEGₓ-OCH₃ (“PEG” is polyethylene glycol) to Arg-Arg-AuNPs of different molecular weight (20, 10, 5, 2, 1, 0.35 and 0.22 kDa); and

FIG. 4E-K graphically illustrate dissociation efficiency of HS-PEGₓ-OCH₃ (“PEG” is polyethylene glycol) concentration with different molecular weight added to Arg-Arg-AuNPs,
as discussed in further detail in Example 1, below.

FIG. 5A-D illustrate dissociation of AuNPs assemblies in complex matrices:

FIG. 5A illustrates dissociation efficiency of HS-PEG6-OCH₃ added to Arg-Arg-AuNPs suspended in various media; and

FIG. 5B illustrates the corresponding pictures before and 20 minutes after the addition of 10 μM of HS-PEG6-OCH₃ (“PEG” is polyethylene glycol); the complex medium was approximately 90% of the total volume except for the bile that was 20%;

FIG. 5C graphically illustrates dissociation of Arg-Arg-AuNPs with HS-PEG6-OCH₃ compared to the Arg-Arg-induced aggregation of AuNPs-citrate in various complex matrices, note that HEK/DMEM corresponds to HEK 293 cell lysates coming from suspension of 106 cells in Dulbecco's modified Eagle's media with 10% fetal bovine serum and 1% penicillin-streptomycin; and

FIG. 5D graphically illustrates UV-Vis spectra of dried Arg-Arg-AuNPs film after dissociation with aqueous solution of HS-PEG6-OCH₃ (“PEG” is polyethylene glycol) of various of concentrations. Insets show the absorbance at 520 nm as a function of the concentration of HS-PEG6-OCH₃ and the corresponding pictures,
as discussed in further detail in Example 1, below.

FIG. 6A-J illustrate trypsin sensing with HS-PEG-peptide (“PEG” is polyethylene glycol) conjugates (including peptides RRK, RRKRRK (SEQ ID NO:13), and RRKRRKRRK (SEQ ID NO:14):

FIG. 6A illustrates a scheme of the synthesis of HS-PEG-peptide (“PEG” is polyethylene glycol) conjugates via EDC/NHS chemistry including the three main sequences investigated as well as the size and charge of the compounds;

FIG. 6B-E illustrates and illustration of the sensing mechanism: the trypsin cleavage reduces the size and charge of the HS-PEG-peptide (“PEG” is polyethylene glycol) conjugate, which increases their capacity to dissociate the AuNPs assemblies, dissociation of Arg-Arg-AuNPs assemblies with either:

FIG. 6C, showing HS-PEG-RRK (where here “PEG” is not a peptide but is polyethylene glycol),

FIG. 6D, showing HS-PEG-RRKRRK (SEQ ID NO:13) (where here “PEG” is not a peptide but is polyethylene glycol), or

FIG. 6E showing HS-PEG-RRKRRKRRK (SEQ ID NO:14) (where here “PEG” is not a peptide but is polyethylene glycol), intact (black line) or digested by 1 μM of trypsin for 24 h at 37° C. (dashed blue line);

FIG. 6F illustrates dissociation of Arg-Arg-AuNPs assemblies with HS-PEG-TSG and HS-PEG-C12 either intact or digested by trypsin;

FIG. 6G illustrates trypsin detection using the three different HS-PEG-peptide conjugates and (H) the corresponding pictures;

FIG. 6I illustrates a picture of dried Arg-Arg-AuNPs assemblies after the addition of 50 μL of HS-PEG-RRKRRK (PEG is polyethylene glycol, the peptide is SEQ ID NO:13) incubated or not with 1 μM of trypsin; and

FIG. 6J illustrates comparison between the detection of 1 μM of trypsin spiked either in PBS or in urine as well as in PBS with 80 μM of AEBSF inhibitor (AEBSF is 4-(2-aminoethyl) benzenesulfonyl fluoride hydrochloride, a trypsin inhibitor), as discussed in further detail in Example 1, below.

FIG. 7A-C illustrates Mpro detection:

FIG. 7A graphically illustrates colorimetric signal obtained for the detection of the main protease of SARS-CoV-2 virus using HS-PEG-peptide conjugate to dissociate the AuNPs assemblies after the proteolytic cleavage;

FIG. 7B illustrated the structure of the exemplary HS-PEG that was conjugated to the peptide substrate specific to Mpro; and

FIG. 7C illustrate the structure of the HS-PEG-peptide that contains two HS-PEGs per peptide, note the peptide GTSAVLQSGFRK (SEQ ID NO:17).

FIG. 8A-D illustrates the dissociation of silver nanoparticles assemblies:

FIG. 8A illustrates pictures of silver nanoparticles samples dispersed in water then aggregated with di-arginine peptides then dissociated with HS-PEGs;

FIG. B graphically illustrates typical evolution of the UV-Vis spectrum of silver nanoparticles assemblies upon the addition of increasing concentrations of HS-PEGs; and

FIG. 8C-D illustrate: TEM images of silver nanoparticle assemblies (FIG. 8C) before or after (FIG. 8D) the addition of HS-PEGs.

FIG. 9A-H illustrates the dissociation of gold nanorods assemblies:

FIG. 9A illustrates an exemplary scheme of the assembly of gold nanorods with di-arginine peptide and the typical UV-Vis spectra, the data graphically illustrated in FIG. 9B, and corresponding TEM images illustrated in FIG. 9C;

FIG. 9D illustrates an exemplary scheme of the dissociation of assemblies of gold nanorods with HS-PEGs molecules and the typical UV-Vis spectra (FIG. 9E) and TEM images (FIG. 9F) that show the recovery of the optical properties and the dispersion of the particles;

FIG. 9G and FIG. 9H illustrate images showing the detection of trypsin activity in vivo (mouse model) with photoacoustic imaging using a HS-PEG-peptide conjugate (HS-PEG-RRKRRK) (PEG is polyethylene glycol, the peptide is SEQ ID NO:13) that can be cleaved by trypsin which dissociate the gold nanorods assemblies, leading to an increase of photoacoustic signal.

FIG. 10 illustrates TEM images of AuNPs-citrate at different magnifications, as indicated on the Figure.

FIG. 11 illustrates TEM images of Arg-Arg-AuNPs at different magnifications, as indicated on the Figure.

FIG. 12 illustrates the dissociation of an exemplary AuNPs assembly: TEM images of Arg-Arg-AuNPs after addition of 2 μM of HS-PEG6-OCH₃.

FIG. 13 illustrates the dissociation of an exemplary AuNPs assembly: TEM images of Arg-Arg-AuNPs after addition of 4 μM of HS-PEG6-OCH₃.

FIG. 14 graphically illustrates the dissociation of an exemplary AuNPs assembly: TEM images of Arg-Arg-AuNPs after addition of 8 μM of HS-PEG6-OCH₃.

FIG. 15 graphically illustrates the ratio of the absorbances at 520 nm and 700 nm of Arg-Arg-AuNPs dissociated with 10 μM of HS-PEG6-OCH₃ as a function of time between the formation of the assembly and the dissociation.

FIG. 16 graphically illustrates the dissociation efficiency of 10 μM of HS-PEG6-OCH₃ added to Arg-Arg-AuNPs in increasing NaCl concentrations.

FIG. 17 graphically illustrates the dissociation efficiency of 10 μM of HS-PEG6-OCH₃ added to Arg-Arg-AuNPs at different pH values.

FIG. 18 graphically illustrates the dissociation efficiency of 0, 5, 10 or 20 μM of HS-PEG6-OCH₃ added to Arg-Arg-AuNPs in increasing concentration of DTT.

FIG. 19 graphically illustrates the dissociation efficiency for Arg-Arg-AuNPs suspended in different complex matrices (plasma, water, urine, saliva, HEK) over time, where HEK indicates HEK cell lysates coming from suspension of 10⁶ cells/mL in DMEM medium.

FIG. 20 graphically illustrates the ratio of the absorbances at 520 nm over 700 nm of AuNPs-citrate suspended in various complex matrices, ratio of AuNPs-citrate dispersed in water=7.8.

FIG. 21 illustrates pictures of Arg-Arg-AuNPs suspended in saliva either pristine, after two hours or after two hours and addition of 10 μM of HSPEG₆-OCH₃. Red arrows show the formation of the macroscopic precipitates.

FIG. 22A-H illustrate pictures and UV-Vis spectra of dried Arg-Arg-AuNPs film solubilized via the addition of increasing concentrations of HS-PEG6-OCH3 in either (FIG. 22A) saliva, (FIG. 22B) plasma, (FIG. 22C) HEK cell lysates, (FIG. 22D) urine, the insets show the absorbance at 520 nm as a function of the HS-PEG6-OCH₃ concentration.

FIG. 23 graphically illustrates the dissociation efficiency of HS-PEG₁₂-COOH (634 Da) added to Arg-Arg-AuNPs.

FIG. 24A-B illustrates pictures of Arg-Arg-AuNPs 20 minutes after their exposition to 20 μM of HSPEG12-COOH that was initially mixed with the peptides RRK, RRKRRK (SEQ ID NO:13) or RRKRRKRRK (SEQ ID NO:14) either (FIG. 24A) in the presence or (FIG. 24B) the absence of EDC/NHS. In the absence of EDC/NHS, there is no conjugation between HS-PEG12-COOH and the peptide and thus, HS-PEG12-COOH can still dissociate the assemblies.

FIG. 25A-B illustrates matrix-assisted laser desorption/ionization (MALDI) analysis of HS-PEG-RRKRRK (SEQ ID NO:13) (the “PEG” here is not a peptide but is for polyethylene glycol); after the synthesis, the product was purified by HPLC and two compounds could be identified: (A) RRKRRK (SEQ ID NO:13) conjugated to one HS-PEG12-COOH and (B) RRKRRK (SEQ ID NO:13) conjugated to two HSPEG12-COOH units.

FIG. 26 graphically illustrates trypsin detection using HS-PEG-RRKRRK (SEQ ID NO:13) either in buffer (PBS 1×, pH 7.4) or in saliva.

FIG. 27 graphically illustrates a typical inhibition titration curve fitted with the Morrison equation for the competitive inhibitor, AEBSF (not a peptide, but rather is: 4-(2-Aminoethyl)-benzenesulfonyl fluoride, HCl). Inset shows the chemical structure of AEBSF and the Ki and IC₅₀ values. Briefly, 1 μM of trypsin was mixed for 30 minutes at room temperature with increasing concentration of AEBSF; then, HS-PEG-RRKRRK (SEQ ID NO:13) (the “PEG” here is not a peptide but is for polyethylene glycol) was added and the resulting solution was incubated at 37.5° C. for 2 hours before addition to Arg-Arg-AuNPs assemblies and the ratio of the absorbance at 520 nm and 700 nm was measured.

FIG. 28 illustrates images of dissociation study with 40 nm AuNPs: TEM images of AuNPs-citrate (40 nm) at different magnifications.

FIG. 29 illustrates images of dissociation study with 40 nm AuNPs: TEM images of Arg-Arg-AuNPs (40 nm) at different magnifications.

FIG. 30A-B illustrate images of size distribution of AuNPs-citrate (40 nm) (FIG. 30A) before and (FIG. 30B) 10 minutes after the addition of 2 μM of Arg-Arg obtained via multispectral advanced nanoparticles tracking analysis (MANTA).

FIG. 31A-L illustrates MANTA images and size histograms for Arg-Arg-AuNPs (40 nm) (FIG. 31A and FIG. 31G) 10 minutes after exposure to either 1 μM (FIG. 31B and FIG. 31H), 1.5 μM (FIG. 31C, FIG. 31I), 2 μM (FIG. 31D and FIG. 31J), 2.5 μM (FIG. 31E and FIG. 31K), or 5 μM (FIG. 31F and FIG. 31L) of HS-PEG₆-OCH₃.

FIG. 32 graphically illustrates data showing evolution over time of the dissociation efficiency of different concentrations of HS-PEG₆-OCH₃ added to Arg-Arg-AuNPs (40 nm).

FIG. 33 graphically illustrates data showing dissociation efficiency of HS-PEGx-OCH₃ of different molecular weights added to Arg-Arg-AuNPs (40 nm).

FIG. 34 graphically illustrates data showing the dissociation efficiency (%) of HS-PEG₆-OCH₃ added to Arg-Arg-AuNPs (40 nm) in different complex matrices.

FIG. 35 schematically illustrates an exemplary scheme for the assembly of citrate-capped AuNPs (AuNPs-citrate) with a di-arginine peptide and subsequent dissociation with thiolate dPEG molecules (HS-PEGs).

FIG. 36A-G illustrates short cationic peptides for reversible aggregation:

FIG. 36A is a schematic illustration of RRK-based particle aggregation, the inset photograph shows color change from red to blue as a function of RRK peptide (2-10 μM);

FIG. 36B illustrates TEM images of citrate-coated AuNPs (left) and RRK-induced AuNP aggregates (right);

FIG. 36C graphically illustrates UV-vis spectrum of RRK-induced AuNP aggregates;

FIG. 36D graphically illustrates Raman shifts before and after adding RRK peptides into citrate-coated AuNPs;

FIG. 36E graphically illustrates ratiometric signal (λ₅₂₀/λ₇₀₀) of AuNPs after adding R (the amino acid reside arginine), RRK, and RRKRRK (SEQ ID NO: 13) peptides with different concentrations 0.5-32 μM, respectively;

FIG. 36F graphically illustrates SMD simulations for free energy investigation as a function of AuNP distance at 298K and 1 atm, where black, red, and blue lines indicate a citrate to RRK molar ratios of 1:0, 9:1, and 1:1, respectively, and the inset images indicate the simulation stages along a trajectory of the citrate-coated AuNPs with RRK (9:1) system; and

FIG. 36G graphically illustrates metadynamics (MTD) free energy investigation for a system with 1 RRK on Au(111) surface (right) and a system with 1 citrate on Au(111) surface (left),
as discussed in further detail in Example 2, below.

FIG. 37A-I illustrates peptide-enabled dissociation of AuNP aggregates:

FIG. 37A is a schematic illustration of peptide-based particle dissociations, the peptide is EEKKPPC (SEQ ID NO:18);

FIG. 37B is an illustration of a UV-vis spectrum that shows that the plasmonic resonance peak of AuNP aggregates blue-shifted upon addition of the A1 peptide (see Table of FIG. 63);

FIG. 37C graphically illustrates a hydrodynamic diameter, and the surface charge after adding the A1 peptide;

FIG. 37D illustrates time-dependent photographs show 150 μM of the A1 peptide requires to dissociate AuNP aggregates, x and y axis indicate time and the A1 concentration, respectively;

FIG. 37E illustrate darkfield images of AuNP aggregates (left) and the dissociated AuNPs (right), the scale bar indicates 10 μm, and Blue dots represent actual AuNPs dissociated by the A1 peptide;

FIG. 37F graphically illustrates time-dependent particle dissociation driven by the A1 peptide;

FIG. 37G graphically illustrates particle dissociation was quenched without Cys (A2), and acetylation (A3);

FIG. 37H graphically illustrates Dissociation capacity of the A1, A5, A6, A7 and A8 peptides (see Table of FIG. 63); and

FIG. 37I graphically illustrates the dissociated AuNPs by the A1 (red) showed higher colloidal stability than citrate-coated AuNPs (black), as discussed in further detail in Example 2, below.

FIG. 38A-H illustrate the impact of hydrophilicity and steric bulk on particle dissociation:

FIG. 38A illustrates different Pro-, Ala-, Gly-spacers have different nature of rigidity and hydrophilicity which can impact on the dissociation capacity, peptide illustrated is KKEE (SEQ ID NO: 19);

FIG. 38B illustrates Table 2, which describes peptide sequences that are designed to investigate the impact of spacers, including peptides: EEKC (SEQ ID NO:20), EEKKPPC (SEQ ID NO: 18), EEKKAAC (SEQ ID NO:22), EEKKGGC (SEQ ID NO:23), EEKKGGGGC (SEQ ID NO:24), EEKKGGGGGGC (SEQ ID NO:25);

FIG. 38C illustrates photographs of the dissociated AuNPs by the PP, AA, GG spacers and without spacer (−) as a negative control;

FIG. 38D illustrates time-dependent particle dissociations driven by the A1, A9, A10, and A11 peptides (see Table of FIG. 63), respectively;

FIG. 38E illustrates Gly spacer showed the higher dissociation capacity than the Pro- and Ala- spacers;

FIG. 38F illustrates aggregation parameter of the dissociated AuNPs driven by the A1, A9, A10, and A11 peptides;

FIG. 38G illustrates FTIR data of the dissociated AuNPs by the A1, A9, A10, and A11 peptides; and

FIG. 38H illustrates the impact of the spacer length on the particle dissociation. Increasing the length of the spacer (from two to four) improved dissociation capacity while the spacer with six Glu (i.e., A13) showed lower dissociation capacity than A12 peptide, as discussed in further detail in Example 2, below.

FIG. 39A-H illustrate M^{pro} detection using an exemplary dissociation strategy:

FIG. 39A illustrates a schematic showing that M^{pro} cleavage releases dissociation domains, changing the color from blue to red, the illustrated exemplary dissociation peptide (i.e., A18) comprises three parts: dissociation domain (CGGKKEE) (SEQ ID NO:26), cleavage site between residues Q and S on peptide AVLQSGF (SEQ ID NO:29), or (AVLQJSGF), with the full length peptide being CGGKKEEAVLQSGF (SEQ ID NO:27), and dissociation shielding site (R), and the inset images are before and after particle dissociation obtained by darkfield microscopy, the blue dots indicate actual AuNP aggregates (left) and the dissociated AuNPs (right);

FIG. 39B illustrates color changes with (+) and without (−) M^{pro} in PB buffer, where the released A18 fragment (CGGKKEEAVLQ) (SEQ ID NO:27) dissociated AuNP aggregates, changing the color from blue to red;

FIG. 39C illustrates MALDI-TOF MS data before and after M^{pro} cleavage, confirming the mass peaks of the A18 parent and its fragment;

FIG. 39D illustrates time-dependent particle dissociation by the A18 fragments;

FIG. 39E illustrates UV-vis spectrum before and after particle dissociation by the A18 fragments with different concentrations (8-80 μM);

FIG. 39F illustrates changes in the size and surface charge after the dissociation induced by M^{pro} cleavage, where Table 3 (FIG. 39G) describes peptide sequences that are designed to confirm the best location and order of the dissociation domain for M^{pro} detection;

FIG. 39G illustrates Table 3; and,

FIG. 39H illustrates particle dissociations driven by the A14, A15, A16, and A17 peptides,
as discussed in further detail in Example 2, below.

FIG. 40A-K illustrate matrix-insensitive M^{pro} detection:

FIG. 40A illustrates a schematic illustration of a matrix insensitive M^{pro} detection where the released A18 fragment by M^{pro} cleavage was used for colorimetric biosensing in saliva or EBC;

FIG. 40B graphically illustrates time-dependent M^{pro} detection from 0 to 47 nM in saliva, the inset photograph shows that our dissociation strategy can provide a clear readout of positive M^{pro} sample above 11 nM in saliva;

FIG. 40C graphically illustrates detection limits of M^{pro} in saliva, EBC, and PB buffer, respectively, and illustrates a Table 4 that describes peptide sequences (peptide A18 is CGGKKEEAVLQSGFR ((SEQ ID NO:30)), and peptide A19 is CGGKKEEAVLQSGF ((SEQ ID NO:27))) that are designed to verify the role of dissociation screening domain;

FIG. 40D graphically illustrates one Arg at the C-terminus can prevent false positives;

FIG. 40E graphically illustrates A18 fragment from C terminus (i.e., SGFR (SEQ ID NO:31)) had negligible impact on the dissociation process;

FIG. 40F graphically illustrates an exemplary specificity test using multiple different biological essays (for example, inactivated M^{pro} (inact M^{pro}), hemoglobin (Hg), Thrombin (Thr), BSA, Saliva, Amalyase (Amal));

FIG. 40G graphically illustrates an exemplary GC376 inhibitor assay test in saliva, EBC, and PB buffer, respectively;

FIG. 40H graphically illustrates data showing that the released A18 fragments by M^{pro} cleavage can dissociate other types of plasmonic assemblies such as AgNP aggregate;

FIG. 40I graphically illustrates data showing that after particle dissociation, the A18-capping AuNPs maintained high colloidal stability in different biological (for example, urine, saliva, plasma, DMEM) and extreme condition (for example, 2M NaCl);

FIG. 40J graphically illustrates the plasmonic resonance peaks of the AuNP aggregates blue-shifted after particle dissociation in 100% of 1. human urine, 2. plasma, 3. seawater, and 4, saliva, the inset photographs show before (left) and after (right) adding dissociation peptides;

FIG. 40K graphically illustrates ratiometric signal (λ₅₂₀/λ₇₀₀) of the dissociated AuNPs in diverse matrixes, indicating that our dissociation strategy is less affected by sample matrix,
as discussed in further detail in Example 2, below.

FIG. 41 schematically illustrates molecular modeling of the energy level of nanoparticle aggregation as a function of distance including an optimal spacing distance; the illustration shows how the protease can separate the peptide and cause the particles to resuspend thus leading to a color change.

FIG. 42 illustrates RRK-induced AuNP aggregates, and RRK peptides with 2 to 10 μM were used to aggregate AuNPs; and dynamic light scattering (DLS) data showed that the size of citrate-coated AuNPs were aggregated by the RRK peptides,
as discussed in further detail in Example 2, below.

FIG. 43A-B illustrate interactions between RRK and citrate molecules:

FIG. 43A schematically illustrates molecular dynamics (MD) snapshots of a citrate-coated Au nanoparticle system after adding RRK molecules. Na ions, Cl ions, and water molecules are not shown for clarification; and

FIG. 43B schematically illustrates simulated charge density indicated that molecular interactions between RRK peptides and citrate readily occurred at a tail portion rather than the middle portion due to atomic charge effects,
as discussed in further detail in Example 2, below.

FIG. 44A-B illustrate free energy investigation as a function of AuNP distance:

FIG. 44A graphically illustrates SMD simulation for a system with bare two AuNPs in water, the x axis indicates the center of mass between two bare AuNPs; and

FIG. 44B schematically illustrates the initial and final snapshots of two bare AuNPs are shown,
as discussed in further detail in Example 2, below.

FIG. 45A-C illustrate TEM images of AuNP aggregates and particle dissociations:

FIG. 45A illustrates an image of AuNP aggregates induced by RRK peptides;

FIG. 45B illustrates an image taken after adding the dissociation peptides (A1, Ace-EEKKPPC-Am) (SEQ ID NO:18) the aggregated AuNPs were re-dispersed; and

FIG. 45C illustrates a high-magnified TEM image in the red-dotted box in panel b, 10 μM of RRK peptide was used to induce particle aggregation,
as discussed in further detail in Example 2, below.

FIG. 46A-H illustrate ESI-MS data of eight peptide sequences (A1-A8) to define the role of amino acid in dissociation peptide ESI-MS data of:

FIG. 46A: A1 (Ace-EEKKPPC-Am) (SEQ ID NO:18),

FIG. 46B: A2 (Ace-EEKKPPG-Am) (SEQ ID NO:32),

FIG. 46C: A3 (NH2-EEKKPPG-Am) (SEQ ID NO:32),

FIG. 46D: A4 (Ace-KEEKKPPC-Am) (SEQ ID NO:21),

FIG. 46E: A5 (Ace-KEEPPKKC-Am) (SEQ ID NO:33),

FIG. 46F: A6 (Ace-KKEEPPC-Am) (SEQ ID NO:34),

FIG. 46G: A7 (Ace-EEPPC-Am) (SEQ ID NO:35), and

FIG. 46H: A8 (Ace-KKPPC-Am) (SEQ ID NO:36),
as discussed in further detail in Example 2, below.

FIG. 47A-C illustrate the role of cysteine and acetylation in the particle dissociation:

FIG. 47A illustrates data showing that an A1 peptide with the concentration of 150 and 300 μM dissociated AuNP aggregates;

FIG. 47B illustrates data showing dissociation action of an A2 peptide (i.e., without cysteine); and

FIG. 47C illustrates data showing dissociation action of an A3 peptide (free amine at the N-terminus), which failed to dissociate AuNP aggregates,
as discussed in further detail in Example 2, below.

FIG. 48A-B illustrate free amine at N terminus prevents particle dissociation: to further confirm that free amine at N terminus can inversely impact on particle dissociation, we synthesized the A4 peptide which contains Lys at the C terminus. Not surprisingly, the A3 peptide EEKKPPC (SEQ ID NO:18) (FIG. 48A) and the A4 peptide KEEKKPPC (SEQ ID NO:21) (FIG. 48B) peptide failed to dissociate AuNP aggregates, meaning that positive charged peptides are incapable of dissociating AuNP aggregates, as discussed in further detail in Example 2, below.

FIG. 49A-B illustrate ESI-MS data A9-A13 peptides:

FIG. 49A illustrates for the A9 peptide (Ace-EEKKC-Am) (SEQ ID NO:37),

FIG. 49B illustrates for the A10 peptide (Ace-EEKKAAG-Am) (SEQ ID NO:38),

FIG. 49C illustrates for the A11 peptide (Ace-EEKKGGC-Am) (SEQ ID NO:23),

FIG. 49D illustrates for the A12 peptide (Ace-EEKKGGGGC-Am) (SEQ ID NO:24), and

FIG. 49E illustrates for the A13 peptide (Ace-EEKKGGGGGGC-Am) (SEQ ID NO:25),
as discussed in further detail in Example 2, below.

FIG. 50 illustrates photographs of the dissociated AuNPs by A1, A9, A10, A11 peptides the AuNP aggregates were dissociated using A1 (Ace-EEKKPPC-Am) (SEQ ID NO:18), A9 (Ace-EEKKC-Am) (SEQ ID NO:37), A10 (Ace-EEKKAAC-Am) (SEQ ID NO:22), A11 (Ace-EEKKGGC-Am) (SEQ ID NO:23). The color changed from blue to red due to the particle dissociation, as discussed in further detail in Example 2, below.

FIG. 51A-B illustrate the impact of spacer lengths on the particle dissociation:

FIG. 51A illustrates time-dependent particle dissociations using A11 (Ace-EEKKGGC-Am) (SEQ ID NO:23), A12 (Ace-EEKKGGGGC-Am) (SEQ ID NO:24), and A13 (Ace-EEKKGGGGGGC-Am) (SEQ ID NO:25) peptides; and

FIG. 51B illustrates data showing that particle dissociation was attenuated likely due to the increased steric hinderance by length, used peptides GG, GGGG (SEQ ID NO:39) and GGGGGGG (SEQ ID NO:40),
as discussed in further detail in Example 2, below.

FIG. 52A-D illustrate ESI-MS data of A14-A17 peptides: ESI-MS data of: FIG. 52A for the A14 peptide (Ace-SGFEEKKGGC-Am) (SEQ ID NO:40), FIG. 52B for the A15 peptide (Ace-CGGKKEEAVLQ-Am) (SEQ ID NO:27), FIG. 52C for the A16 peptide (Ace-CGGKEAVLQ-Am) (SEQ ID NO:41), and FIG. 52D for the A17 peptide (Ace-EEKKGGCAVLQ-Am) (SEQ ID NO:42), as discussed in further detail in Example 2, below.

FIG. 53A-D illustrate the impact of the fragment sequence, length, and the location of cysteine on the particle dissociation: FIG. 53A for the SGFEEKKGGC (SEQ ID NO:40) peptide, FIG. 53B for the AVLQEEKKGGC (SEQ ID NO:43) peptide, FIG. 53C for the AVLQEKGGC (SEQ ID NO:44) peptide, FIG. 53D for the AVLQCGGKKEE 45) peptide, as discussed in further detail in Example 2, below.

FIG. 54A-B illustrate dissociation strategy applied in saliva and EBC: Mpro cleaved the dissociation peptides (i.e., A18, Ace-CGGKKEEAVLQSGFR-Am) (SEQ ID NO:30) in saliva (FIG. 54A), EBC (FIG. 54B), and the fragments dissociated AuNPs, changing color to reddish, the UV-vis spectrum showed the dissociated AuNPs with the different Mpro concentrations, as discussed in further detail in Example 2, below.

FIG. 55A-B illustrate ESI-MS data of A18-19 peptides: ESI-MS data of: FIG. 55A for the A18 peptide (Ace-CGGKKEEAVLQSGFR-Am) (SEQ ID NO:30); FIG. 55A for the A19 peptide (Ace-CGGKKEEAVLQSGF-Am) (SEQ ID NO:27), as discussed in further detail in Example 2, below.

FIG. 56A-B illustrate Arg residue at the C terminus for charge-screening: to prevent false positive, the dissociation peptide was charge-screened using Arg at the C terminus. AuNP aggregates were incubated with the different concentrations (from 2 to 100 μM) of A18 (with R) and A19 (without R) peptides: FIG. 56A illustrates time-dependent particle dissociations and their UV-vis spectrum (FIG. 56B); the results showed that the A19 peptide dissociated AuNP aggregates without Mpro (i.e., false positive) while A18 peptide which included Arg residue prevents the false positive.

FIG. 57 illustrates the impact of the SGFR fragment on particle dissociation: after Mpro cleavage, the A18 fragments (i.e., CGGKKEEAVLQ (SEQ ID NO:27)) dissociated AuNP aggregates. SGFR showed negligible impact on particle dissociation. UV-vis spectrum showed that the dissociated AuNPs by the A18 fragments maintained high colloidal stability in the presence of SGFR fragments from 2 to 200 μM.

FIG. 58 illustrates UV-vis spectrum of the specificity test: the inactive Mpro (incubated 60° C. for 3 h), BSA, hemoglobin, thrombin, α-amylase, saliva and Mpro were incubated with the dissociation peptide (i.e., A18) for 1 h at 37° C.; after then, AuNP aggregates were used for the specificity test. The UV-vis spectrum showed that only Mpro dissociated AuNP aggregates due to the release of the dissociation peptides while other enzymes and proteins did not induce the particle dissociations.

FIG. 59 illustrates UV-vis spectrum of the inhibition test: the GC376 inhibitor was incubated with Mpro for the inhibition test. The 0, 0.2, 0.5, 1, 2, 5, and 10 represent the ratio between inhibitor to Mpro (200 nM). The results showed that GC376 inhibited Mpro proteolytic activity, preventing particle dissociation.

FIG. 60A-B illustrate the GC376 inhibitor with AuNP aggregates: the GC376 inhibitor (from 0.1 to 20 mM) was incubated with AuNP aggregates. UV-vis spectrum (FIG. 60A) and time-dependent particle dissociation (FIG. 60A) showed that the inhibitor itself had no impact on the particle dissociation.

FIG. 61A-B illustrate dissociation strategy using 20 nm AgNPs: FIG. 61A illustrates UV-vis spectrum and FIG. 61B illustrates DLS data of the dissociated AgNP aggregates using the dissociation peptides; the A18 fragments released by Mpro cleavage (20-200 μM) were used for the dissociation test; these results showed that our dissociation strategy could be applied using AgNPs which provide a broader absorption shift (from blue to yellow).

FIG. 62A-C illustrate particle dissociation in 100% human urine, plasma, seawater, and saliva: UV-vis spectrum of AuNP aggregates (FIG. 62A) and the dissociated AuNPs (FIG. 62B) after adding dissociation peptide (i.e, A11) in 100% human urine, plasma, seawater, and saliva; FIG. 62C illustrate photographs that show that our dissociation peptide can re-disperse AuNP aggregates, offering color changes from blue to red in diverse matrixes.

FIG. 63 illustrates Table 2, or exemplary properties of exemplary peptides A1 (comprising EEKKPPC (SEQ ID NO: 18)), A2 (comprising EEKKPPG (SEQ ID NO:32)), A3 (comprising EEKKPPC (SEQ ID NO: 18)), A4 (comprising KEEKKPPC (SEQ ID NO:21)), A5 (comprising EEPPKKC (SEQ ID NO:45)), A6 (comprising KKEEPPC (SEQ ID NO:34)), A7 (comprising EEPPC (SEQ ID NO:35), and, A8 (comprising KKPPC (SEQ ID NO:36).

FIG. 64 illustrates Table 1, showing the impact of polyethylene glycol (PEG) structure on dissociation capacity, i.e., ligands differing either by their size, anchoring group, core, or charge, as discussed in further detail in Example 1, below.

Like reference symbols in the various drawings indicate like elements.

### DETAILED DESCRIPTION

In alternative embodiments, provided are compositions, including products of manufacture and kits, and methods, for detecting proteases including detecting proteases in a biological sample.

In alternative embodiments, provided are a compound X capable of making or forming into reversibly aggregated nanoparticles (NPs), or NP assemblies, that comprise:
a nanoparticle (NP) aggregated or stabilized with (Arginine)ₓ (or Argₓ or Rₓ), or an Argₓ citrate-stabilized nanoparticle, or a plurality of Argₓ citrate-stabilized nanoparticles (NPs), or Argₓ-NPs, wherein x is an integer 2, 3, 4, 5 or 6, and optionally the Rₓ comprises AA, AAA, AAAA, AAAAA or AAAAAA, or optionally the NP comprises a di-arginine (Arg) citrate-stabilized nanoparticle, or a plurality of di-arginine (Arg) citrate-stabilized nanoparticles (NPs), or Arg-Arg-NPs,
a nanoparticle (NP) aggregated or stabilized with Lysineₓ-Rₓ (or -Lysₓ-Rₓ, or Kₓ-Rₓ, or an Kₓ-Rₓ citrate-stabilized nanoparticle, or a plurality of Kₓ-Rₓ citrate-stabilized nanoparticles (NPs), or Kₓ-Rₓ-NPs, wherein x is an integer 1, 2, 3, 4, 5 or 6, and optionally the Kₓ-Rₓ comprises KR, RK, RRK, KRR, RKR, RRKR, RKRR, KRRR, RRRK, KKRR, KKKR, RKKK, KRRK or RKKR,
and compound X aggregates (or is capable of aggregating) when in a liquid solution, and optionally the liquid solution comprises (a) a biological fluid, optionally a biological fluid from an in vivo source, optionally an undiluted and/or untreated biological fluid from an in vivo source, and optionally the biological fluid from an in vivo source comprises blood, plasma, saliva, urine, bile, a lacrimal duct solution (a tear), or cerebrospinal fluid (CSF); (b) a cell lysate; or (c) water, distilled water, saline or sea water,
wherein optionally compound X, or the citrate-stabilized compound X, or the citrate-stabilized nanoparticle (NP) comprises or is conjugated to a metal to generate a metal-nanoparticle or metal-compound X, and optionally the metal of the metal nanoparticle or metal-compound X comprises silver (Ag).

In alternative embodiments, the nanoparticles comprise any fragment or piece of a metal, for example, having one dimension that is 100 nm diameter or less, or a diameter of between about 1 nm and 300 nm, or a diameter of between about 5 nm and 200 nm, or a diameter of between about 10 nm and 150 nm, or a diameter of between about 20 nm and 100 nm, or the nanoparticles have a dimension or a diameter of about 1, 2, 3, 4, 5, 6, 7, 8, 9 or 10 nm. In alternative embodiments, the nanoparticles are in the same oxidation state as larger pieces of metal except the surface atoms may be different that the core atoms. In alternative embodiments, the nanoparticles are spherical or rod-shaped, or may have a more exotic shape, such as for example, being star-shaped, sea-urchin-shaped, and the like. In alternative embodiments nanoparticles as used here are made of (or comprise) silver (Ag), gold (Au), platinum (Pt), palladium (Pd), copper (Cu), iron (Fe), zinc (Zn) and/or any other precious metals. In alternative embodiments, the nanoparticles are formed as colloids (for example, with a polymer such as polyvinyl pyrrolidone), or conjugated with an organic ligand (for example, with a quaternary amine), or as a micelle (for example, with a lipid). In alternative embodiments, the nanoparticles comprise a precious metal coated on a nonprecious metal nanoparticle, for example, Au, Ag or Pt on cobalt (Co). In alternative embodiments, the nanoparticles comprise biodegradable nanospheres, for example, comprise albumin nanospheres, polypropylene dextran nanospheres, gelatine nanospheres, modified starch nanospheres, and polylactic acid nanospheres, poly-lactic acid (PLA), poly-D-L-glycolide (PLG), poly-D-1-lactide-co-glycolide (PLGA), dipalmitoyl-phosphatidylcholine (DPPC) and/or poly-cyanoacrylate (PCA).

The assemblies can be dried without any loss of particles. The assembly of NPs-citrate changes their optical properties and the color of the suspension turns from red (not aggregated, or not assembled) to blue (aggregated, or assembled).

In alternative embodiments, provided herein are chimeric compositions Z-PEGₓ-Y-peptide, comprising dipeptides comprising two arginines (Arg-Arg) that are capable of inducing the assembly of citrate-capped gold nanoparticles (NPs) (NPs-citrate); and the resulting Arg-Arg-NPs (blue colored) are stable over time as the peptide protects the particles from degradation.

In alternative embodiments, the assemblies, or aggregated nanoparticles, are dissociated with Z-PEGₓ, or thiolated, polyethylene glycol (HS-PEGs) molecules, or thiol-conjugated peptides, which leads to the recovery of the initial optical properties of the NPs, for example, AgNPs or AuNPs, i.e., the red color of the suspension.

We have observed that such dissociation of NP assemblies, or aggregated nanoparticles, is not sensitive to the composition of the medium, and it can thus be performed in biological fluids such as pure plasma, saliva, urine, bile, cell lysates or even sea water, which contrasts with previously used nanoparticles-based sensing platforms which all lack the capacity to operate in biological fluids due to background signal caused by endogenous molecules.

We also have demonstrated how the NP assembly dissociation, or dissociation of aggregated nanoparticles (for example, dissociation of Arg-Arg-NPs) with thiol-conjugated peptides, or Z-PEGₓ can be exploited for biomarker sensing.

We have shown that it is possible to conjugate, or link, a peptide substrate to Z-PEGₓ molecules, making the resulting conjugate (Z-PEGₓ-Y-peptide) specific to a target protease. The presence of the protease is thus detected via the proteolytic cleavage that releases Z-PEGₓ fragments (from Z-PEGₓ-peptide), inducing the dissociation of the compound X assemblies (or AuNPs-citrate assemblies) and turning the solution from blue to red. In the absence of proteases, the Z-PEG-peptide cannot dissociate the NPs-citrate assemblies. Data presented in Example 1, demonstrates the efficacy of compositions and methods as provided herein to detect a model protease, trypsin, and a viral protease, i.e., the main protease of the virus SARS-CoV-2 (Mpro), in saliva.

Data presented in Example 1, the detection of protease biomarker is performed by the conjugation of a peptide substrate to a HS-PEGs molecule. The resulting HS-PEG-peptide conjugate is either too bulky or too charged to dissociate the AuNPs assemblies. However, in the presence of the target protease, the HS-PEG-peptide conjugate is cleaved by the target protease, resulting in release of a HS-PEGs fragment that can dissociate the AuNP assemblies, resulting in an unambiguous color change. The dissociation is complete after 30 minutes and the color change can be observed after only 5 minutes. This can be performed in any biological fluid and can be adapted to any protease as far as it is possible to find a peptide substrate.

Data presented in Example 1 demonstrates the reversible aggregation of gold nanoparticle (AuNPs) assemblies via a di-arginine peptide additive and thiolated PEGs (HS-PEGs). The AuNPs were first aggregated by attractive forces between the citrate-capped surface and the arginine side chains. We found that HS-PEG thiol group has higher affinity for the AuNPs surface, thus leading to redispersion and colloidal stability. In turn, there was a robust and obvious color change due to on/off plasmonic coupling. The assemblies' dissociation was directly related to the HS-PEG structural properties such as their size or charge. As an example, HS-PEGs with a molecular weight below 1 kDa could dissociate 100% of the assemblies and restore the exact optical properties of the initial AuNPs suspension (prior to the assembly). Surprisingly, the dissociation capacity of HS-PEGs was not affected by the composition of the operating medium and could be performed in complex matrices such as plasma, saliva, bile, urine, cell lysates or even sea water. The high affinity of thiols for the gold surface encompasses by far the one of endogenous molecules and is thus favorized. Moreover, starting with AuNPs already aggregated ensured the absence of background signal as the dissociation of the assemblies was far from spontaneous. Remarkably, it was possible to dry the AuNPs assemblies and to solubilize them back with HS-PEGs, improving the colorimetric signal generation. We used this system for protease sensing in biological fluid. Trypsin was chosen as model enzyme and highly positively charged peptides were conjugated to HS-PEG molecules as cleavage substrate. The increase of positive charge of the HS-PEG-peptide conjugate quenched the dissociation capacity of the HS-PEG molecules which could only be restored by the proteolytic cleavage. Picomolar limit of detection was obtained as well as the detection in saliva or urine.

Data presented in Example 1 demonstrates a novel process of reversible aggregation of AuNPs-citrate for alternative sensing strategies. Reversible aggregation of nanoparticles is challenging because, according to the Derjaguin-Landau-Verwey-Overbeek (DLVO) theory,²¹ the particles can be trapped in a deep energetic minima during the aggregation, thus transforming the aggregates into larger insoluble materials that can be only slightly dissociated by aggressive sonication; their optical properties cannot be recovered.²² While many in the community have shown reversible assembly of a small number of nanoparticles, this usually requires sophisticated coating of the AuNPs-citrate surface with responsive polymers,^{23,24} DNA strands^{25,26} or other organic ligands.^{27,28} However, we are unaware of work describing the assembly of large amount of AuNPs-citrate into macroscopic aggregates that can easily be dissociated without the need for prior surface modifications.

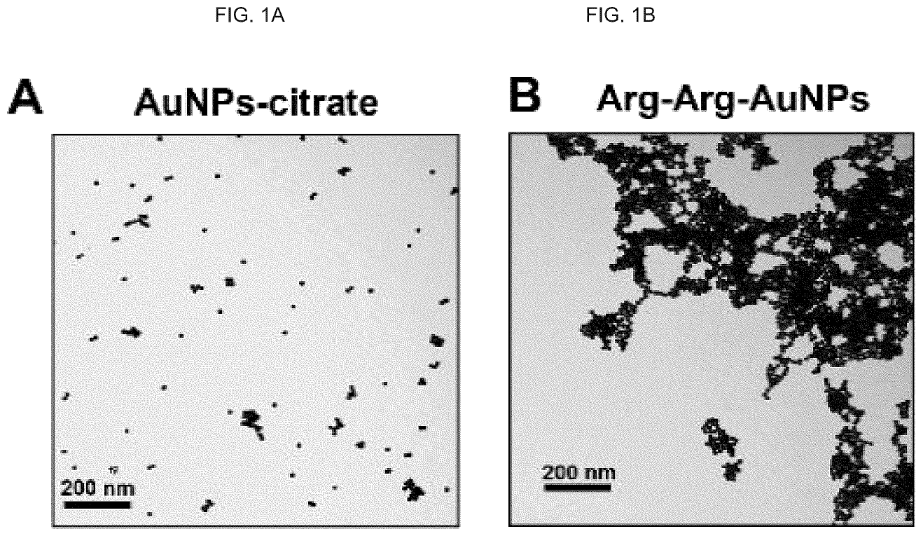

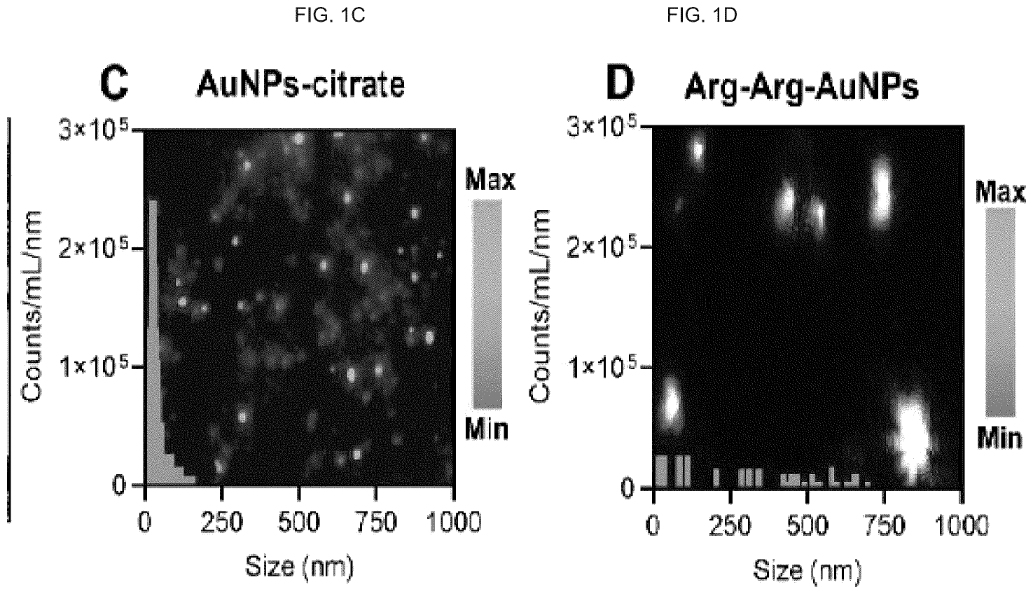

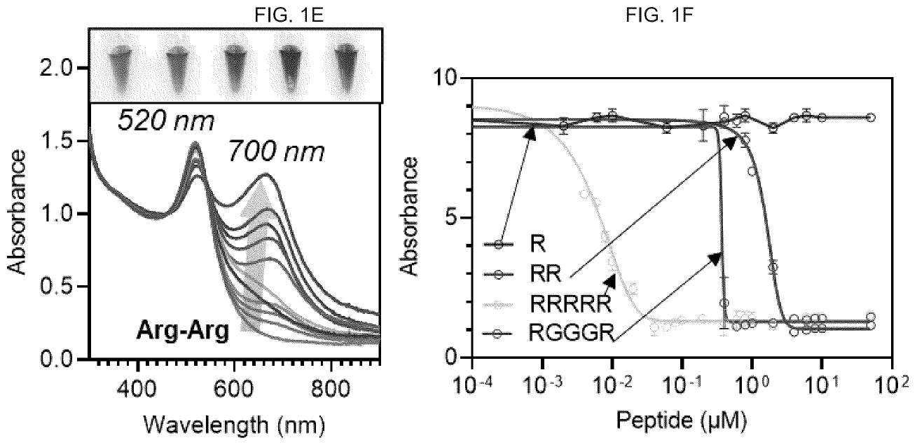

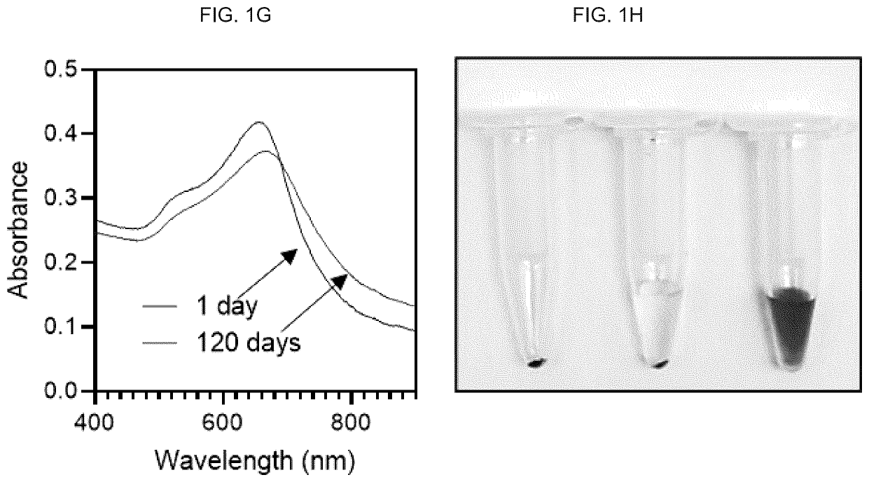

Data presented in Example 1 demonstrates use of a di-arginine additive that causes the aggregation of AuNPs and thiolated poly(ethylene glycol) (HS-PEGs) for the dissociation of the assemblies (Scheme 1, illustrated in FIG. 1). The system works through differences in affinity of the surface ligands: the introduction of HS-PEGs leads to redispersion of the AuNPs due to the higher affinity of the thiol for the gold surface than the arginine or citrate. In turn, there is a plasmonic color change. Starting with assembled AuNPs allows for insight on the colorimetric signal. Surprisingly, the redispersion can be performed over a wide variety of solvents including human plasma, serum, saliva, and even bile. After characterizing the ligands and HS-PEGs best suited for this reaction, we deployed it for colorimetric sensing of proteases via a cleave PEG-peptide conjugate. Although the grafting of HS-PEGs molecules on dispersed AuNPs has been widely reported in the literature, to the best of our knowledge, the dissociation of AuNPs assemblies with HS-PEGs has never been studied before, especially for the detection of proteases.

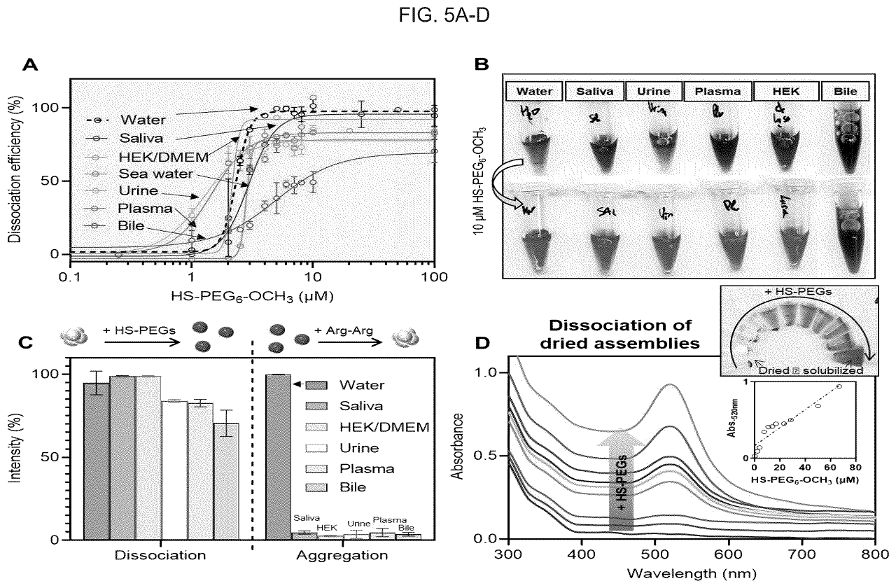

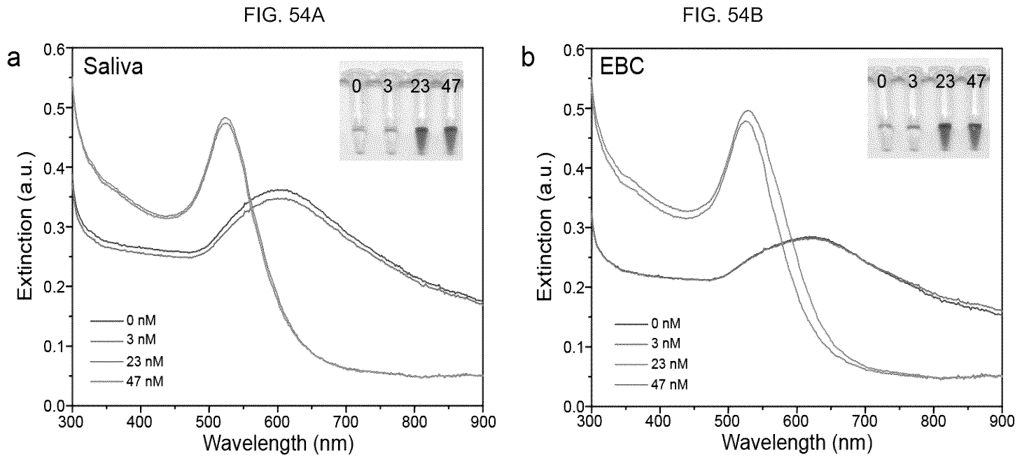

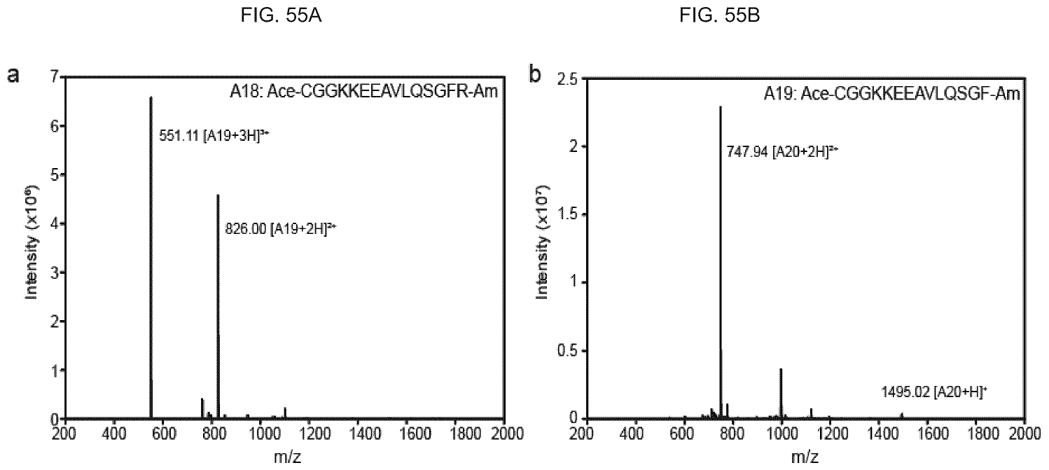

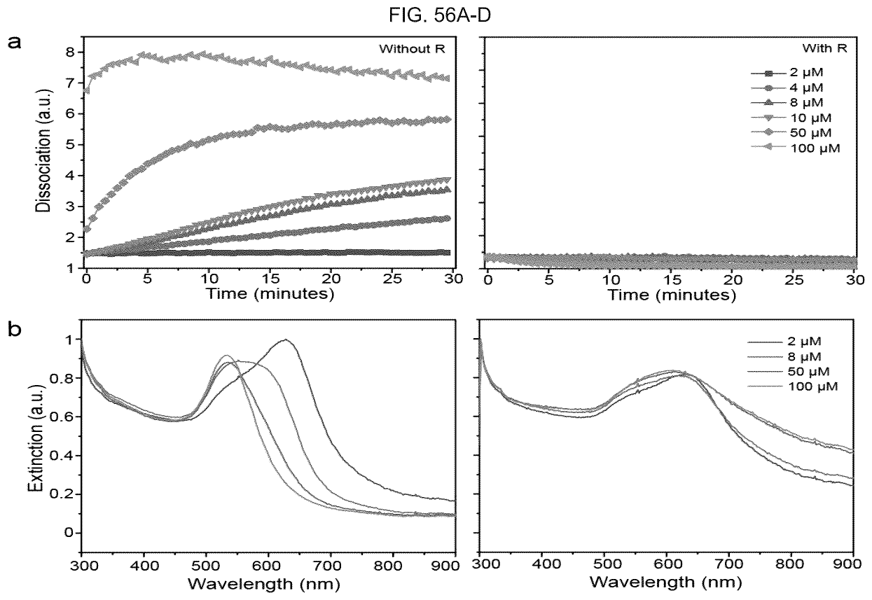

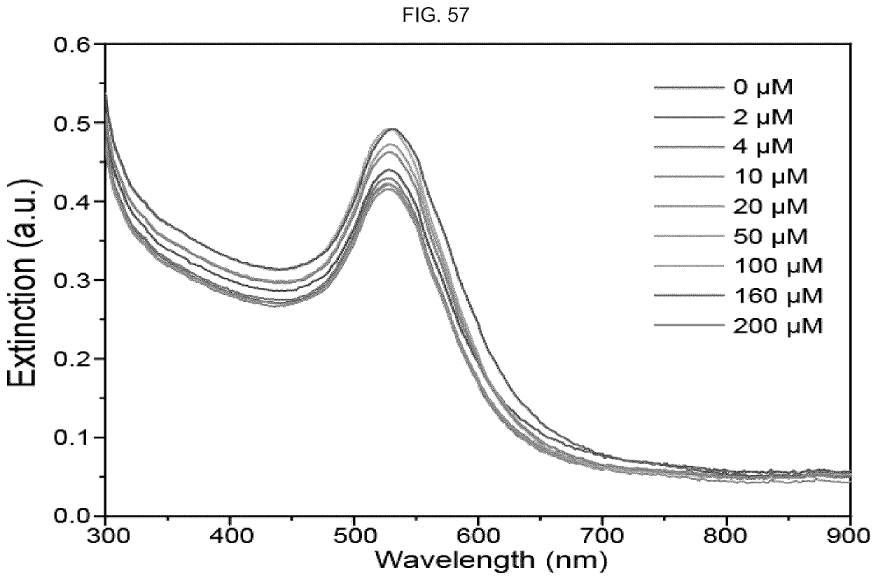

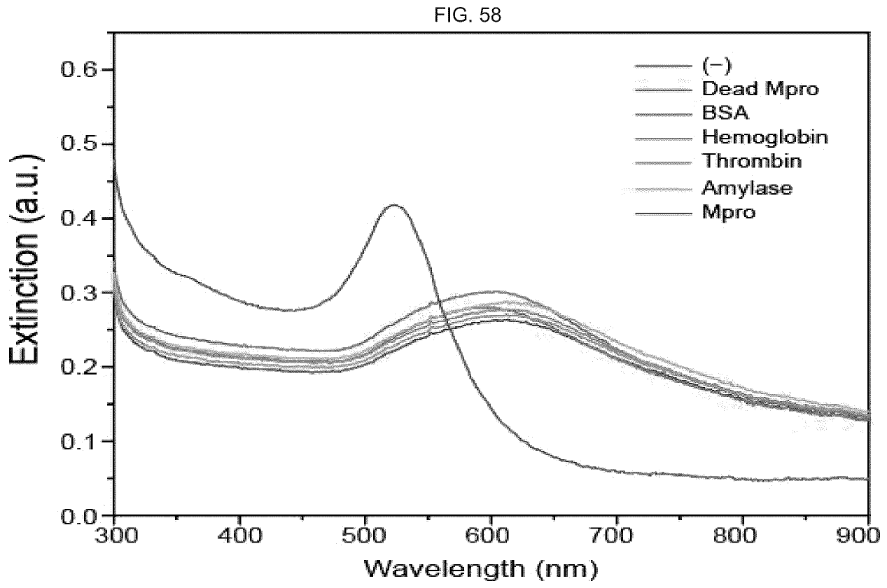

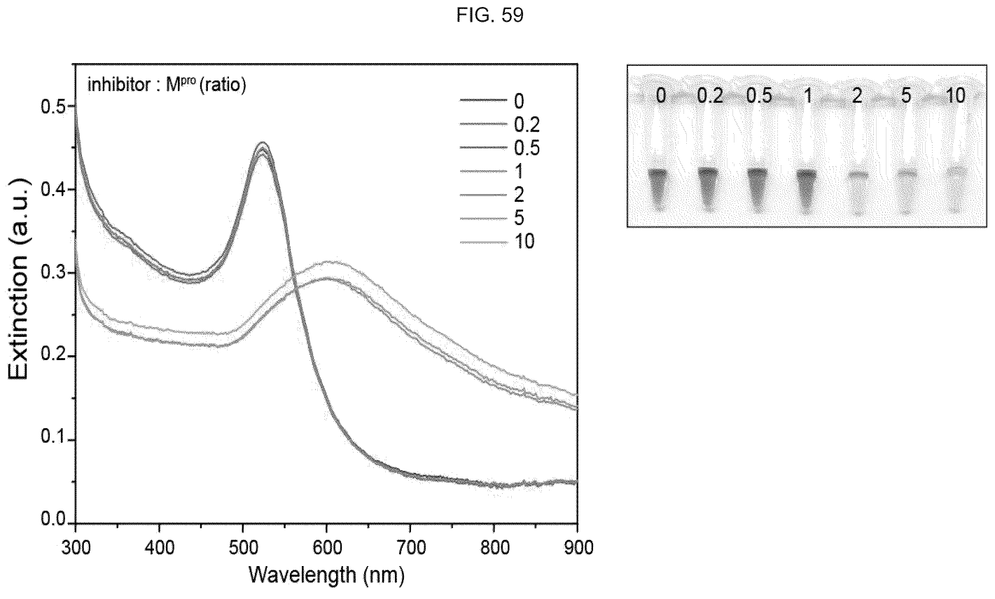

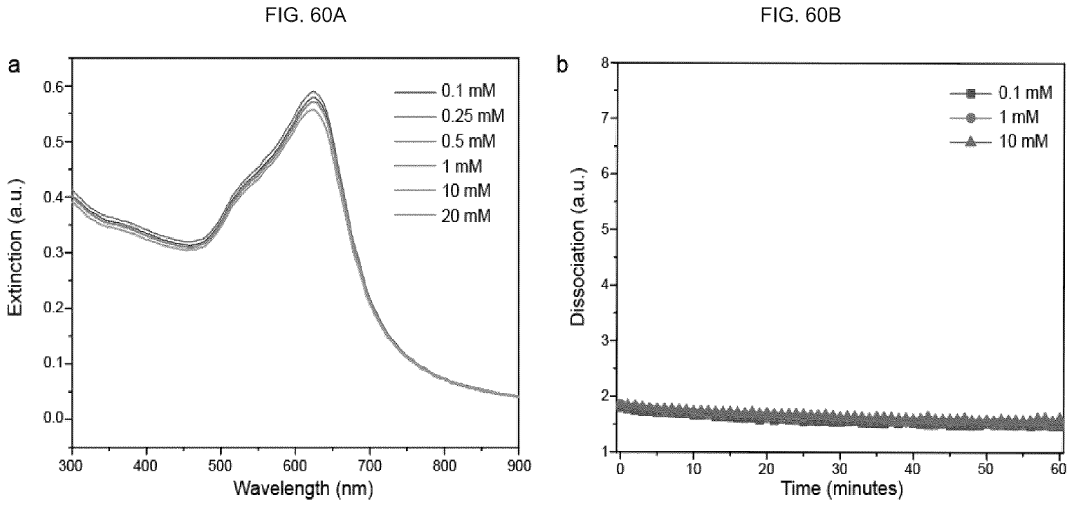

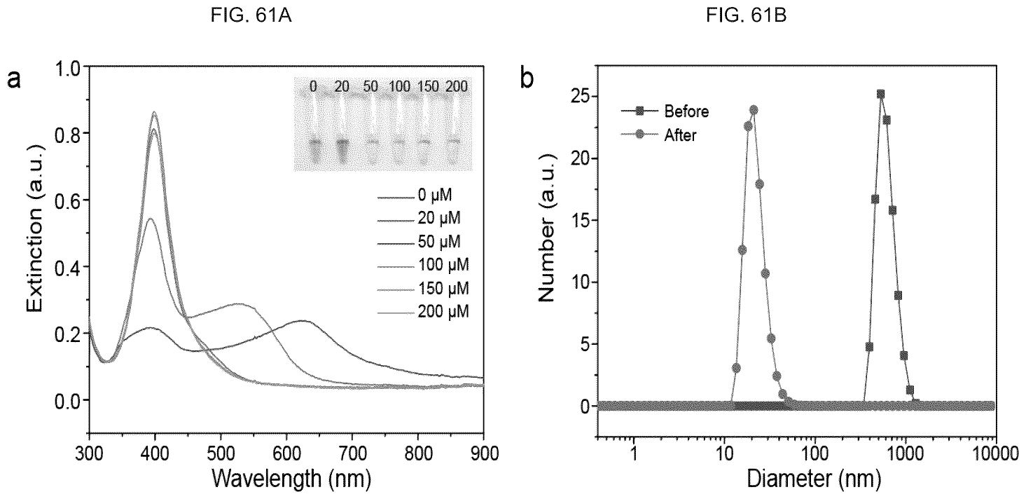

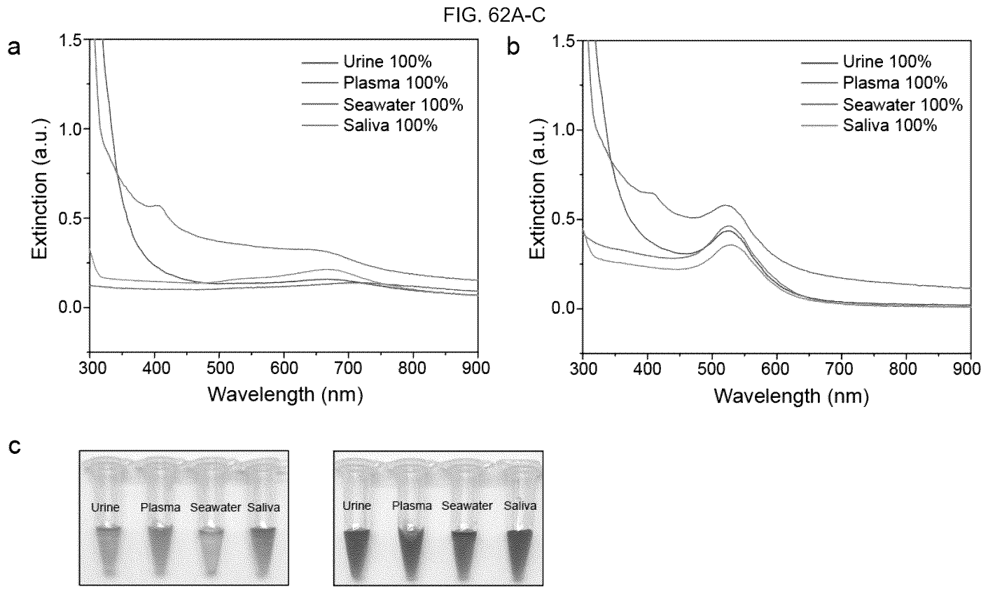

In alternative embodiments, one advantage of the dissociation approach as provided herein is that the assemblies did not just remain aggregated, they also precipitated, thus increasing the color change between the dissociated and aggregated systems. As an example, FIG. 54A-B shows that Arg-Arg-AuNPs suspended in saliva after 2 hour tend to precipitate, thus leading to a loss of blue color in the suspension while the dissociated particles remain bright red. Interestingly, even if precipitated, the assemblies can still be dissociated with 10 μM of HS-PEG6-OCH₃. Remarkably, the HS-PEGs could even solubilize dried films of Arg-Arg-AuNPs (FIG. 5D). Briefly, 50 μL of aqueous solution containing different concentrations of HS-PEGs, ranging from 0 to 70 μM, were added to the dried particles and 20 minutes later, the UV-Vis spectrum of the solution was recorded. In the absence of HS-PEGs, no LSPR band of the particles was observed as no particles detached spontaneously and the solution remained clear. However, in the presence of HS-PEGs, the plasmonic band of the particles was observed proportionally to the HS-PEGs concentration without the need of sonication. The HS-PEGs led to the spontaneous and progressive detachment of the AuNPs from the surface and thus a color change (FIG. 5D, inset). The solubilization of the AuNPs could be thus quantified using only the absorbance at 520 nm in the solution as the LSPR band intensity was linearly proportional to the HS-PEGs concentration. This approach did not work on AuNPs-citrate as they degraded during the drying process, the di-peptide is a crucial additive. Finally, the solubilization of dried Arg-Arg-AuNPs was possible even in biofluids such as saliva, plasma, urine or cell lysates (FIG. 55A-B). Thus, exemplary dissociation strategies as provided herein provide advantages versus traditional aggregation-based assays: they are insensitive to the composition of the operating medium; and, the gap of colorimetric signals between dissociated and non-dissociated samples is unambiguous and increases over time, thus enhancing the naked eyes identification.

In Example 1, the dissociation of AuNPs assemblies with HS-PEGs molecules was studied and exploited to build matrix-insensitive sensors. Robust assemblies of AuNPs-citrate were formed using a di-arginine additive (Arg-Arg). The efficient electrostatic interactions between the citrate and the arginine led to compact assemblies of the particles, thus provoking a strong modification of their optical properties. However, the presence of peptides protected the AuNPs from degradation. Surprisingly, the addition of HS-PEGs could dissociate the assemblies with a total recovery of the initial optical properties. The mechanism was fully characterized by TEM, MANTA, UV-Vis, and FTIR spectroscopies. The HS-PEGs can progressively graft onto the AuNPs surface and remove the citrate/arginine layers. As the hydrophilic PEG layer surrounds the AuNPs, the particles progressively detach from the bulky assemblies and become water-dispersible. Importantly, only a minimum amount of HS-PEGs is needed to cover all the gold surfaces (approximately 4 HS-PEG₆-OCH₃/nm²). We have thus shown that the dissociation capacity of HS-PEGs is modulated by their size and charge. HS-PEG-OCH₃ with a molecular weight of 1000 Da or less could dissociate 80% or more. The dissociation of the assemblies was matrix-insensitive and produced an unambiguous color change in plasma, saliva, urine, bile, cell lysates, or even sea water. Moreover, we found that the generation of the colorimetric signal could be improved by using dried film of AuNPs assemblies. The presence of HS-PEGs leads to the detachment of AuNPs from the surface as it solubilizes them. The color of the suspension becomes then red and its intensity is proportional to the amount of HS-PEGs. In the absence of this later, the color of the solution is clear. This strategy allows a unambiguous distinction with the naked eyes between samples that have or not HS-PEGs. We thus designed a sensing strategy based on HS-PEG-peptide probes and AuNPs assemblies as signal read out for protease sensing in complex media. Trypsin was chosen as model protease and peptide containing repetition of the motif RRK were conjugated to HS-PEGs. The optimized conjugate, HS-PEG-RRKRRK (SEQ ID NO:13), allowed the visual detection of trypsin with a picomolar limit of detection. Detection could be performed simply in pooled urine or saliva spiked with trypsin. This is the first time that HS-PEGs molecules have been used to dissociate AuNPs assemblies or to solubilize dried AuNPs and combined with peptides for protease detection. This innovative approach can benefit protease detection across various complex environments. The approach can be adapted to any protease as long as a peptide substrate can be conjugated to HS-PEGs and the dissociation capacity of the resulting conjugate can only be restored by the proteolytic activity.

In Example 2, computational methods were used to better understand the mechanism of this reversible aggregation. The short cationic peptide has a steric bulk that maintains some separation distance between the AuNPs thus preventing runaway attractive VdW attraction. We used an all-peptide strategy that is simpler and requires no PEG-peptide couplings. We demonstrated that charge, hydrophilicity, peptide length, and ligand architecture can impact on the dissociation efficiency. Finally, we constructed a practical sensor that is made of the optimized dissociation domain with a biomolecular recognition element of SARS-CoV-2 main protease i.e., M^{pro}.^{17,18} After protease cleavage, released peptides successfully provided a rapid color readout of M^{pro} with a limit of detection (LoD) of 12.3 nM in saliva. Furthermore, our dissociation peptide can dissociate AuNP aggregates in various matrixes including 100% human urine, plasma, and seawater, and can be applied to other types of plasmonic nanoparticles, for example silver (Ag) or platinum (Pt).

Example 2 describes the peptide-driven dissociation of plasmonic assemblies as a response to M^{pro} detection of SARS-CoV-2. This exemplary strategy eliminated the need for surface modifications of AuNPs and complex couplings (for example, PEG-peptide) for protease sensing. Both computational and experimental methods were used to understand reversible aggregation by a short cationic RRK peptide. Using 19 different peptide sequences, we verified that the dissociation capacity relies on hydrophilicity, charge density, ligand architecture, and steric distance. After incorporating the dissociation domain with an M^{pro} cleavage site, a colorimetric assay using UV-vis spectroscopy was tested to confirm the reproducibility and applicability of our platform for M^{pro} detection.

With optimized peptide sequence, this exemplary dissociation strategy successfully produced a distinct optical signal as a function of the released peptides by M^{pro} cleavage with a detection limit of 12.3 nM in saliva. The dissociation-screening site at C terminus enhances proteolytic cleavage (around 3-fold)³² and prevents false positives. Inhibitor assay and specificity test further confirmed the critical role of M^{pro} in dissociation process, showing no non-specific activation. The dissociated AuNPs maintained high colloidal stability in extreme conditions, and this exemplary dissociation strategy can be applied to other types of plasmonic materials such as silver. We further demonstrated that this exemplary dissociation strategy can be less interrupted by matrixes such as human plasma, urine, and seawater. This exemplary peptide-driven dissociation strategy holds significant promise in various fields such as colloidal science, biochemistry, and plasmonic biosensors with diverse applications ranging from disease diagnosis and drug detection to environmental monitoring.

### Products of Manufacture and Kits

Provided are products of manufacture and kits for practicing methods as provided herein; and optionally, products of manufacture and kits can further comprise instructions for practicing methods as provided herein. In alternative embodiments, the products of manufacture comprise (a) a compound X capable of making or forming into reversibly aggregated (or substantially reversibly aggregated) nanoparticles (NPs); and (b) a compound Z conjugated to a poly(ethylene glycol) (Z-PEGₓ), or a plurality of poly(ethylene glycol) (Z-PEGₓs), wherein X is an integer 1, 2, 3, 4, 5, 6, 7, 8, 9, 10, 11, 12, 13, 14 or 15, or X is an integer between about 1 and 40, or between about 2 and 30, or compound Z is conjugated to a peptide.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: a Di-Arginine Additive for Dissociation of Gold Nanoparticle Aggregates: a Matrix-Insensitive Approach with Applications in Protease Detection

This example demonstrates that methods and compositions as provided herein using the exemplary embodiments as described herein are effective in detecting a protease in any biological fluid.

Described herein is date demonstrating the reversible aggregation of gold nanoparticle (AuNPs) assemblies via a di-arginine peptide additive and thiolated PEGs (HS-PEGs). The AuNPs were first aggregated by attractive forces between the citrate-capped surface and the arginine side chains. We found that HS-PEG thiol group has higher affinity for the AuNPs surface, thus leading to redispersion and colloidal stability. In turn, there was a robust and obvious color change due to on/off plasmonic coupling. The assemblies' dissociation was directly related to the HS-PEG structural properties such as their size or charge. As an example, HS-PEGs with a molecular weight below 1 kDa could dissociate 100% of the assemblies and restore the exact optical properties of the initial AuNPs suspension (prior to the assembly). Surprisingly, the dissociation capacity of HS-PEGs was not affected by the composition of the operating medium and could be performed in complex matrices such as plasma, saliva, bile, urine, cell lysates or even sea water. The high affinity of thiols for the gold surface encompasses by far the one of endogenous molecules and is thus favorized. Moreover, starting with AuNPs already aggregated ensured the absence of background signal as the dissociation of the assemblies was far from spontaneous. Remarkably, it was possible to dry the AuNPs assemblies and to solubilize them back with HS-PEGs, improving the colorimetric signal generation. We used this system for protease sensing in biological fluid. Trypsin was chosen as model enzyme and highly positively charged peptides were conjugated to HS-PEG molecules as cleavage substrate. The increase of positive charge of the HS-PEG-peptide conjugate quenched the dissociation capacity of the HS-PEG molecules which could only be restored by the proteolytic cleavage. Picomolar limit of detection was obtained as well as the detection in saliva or urine.

Here, we show a novel process of reversible aggregation of AuNPs-citrate for alternative sensing strategies. Reversible aggregation of nanoparticles is challenging because, according to the Derjaguin-Landau-Verwey-Overbeek (DLVO) theory,²¹ the particles can be trapped in a deep energetic minima during the aggregation, thus transforming the aggregates into larger insoluble materials that can be only slightly dissociated by aggressive sonication; their optical properties cannot be recovered.²² While many in the community have shown reversible assembly of a small number of nanoparticles, this usually requires sophisticated coating of the AuNPs-citrate surface with responsive polymers,^{23,24} DNA strands^{25,26} or other organic ligands.^{27,28} However, we are unaware of work describing the assembly of large amount of AuNPs-citrate into macroscopic aggregates that can easily be dissociated without the need for prior surface modifications.

Exemplary systems as provided herein use only a di-arginine additive that causes the aggregation of AuNPs and thiolated poly(ethylene glycol) (HS-PEGs) for the dissociation of the assemblies (Scheme 1, illustrated in FIG. 1). The system works through differences in affinity of the surface ligands: the introduction of HS-PEGs leads to redispersion of the AuNPs due to the higher affinity of the thiol for the gold surface than the arginine or citrate. In turn, there is a plasmonic color change. Starting with assembled AuNPs allows for insight on the colorimetric signal. Surprisingly, the redispersion can be performed over a wide variety of solvents including human plasma, serum, saliva, and even bile. After characterizing the ligands and HS-PEGs best suited for this reaction, we deployed it for colorimetric sensing of proteases via a cleave PEG-peptide conjugate. Although the grafting of HS-PEGs molecules on dispersed AuNPs has been widely reported in the literature, the dissociation of AuNPs assemblies with HS-PEGs has never been studied before, especially for the detection of proteases.

### Results and Discussion

Formation of AuNP assemblies. In this study, we investigated the possibility of forming convenient and reversible AuNPs assemblies using only elementary 15 nm AuNPs-citrate suspended in water and short peptides without the need for a complex surface modification. Peptides were used because they are relatively bulky and thus their steric prevented the particles from entering a permanent aggregated state. Arginine in particular can strongly interact with citrate anions via electrostatic interactions. A dipeptide containing two repetitions of arginine (Arg-Arg or RR) was thus used to interact with multiple particles at the same time and induce assembly.

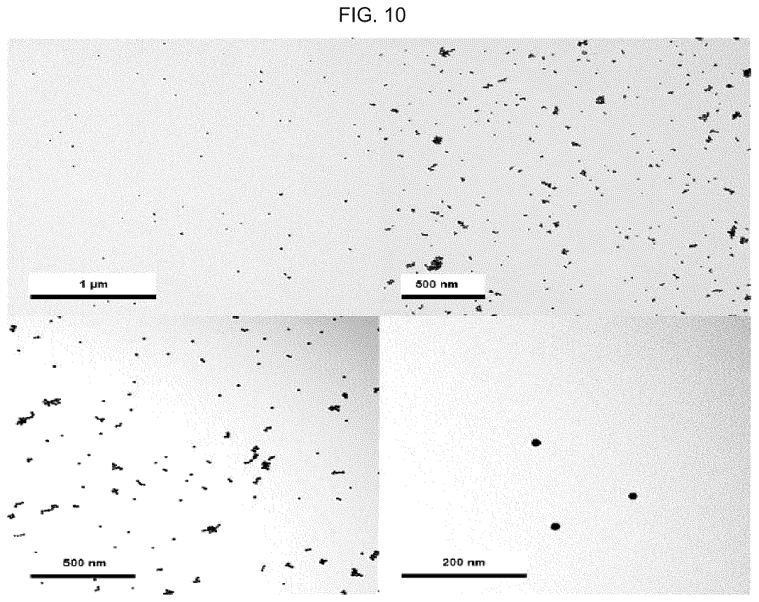

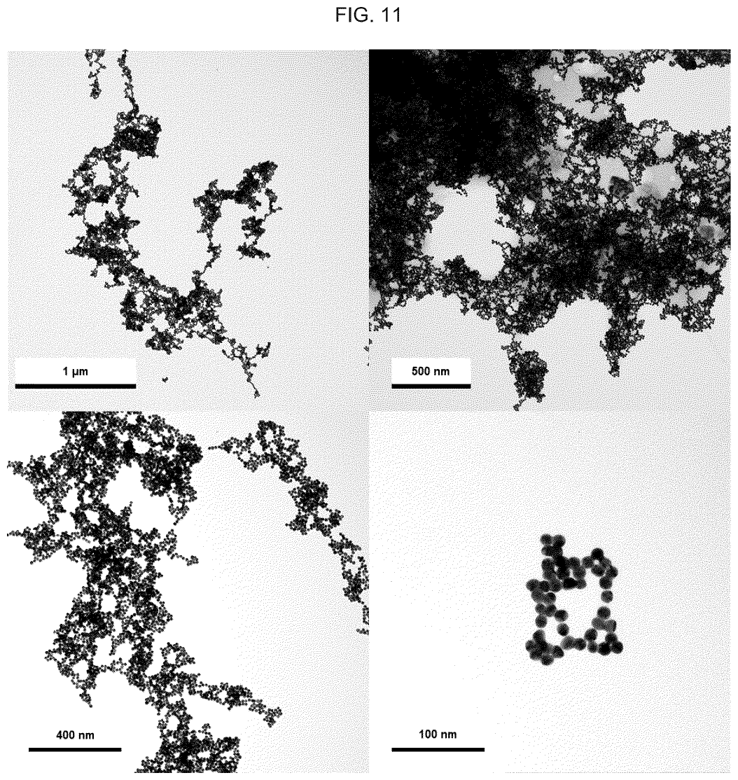

Transmission electronic microscopy (TEM) revealed that the addition of excess (10⁴ equiv.) of di-arginine peptide (Arg-Arg) to an aqueous suspension of AuNPs-citrate led to bulky assemblies and no dispersed AuNPs were observed (FIG. 1A vs 1B). Multispectral advanced nanoparticles tracking analysis (MANTA) was then used to confirm the size increase: The initial blue scattering corresponding to a hydrodynamic diameter of 40±25 nm (FIG. 1C) transformed immediately into red scattering corresponding to a hydrodynamic diameter of approximately 467±120 nm (FIG. 1D). Importantly, the count of particles decreased by more than 90%—from 2.5×10⁵/mL to 2×10⁴/mL, thus confirming that the vast majority of the AuNPs-citrate were aggregated. TEM images at different magnifications are seen in the supporting information (FIG. 10 and FIG. 11).

The peptide-induced assembly of AuNPs-citrate strongly impacted the optical properties of the colloid. An immediate modification of the LSPR band of the particles was observed proportional to the peptide concentration. The absorbance at λₘₐₓ decreased, and a new absorption peak increased at 700 nm (FIG. 1E). Such deformation of the LSPR band led to a change of color of the suspensions turning from bright red to blue (FIGS. 1E, insets). The ratio of the absorbance at 520 nm over 700 nm were then used to characterized the AuNPs assemblies through all this study. Interestingly, the density of Arg-Arg leading to the maximal ratio of absorbance was approximately 1.8 Arg-Arg-/nm², which corresponds approximately to the maximal density of Arg-Arg that could be carried by one AuNPs (1.6 Arg-Arg/nm²) considering a footprint for the peptide of 0.63 nm² (see http://biotools.nubic.northwestern.edu/proteincalc.html).

Arg-Arg was chosen to promote the AuNPs-citrate assemblies because it is the minimal sequence that can induce the assembly. The reversibility of this exemplary system is based on the replacement of citrate and peptide layers by PEG, and it was thus crucial to remove the peptides from the AuNPs surface. While a single arginine could not induce any form of assembly, peptides containing more than two arginine had a higher affinity for the AuNPs-citrate than Arg-Arg and were thus discarded (FIG. 1F). As a control, we synthesized a peptide containing two arginine but spaced by three glycine (RGGGR) to decrease the charge density and thus lower the affinity of the peptide for the AuNPs-citrate. However, RGGGR had a higher affinity for the particles than RR (Arg-Arg) and was also discarded.

Despite its short length, Arg-Arg was sufficient to protect the AuNPs from degradation, and the resulting assemblies were highly stable over time. FIG. 1G shows barely no difference between the LSPR band of pristine and 120-day-old Arg-Arg-AuNPs. This provides another demonstration of the high stability of the assemblies was performed with their drying and resuspension in pure water using only 5 seconds of sonication without any degradation or loss of particles (FIG. 1H).

FIG. 1. Peptide-induced assembly of AuNPs-citrate. (FIGS. 1A, B, C and D) TEM images and multispectral advanced nanoparticle tracking analysis (MANTA)²⁹ image and size distribution of AuNPs-citrate before (A and C) or 10 minutes after the addition of 10 μM of Arg-Arg (B and D). MANTA records images of the particles' light scattering via three differently colored lasers (for example, blue, green, and red); the scattering depends on the particle's size. MANTA counts the nanoparticles and calculates their size. (E) Modification of the optical properties of AuNPs-citrate during the assembly with Arg-Arg. Insets show the pictures of the samples for concentrations of Arg-Arg of 0, 1, 2, 5 and 10 μM. (F) Ratio of the absorbances Abs.520 nm/Abs.700 nm of AuNPs-citrate as a function of peptides concentration. (G) UV-Vis spectra of Arg-Arg-AuNPs 1 day or 120 days old. (H) Pictures of Arg-Arg-AuNPs dried and resuspended in pure water after 5 seconds of sonication.

To demonstrate the versatility of this exemplary system, this study was reproduced with 40 nm AuNPs-citrate commercially available from Nanocomposix (supporting information Section 7).

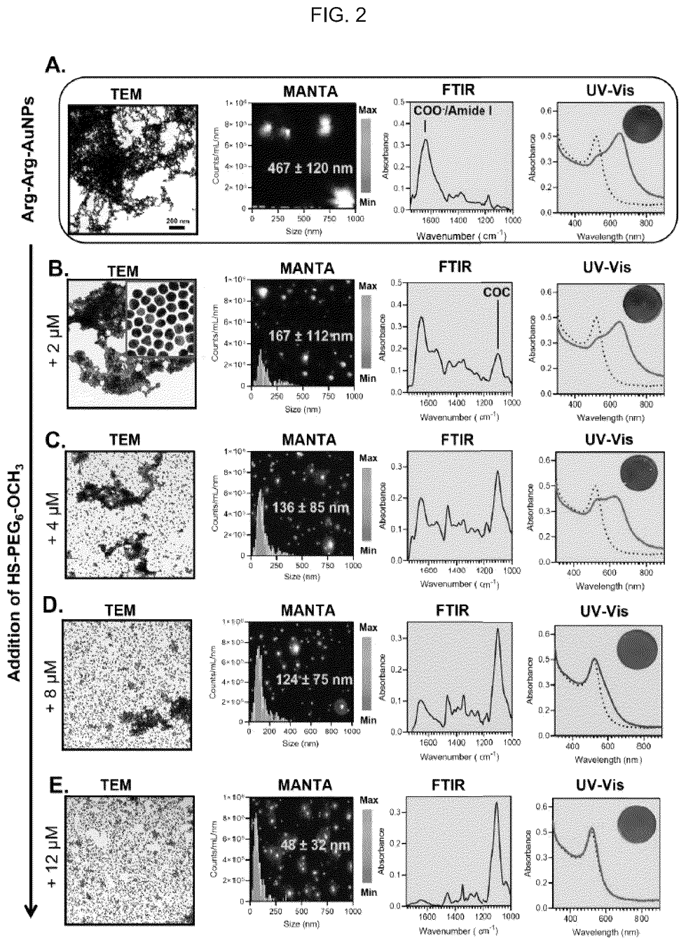

Dissociation of the AuNPs assemblies. The proof-of-concept of the assembly reversibility was demonstrated with a polyethylene glycol (PEG) system containing six repetitions capped with a methoxy group and a thiol at the other end (HS-PEG₆-OCH₃). HS-PEG₆-OCH₃ was chosen because its grafting on AuNPs ensure a high colloidal stability of these latter. Indeed, the grafting of thiolated PEGs has been widely reported in the literature via the formation of a strong Au—S bond conferring to the particles a high colloidal stability due to a combination of hydrophilicity and steric hindrance.³⁰⁻³² The Au—S bond being stronger than the arginine-citrate, arginine-gold or even citrate-gold interactions, HS-PEG₆-CH₃ was expected to be capable of progressively replacing the arginine/citrate layers at the gold surface, disrupting the electrostatic network, and eventually dispersing the AuNPs (Scheme 1). Briefly, aqueous suspensions Arg-Arg-AuNPs assemblies were exposed to increasing concentrations of HS-PEG₆-OCH₃, and the dissociation of the assemblies was characterized by TEM, MANTA, FTIR and UV-Vis spectroscopies (FIG. 2).

Prior to any addition of HS-PEGs, the dense and large aggregates of Arg-Arg-AuNPs present only ATR-FTIR signals coming from citrate and/or Arg-Arg as well as COO⁻ᵥₐₛ and/or amide band I around 1650 cm⁻¹, respectively. The color of the sample was bright blue because the LSPR band was strongly red-shifted (FIG. 2A).

FIG. 2. Characterization of the dissociation of AuNPs assemblies with HS-PEG₆-OCH₃. From left to right, TEM*, MANTA, ATR-FTIR**, and UV-Vis*** spectroscopy analysis of Arg-Arg-AuNPs (A) before or 10 minutes after the addition of (B) 2 μM, (C) 4 μM, (D) 8 μM, and (E) 12 μM of HS-PEG₆-OCH₃. *All the images are at the same magnification. **All samples were cleaned by centrifugation from non-bound molecules (HS-PEGs, citrate or Arg-Arg) and the FTIR spectrum corresponds only to molecules attached to the gold surface. *** Black dashed line shows the LSPR band of the initial AuNPs-citrate.

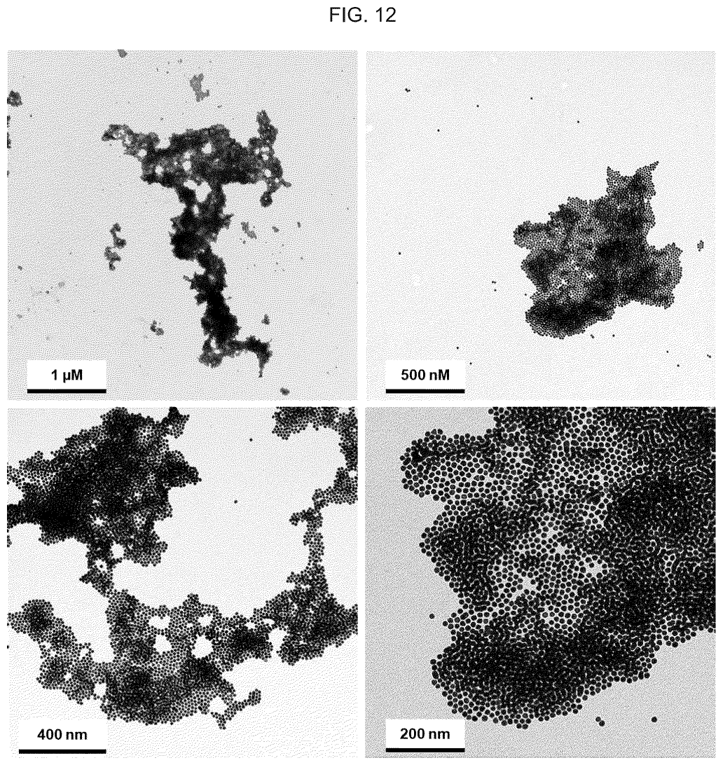

After the addition of 2 μM of HS-PEG₆-OCH₃, however, the distance between the AuNPs in the aggregates increased and the size of the aggregates decreased from 467±120 nm to 167±112 nm. Interestingly, ATR-FTIR spectroscopy showed an increase in the absorbance at 1100 cm⁻¹ corresponding to the C—O—C stretching of the PEG chain and a decrease in the absorbance around 1650 cm⁻¹. This indicates that few HS-PEG₆-OCH₃ were grafted onto the AuNPs surface, which explains the increase in the interparticle distance. The decrease in the size of the assembly led to a color change from blue to purple (FIG. 2B and FIG. 12).

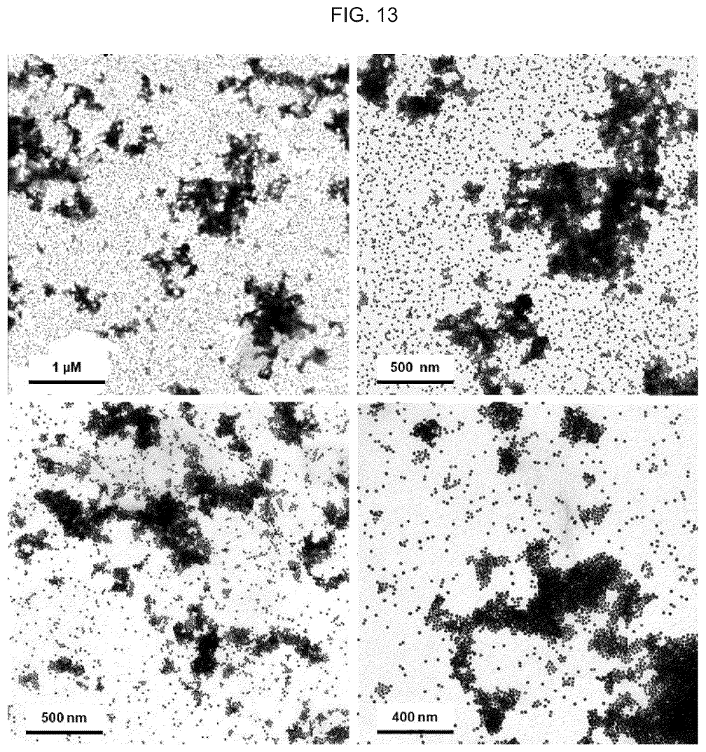

FIGS. 2C and 2D show the results of the addition of 4 μM and 8 μM of HS-PEG₆-OCH₃, respectively. More AuNPs detached from the aggregates and became monodisperse with higher concentrations of HS-PEG₆-OCH₃ (FIG. 12 and FIG. 13). Accordingly, the average size of the aggregates decreased to 136±85 nm and 124±75 nm for 4 μM and 8 μM, respectively. Moreover, the intensity of the ATR-FTIR signals of the PEG chain increased and the signal of the citrate/Arg-Arg decreased, thus confirming the expansion of the grafting density of HS-PEG₆-OCH₃. The LSPR band of the colloid red-shifted due to dissociation: The solution color were purple-pink and wine red at 4 μM and 8 μM, respectively.

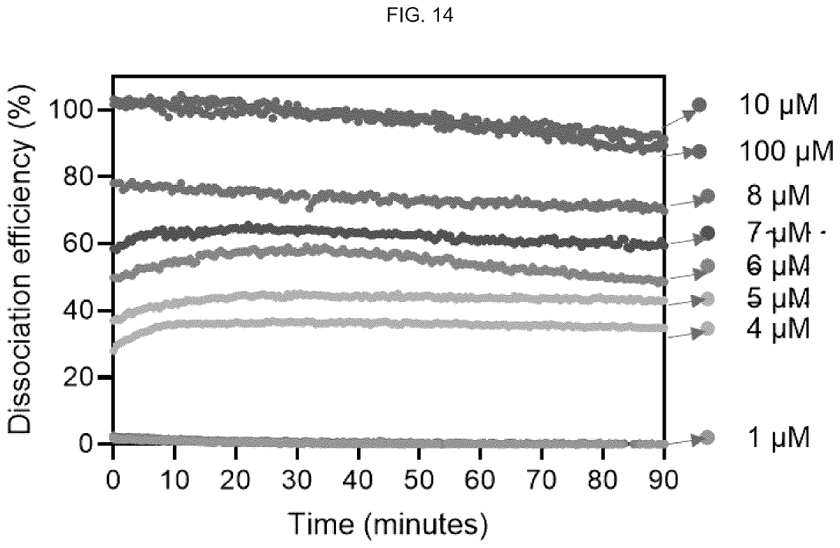

At 12 μM HS-PEG₆-OCH₃ and above, aggregates were no longer seen in TEM, and MANTA measured a mean size of 48±25 nm. ATR-FTIR showed only absorbance of the C—O—C signal with limited citrate/Arg-Arg signals, thus indicating that the citrate/arginine layer was completely removed from the surface for the benefit of HS-PEG₆-OCH₃. The presence of a PEG layer around the AuNPs explains the difference in the hydrodynamic diameter versus the initial AuNPs-citrate (48±32 nm vs 40±25 nm). Remarkably, the LSPR band of the dissociated AuNPs was identical to the AuNPs-citrate: The color of the sample returned to its initial bright red color. This suggests that 100% of the aggregates were dissociated and that all AuNPs were detached and dispersed. For the remainder of this study, the efficiency of the dissociation will be characterized by the percentage of dissociation (%) as measured by UV-Vis spectroscopy (comparison between the LSPR band after dissociation versus that from AuNPs-citrate). See the experimental section in the supporting information for more details. Importantly, a concentration of 12 μM corresponds to a density of approximately 4 HS-PEG₆-OCH₃/nm². This finding is particularly interesting because the typical grafting density of HS-PEG₆-OCH₃ on dispersed AuNPs is reported to be between 3.5 and 4 HS-PEG₆-OCH₃/nm².³³ This implies that no excess of HS-PEGs was needed to dissociate the entire assembly-only enough to cover all the gold surface. It is worth noting that the kinetics of the dissociation was almost instantaneous (equilibrium reached within a minute) (FIG. 14). A video of the dissociation of the AuNPs assemblies is available online.

These findings suggest that HS-PEG₆-OCH₃ can penetrate the assembly and graft onto the AuNPs surface, thus displacing the citrate and arginine layer. The particle becomes sterically stabilized and water-soluble when a sufficient amount of HS-PEG₆-OCH₃ is grafted on the particle surface; thus, the particle detaches from the assembly. When the concentration of HS-PEG₆-OCH₃ is sufficiently high to cover all of the particle surface, all assemblies dissociate, and the optical properties of the AuNPs are restored to those of the initial dispersed AuNPs-citrate. This phenomenon is possible only because (i) the AuNPs are not trapped in permanent aggregated state and (ii) HS-PEG₆-OCH₃ can replace the citrate/arginine layers due to the covalent grafting onto the gold surface.

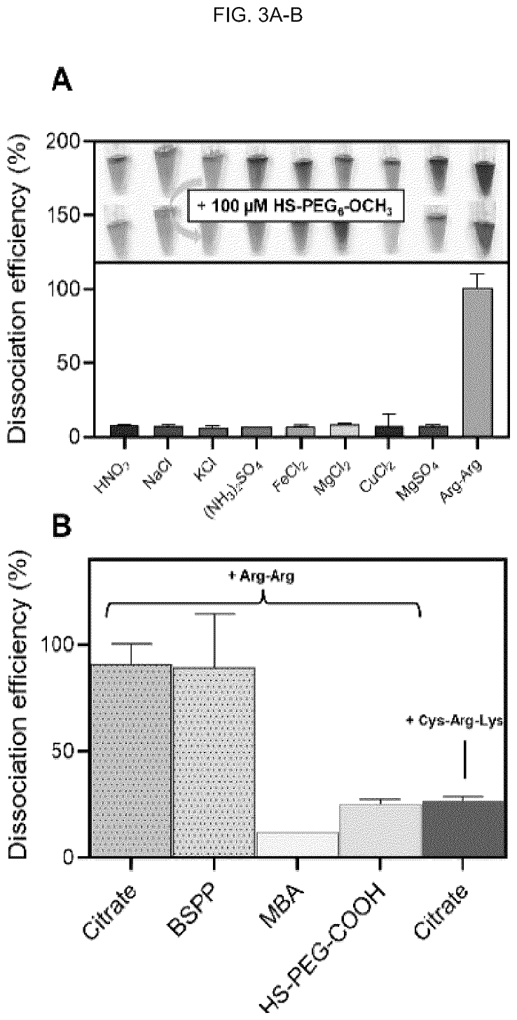

To demonstrate the necessity of these two features, multiple control experiments were conducted. First, peptide-free conditions were used to promote the assembly of AuNPs-citrate and its dissociation with HS-PEG₆-OCH₃ was evaluated (FIG. 3A). Here, salts were added to AuNPs-citrate to induce aggregation via disruption of the electrostatic repulsion forces between the particles. Even the addition of a high concentration of HS-PEG₆-OCH₃ (greater than (>) 100 μM) could not dissociate the assemblies and the color of the samples remained dull blue/grey. Unlike the peptide Arg-Arg, these ions cannot sterically prevent the AuNPs from falling in an irreversible aggregated state.

FIG. 3. Dissociation efficiency of HS-PEG₆-OCH₃ for alternative assembly conditions or coating ligands. (A) Dissociation efficiency of 100 μM of HS-PEG₆-OCH₃ added to AuNPs-citrate assembled in peptide-free conditions. (B) Dissociation efficiency of 100 μM of HS-PEG₆-OCH₃ added to either AuNPs-citrate, AuNPs-BSPP, AuNPs-MBA, or AuNPs-S-PEG-COOH assembled with 10 μM of Arg-Arg or AuNPs-citrate assembled with 10 μM of Cys-Arg-Lys peptide.

AuNPs with different coating ligands were also evaluated and compared to citrate: bis(p-sulfonatophenyl)phenylphosphine (BSPP), mercaptobenzoic acid (MBA), or thiolated PEG-COOH (HS-PEG-COOH; Mw=634 g·mol⁻¹). Similar to the AuNPs-citrate, the presence of Arg-Arg led to the assembly of AuNPs-BSPP, AuNPs-MBA, and AuNPs-S-PEG-COOH. However, only the AuNPs-BSPP could be dissociated; assemblies of AuNPs-MBA or AuNPs-S-PEG-COOH were irreversible even in the presence of high concentrations (greater than (>) 100 μM) of HS-PEG₆-OCH₃ (FIG. 3B). This is because MBA and HS-PEG-COOH make covalent Au—S bonds with the gold surface that prevent subsequent grafting of HS-PEG₆-OCH₃. In contrast, BSPP, like citrate, is only physiosorbed onto the gold surface and can be easily displaced by HS-PEG₆-OCH₃, thus making the assembly reversible.

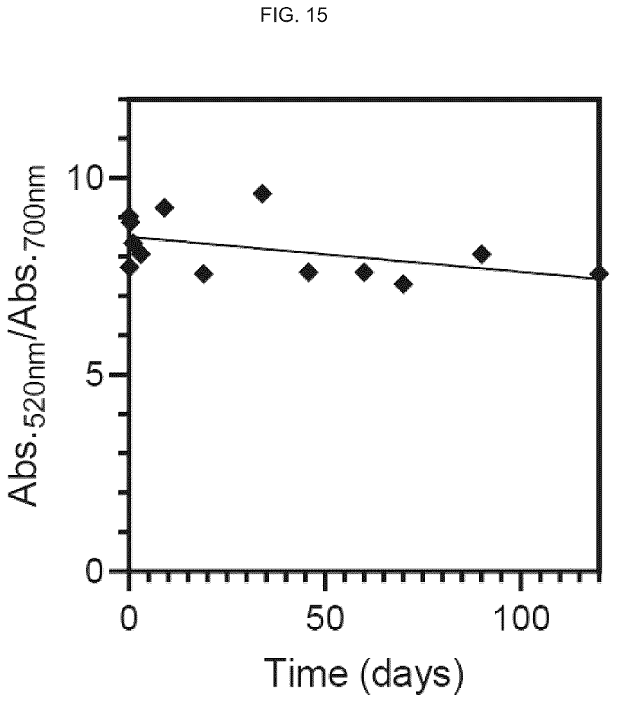

Finally, an alternative control peptide was used for assembly (Cys-Arg-Lys). This peptide could aggregate the AuNPs-citrate but the assembly was irreversible (FIG. 3B). This is because this peptide can make a covalent Au—S bond with the gold surface via the cysteine, thus hindering the grafting of HS-PEG₆-OCH₃ on the AuNPs surface and preventing dissociation. These control experiments show that the coating ligands as well as the aggregation peptide need to be weakly adsorbed onto the gold surface to facilitate aggregation and dissociation. Importantly, as the aggregates do not degrade over time: their dissociation was still possible even 120 days after their formation without a significant difference in the percent dissociation (FIG. 15).

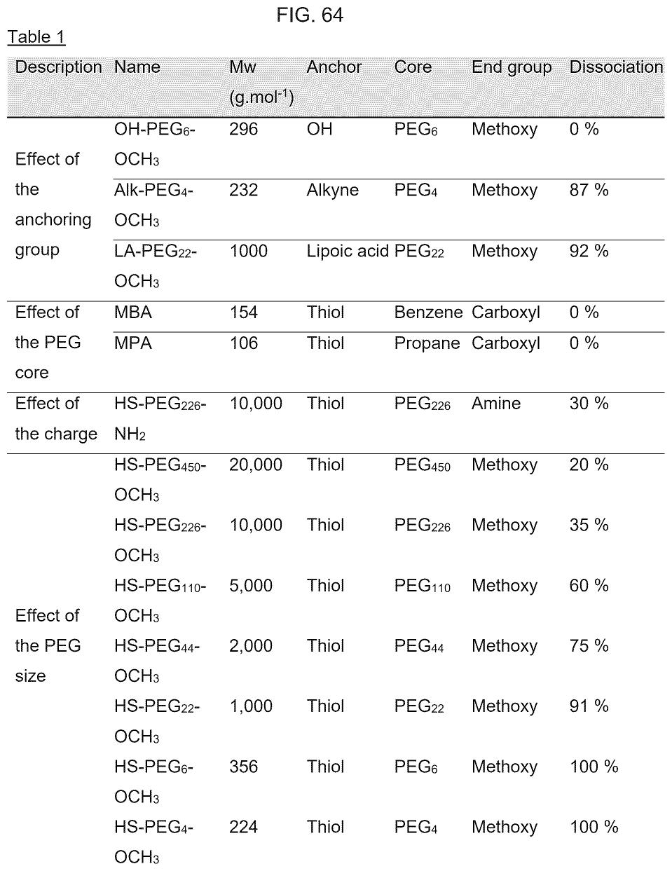

Effect of the PEGs structure on the dissociation capacity. The impact of PEGs structure on dissociation capacity was studied next, i.e., ligands differing either by their size, anchoring group, core, or charge, see FIG. 64 for Table 1.

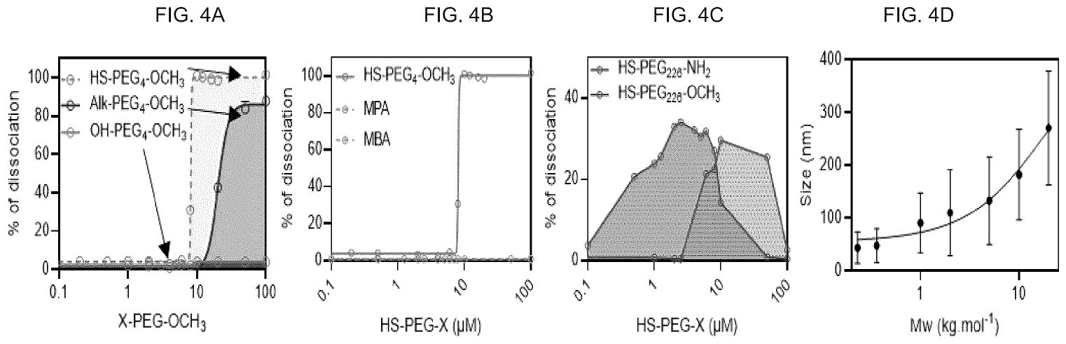

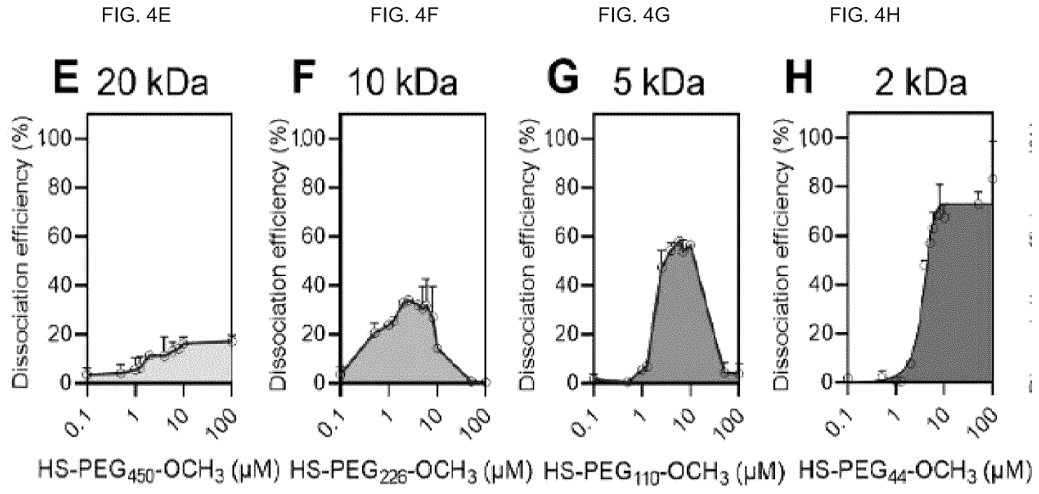

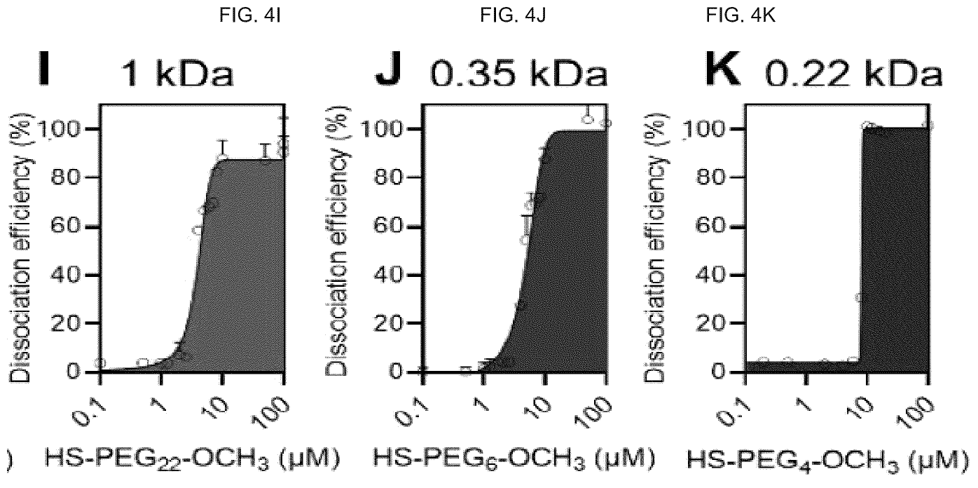

First, to confirm that the grafting of the PEG on the AuNPs is critical to dissociation, methoxy PEG molecules carrying different anchoring groups (x-PEG-OCH₃) were studied. FIG. 4A shows that the dissociation of Arg-Arg-AuNPs assemblies is no longer possible when the thiol group (HS-PEG₄-OCH₃) is replaced by a hydroxy group (OH-PEG₄-OCH₃) even at high concentrations (>100 μM): This is because OH-PEG₄-OCH₃ cannot bind covalently to the gold surface. Positive controls involving alkyne (Alk-PEG₄-OCH₃) and lipoic acid (LA-PEG₂₂-OCH₃) as anchoring groups were investigated because these two chemical groups, like thiol groups, can form a covalent bond with the gold atoms at the AuNPs surface.^{34,35} Thus, as expected, the addition of Alk-PEG₄-OCH₃ or LA-PEG₂₂-OCH₃ led to the dissociation of the AuNPs assemblies (FIG. 4B). Interestingly, while LA-PEG₂₂-OCH₃ had a similar dissociation efficiency than its thiolated counterpart (HS-PEG₂₂-OCH₃), the one of Alk-PEG₄-OCH₃ was lower compared to HS-PEG₄-OCH₃. It can be explained by the fact that alkynes form a less labile strong bond with the surface compared to thiols. Overall, the results confirm the necessity for the dissociating ligands to be capable of grafting onto the AuNPs surface.

FIG. 4. Effect of the PEG properties on the dissociation efficiency. (A) Dissociation efficiency of X-PEG-OCH₃ molecules carrying various anchoring groups added to Arg-Arg-AuNPs. X=thiol (HS), hydroxy (OH), lipoic acid (LA) or Alkyne (Alk). (B) Dissociation efficiency of HS-PEG₄-OCH₃, MBA, or MPA added to Arg-Arg-AuNPs. (C) Dissociation efficiency of HS-PEG₂₂₆-OCH₃ compared to HS-PEG₂₂₆-NH₂ added to Arg-Arg-AuNPs. (D) Size of the assembly 20 minutes after the addition of HS-PEGₓ-OCH₃ to Arg-Arg-AuNPs of different molecular weight (20, 10, 5, 2, 1, 0.35 and 0.22 kDa). (E), (F), (G), (H), (I), (J) and (K). Dissociation efficiency of HS-PEGₓ-OCH₃ concentration with different molecular weight added to Arg-Arg-AuNPs.

Non-PEG ligands such as mercaptobenzoic acid (MBA) and mercaptopropionic acid (MPA) were investigated next: Interestingly, none could dissociate the aggregates despite the presence of a thiol group in their structures (FIG. 4B). The flexibility and hydrophilicity of the PEG chain is likely crucial to make the AuNPs water-soluble and detach them from the bulky and hydrophobic aggregate.

Finally, we demonstrated that the charge and the size of the PEG molecules have a strong impact on their capacity to dissociate AuNPs aggregates. FIG. 4C shows that the dissociation of HS-PEG₂₂₆-OCH₃ is strongly affected when the methoxy group is replaced by an amine group (net charge=+1). The effect of the size of the PEG molecule was then studied by using various HS-PEGₓ-OCH₃ of different molecular weights ranging from 20 kDa to 0.22 kDa. FIG. 4D shows that the size of the assemblies (measured by MANTA) is inversely proportional to the size of the HS-PEGₓ-OCH₃. This result was confirmed by UV-Vis spectroscopy: HS-PEGₓ-OCH₃ with a molecular weight of 20, 10, 5 and 2 kDa, could only dissociate a maximum of 20%, 35%, 60%, and 70% of the AuNPs assemblies, respectively (FIGS. 4E, 4F, 4G and 4H). However, the 1 kDa HS-PEGₓ-OCH₃ offered 80% dissociation; this value reached 100% for HS-PEGₓ-OCH₃ with a molecular weight of 0.35 kDa or smaller (FIGS. 4I, 4J and 4K). These data illustrate that the dissociation efficiency of PEG is directly proportional to PEG size. Large HS-PEGs likely cannot reach the interfaces of the aggregated particles while smaller HS-PEGs can. However, the concentration of HS-PEGₓ-OCH₃ needed to initiate the dissociation was proportional to their size because the footprint of HS-PEGs is directly dependent on their size. Thus, larger HS-PEGs cover a larger surface on the AuNPs and less PEGs molecules are needed to complete the coating. This non-exhaustive study reveals that the dissociation of AuNPs assemblies can be directly modulated by tuning the PEGs properties, and it is particularly interesting for the development of sensing strategies.

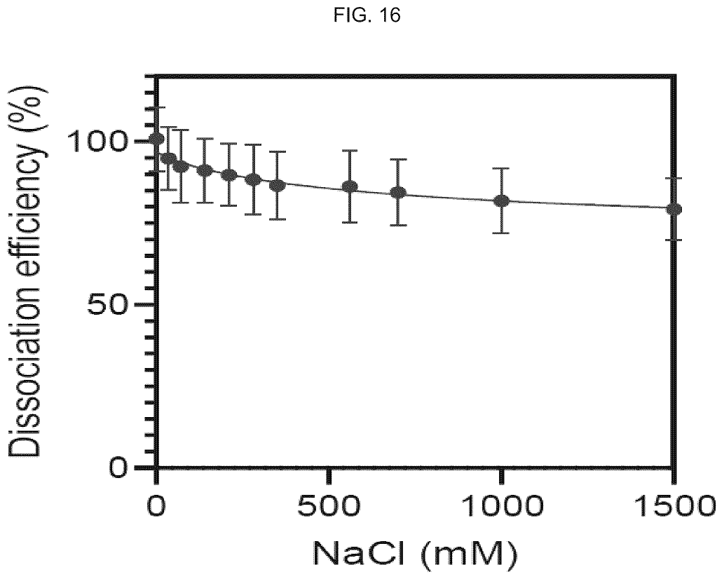

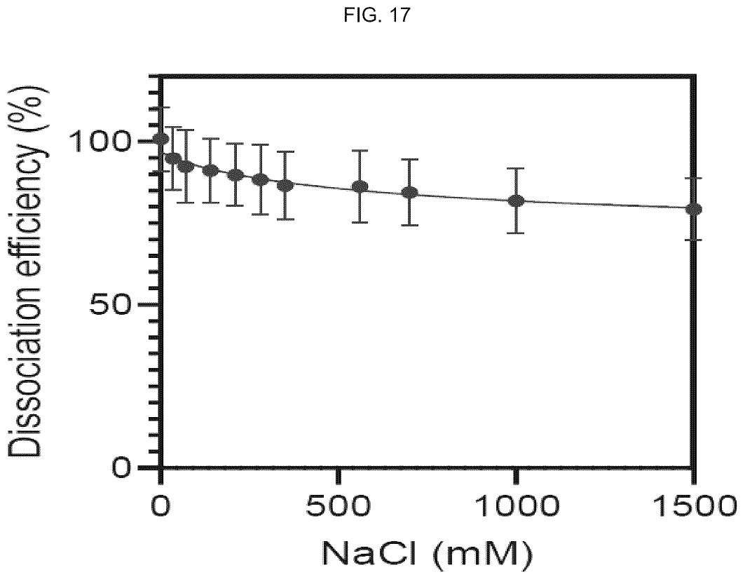

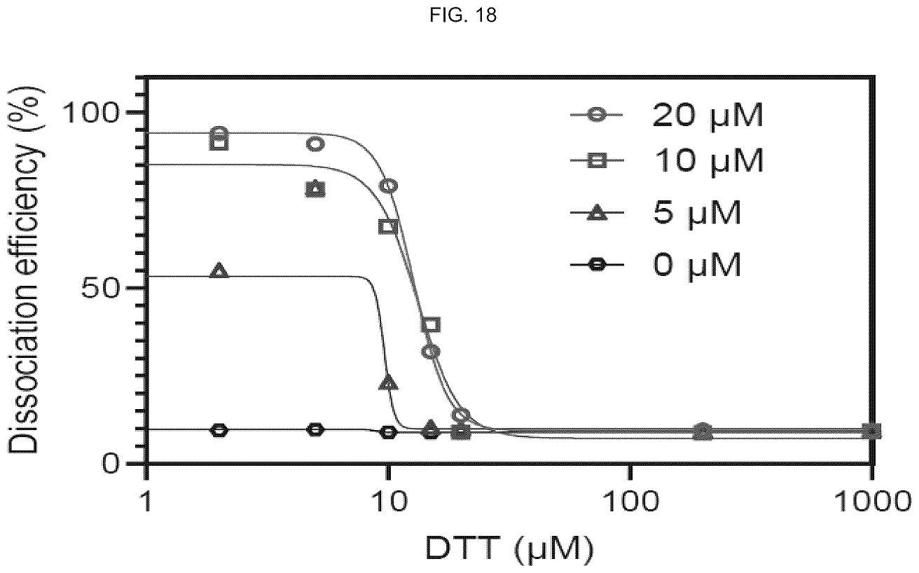

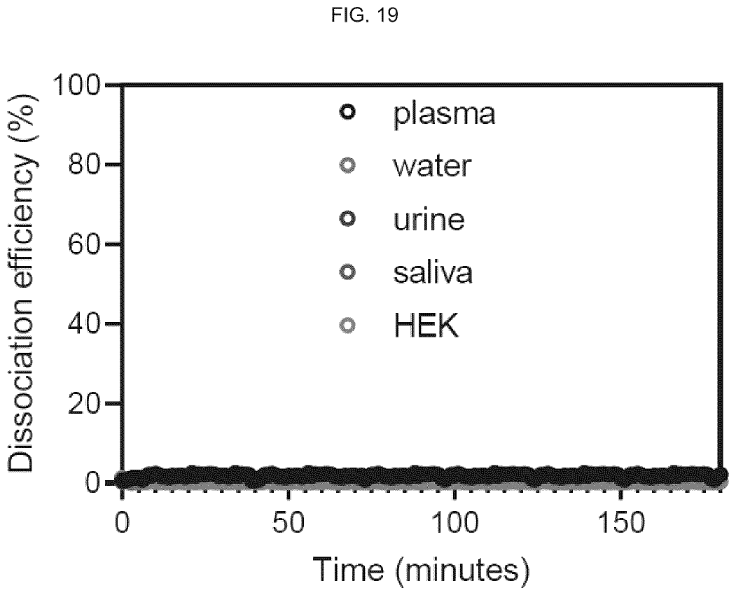

Advantages of exemplary dissociation approaches. This exemplary dissociation approach possesses two major advantages compared to the conventional aggregation-based assay. First, it can operate across various complex samples that is crucial for assay generalizability. Typically, background interferents differ across different sample matrices; thus, it is challenging to obtain an unambiguous colorimetric signal that is insensitive to the matrix composition, particularly for AuNPs aggregation-based assays because the colloids can be unstable in these conditions or because endogenous molecules can prevent the aggregation. The dissociation of the AuNPs assembly is very robust: It is unaffected by high ionic strength (greater than (>) 1 M NaCl) (FIG. 16) or pH extremes (3 or 13) (FIG. 17). Only compounds that can interact with the thiol group can interfere with the dissociation. As an example, dithiothreitol (DTT) quenching is presented in Figure FIG. 18. Thus, we next evaluated dissociation as a function of sample type.

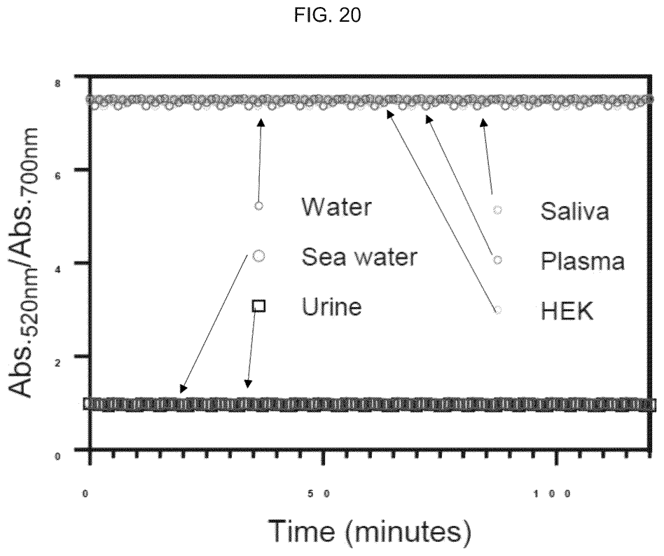

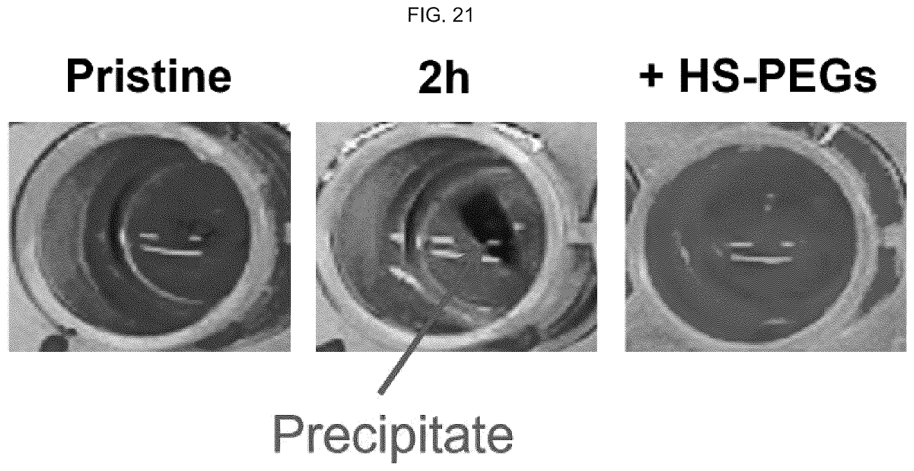

The dissociation of Arg-Arg-AuNPs assemblies was investigated in pooled plasma, pooled urine, pooled saliva, pooled bile, human embryonic kidney (HEK) 293 cell lysates in Dulbecco's modified Eagle's medium (DMEM), and even sea water. Importantly, the matrices by themselves could not dissociate the assemblies even after three hours of incubation (FIG. 19). This is particularly interesting because these complex environments usually produce dramatic background signals. For example, the dispersibility of AuNPs-citrate is strongly impacted when suspended in urine or sea water due to the high ionic strength (see FIG. 19, FIG. 20). The dissociation of Arg-Arg-AuNPs in complex matrices was investigated with 10 μM HS-PEG₆-OCH₃. Typically, concentrated Arg-Arg-AuNPs were suspended in the complex matrix and then HS-PEG₆-OCH₃ was added. The complex matrix represented at least 90% of the total volume. At least 80% of dissociation was obtained in all the matrices including an unambiguous color change from blue to red (FIGS. 5A and 5B).

FIG. 5. Dissociation of AuNPs assemblies in complex matrices. (A) Dissociation efficiency of HS-PEG₆-OCH₃ added to Arg-Arg-AuNPs suspended in various media and (B) the corresponding pictures before and 20 minutes after the addition of 10 μM of HS-PEG₆-OCH₃. The complex medium was approximately 90% of the total volume except for the bile that was 20%. (C) Dissociation of Arg-Arg-AuNPs with HS-PEG₆-OCH₃ compared to the Arg-Arg-induced aggregation of AuNPs-citrate in various complex matrices. Note that HEK/DMEM corresponds to HEK 293 cell lysates coming from suspension of 10⁶ cells in Dulbecco's modified Eagle's media with 10% fetal bovine serum and 1% penicillin-streptomycin. (D) UV-Vis spectra of dried Arg-Arg-AuNPs film after dissociation with aqueous solution of HS-PEG₆-OCH₃ of various of concentrations. Insets show the absorbance at 520 nm as a function of the concentration of HS-PEG₆-OCH₃ and the corresponding pictures.

The re-dispersion of AuNPs assemblies was then compared to the aggregation of dispersed AuNPs-citrate with the Arg-Arg peptide in complex media (FIG. 5C). Even high concentrations of Arg-Arg (greater than (>) 200 μM) could not induce the AuNPs-citrate aggregation in biological fluids because of the numerous endogenous molecules/proteins that shield the electrostatic interactions between the peptide and the particles as well as the formation of a protein corona around the particles.¹⁷ The matrix-insensitive feature of this exemplary dissociation design is explained by the fact that most of the biomolecules found in the complex samples (proteins, phospholipids, nucleotides, etc.) can only stick onto the gold surface via van der Waals forces, electrostatic or hydrophobic interactions, or hydrogen bonds.^{17,36} Thus, the grafting of HS-PEGs is favored because the Au—S bond energy is approximately 10-fold higher than the average hydrogen bond.³⁷ Also, PEGs are commonly used to prevent non-specific adsorption of proteins on AuNPs, and thus the HS-PEGs do not interact much with the interferents; they remain free to bind to the particles.

The second advantage of this exemplary dissociation approach is that the assemblies did not just remain aggregated, they also precipitated, thus increasing the color change between the dissociated and aggregated systems. As an example, FIG. 54A-B shows that Arg-Arg-AuNPs suspended in saliva after 2 hour tend to precipitate, thus leading to a loss of blue color in the suspension while the dissociated particles remain bright red. Interestingly, even if precipitated, the assemblies can still be dissociated with 10 μM of HS-PEG₆-OCH₃.

Remarkably, the HS-PEGs could even solubilize dried films of Arg-Arg-AuNPs (FIG. 5D). Briefly, 50 μL of aqueous solution containing different concentrations of HS-PEGs, ranging from 0 to 70 μM, were added to the dried particles and 20 minutes later, the UV-Vis spectrum of the solution was recorded. In the absence of HS-PEGs, no LSPR band of the particles was observed as no particles detached spontaneously and the solution remained clear. However, in the presence of HS-PEGs, the plasmonic band of the particles was observed proportionally to the HS-PEGs concentration without the need of sonication. The HS-PEGs led to the spontaneous and progressive detachment of the AuNPs from the surface and thus a color change (FIG. 5D, inset). The solubilization of the AuNPs could be thus quantified using only the absorbance at 520 nm in the solution as the LSPR band intensity was linearly proportional to the HS-PEGs concentration. This approach did not work on AuNPs-citrate as they degraded during the drying process—the di-peptide is a crucial additive. Finally, the solubilization of dried Arg-Arg-AuNPs was possible even in biofluids such as saliva, plasma, urine or cell lysates (FIG. 55A-B).

Thus, this exemplary dissociation strategy affords two advantages versus traditional aggregation-based assays: It is insensitive to the composition of the operating medium and the gap of colorimetric signals between dissociated and non-dissociated samples is unambiguous and increases over time, thus enhancing the naked eyes identification.

Protease detection. The charge and size of the HS-PEGs modulate their capacity to dissociate the AuNPs assemblies, and thus we designed a strategy to detect proteases in biological fluids. Trypsin was chosen as a model protease because it possesses a high catalytic efficiency and easily cleaves peptide sequences after arginine or lysine.³⁸ Trypsin is a biomarker of pancreatic cancer and can be found in micromolar concentrations in blood or urine.³⁹

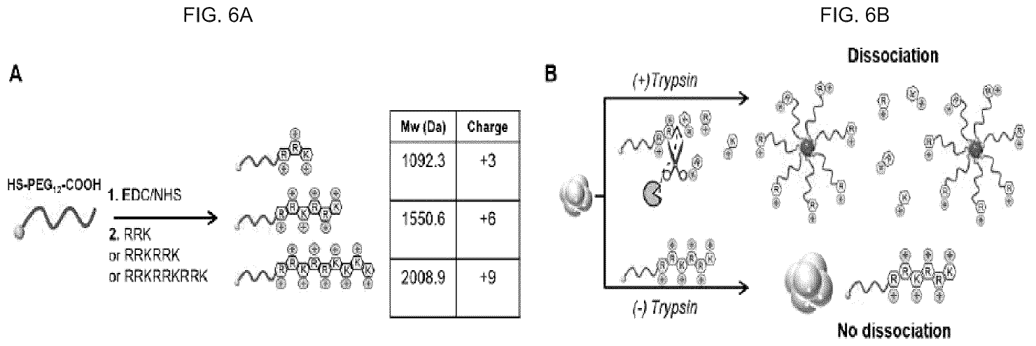

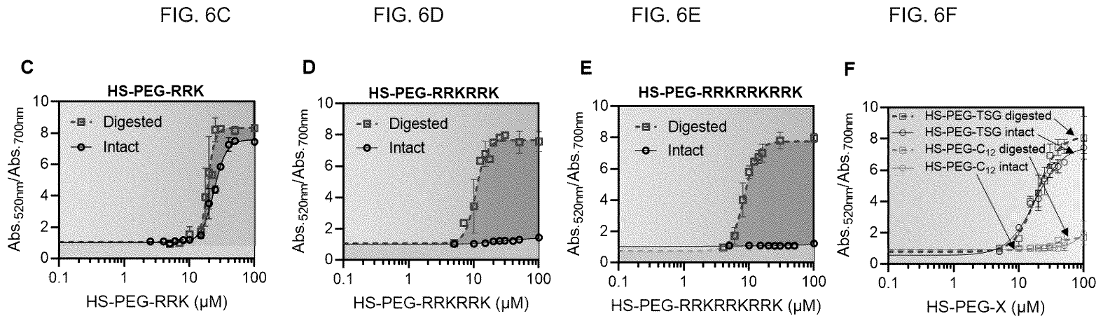

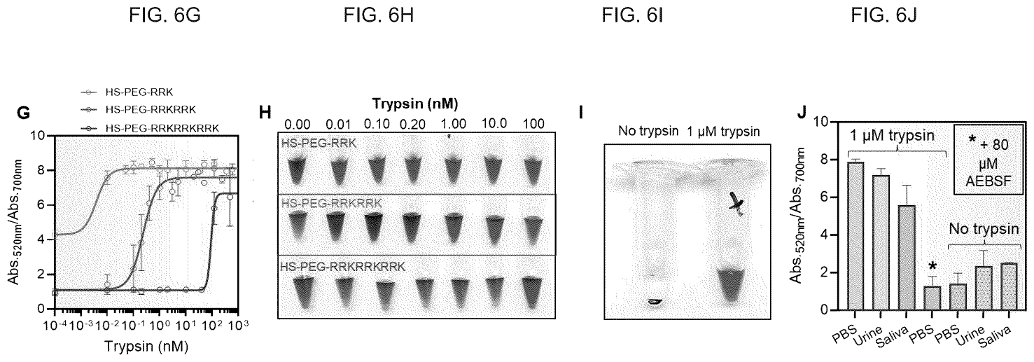

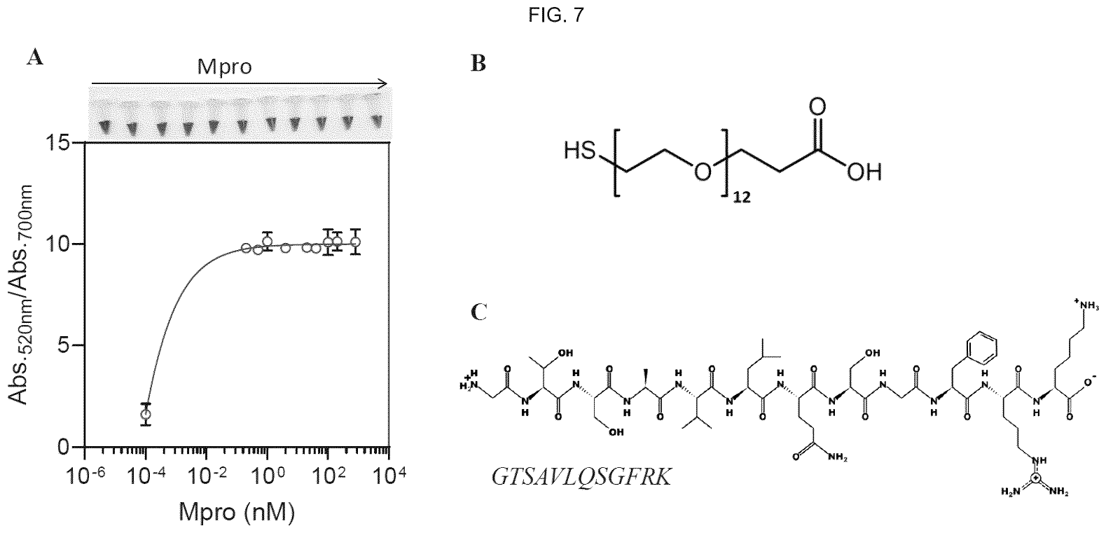

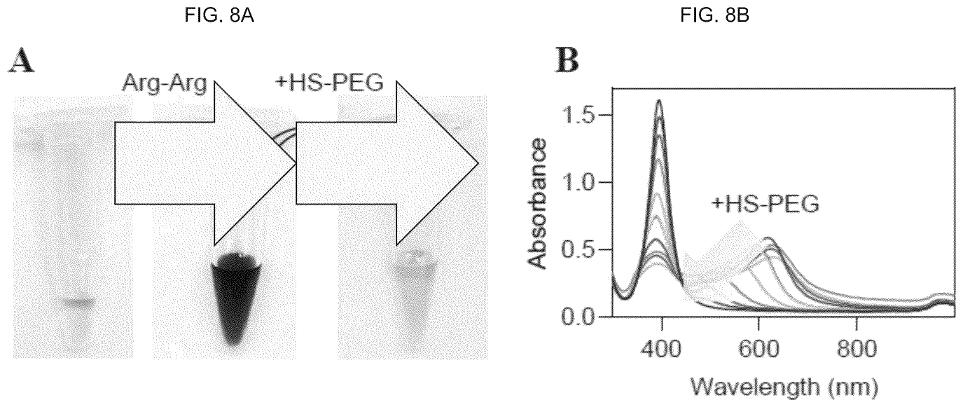

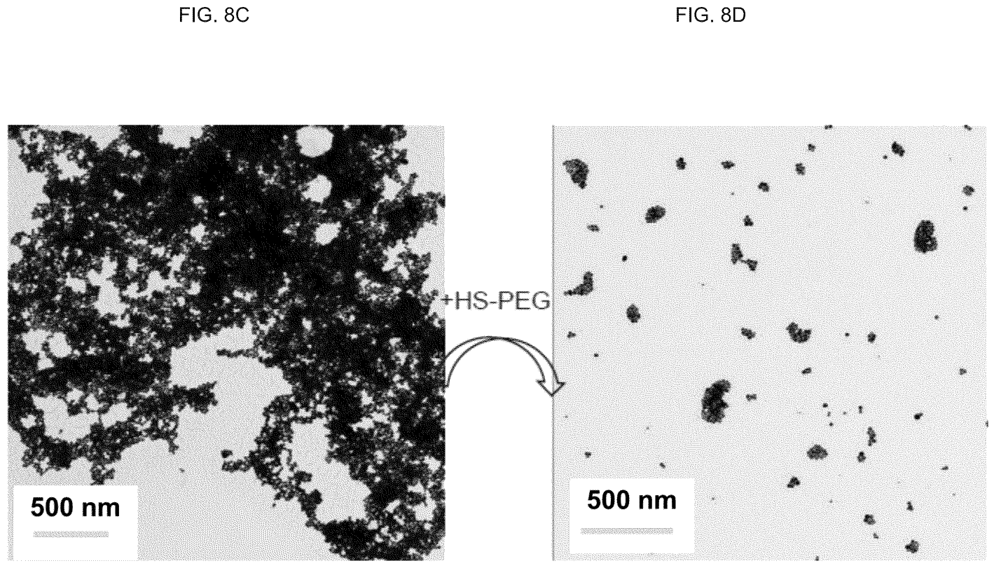

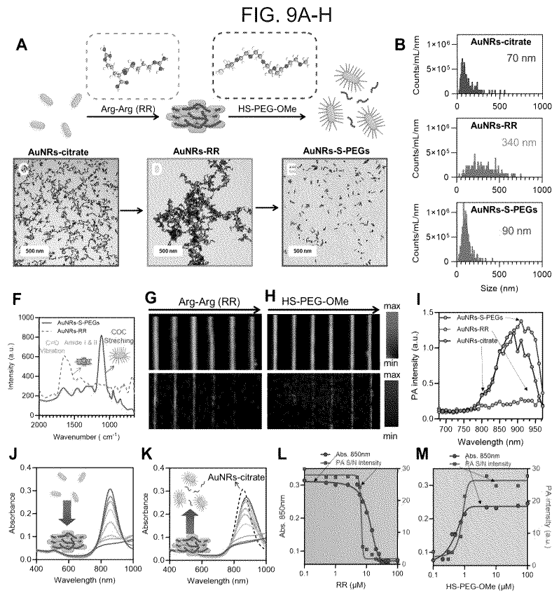

Peptides containing the motif Arg-Arg-Lys (RRK) were thus conjugated to a thiolated PEG capped with a carboxyl group (HS-PEG₁₂-COOH, Mw=634 Da) via EDC/NHS cross-linking reaction. Three peptides based on the motif RRK were investigated and the number of RRK repetitions varied from 1 to 3 to increase the mass and charge of the resulting HS-PEG-peptide conjugate (FIG. 6A).

FIG. 6. Trypsin sensing with HS-PEG-peptide conjugates. (A) Scheme of the synthesis of HS-PEG-peptide conjugates via EDC/NHS chemistry including the three main sequences investigated as well as the size and charge of the compounds. (B) Illustration of the sensing mechanism: The trypsin cleavage reduces the size and charge of the HS-PEG-peptide conjugate, which increases their capacity to dissociate the AuNPs assemblies. Dissociation of Arg-Arg-AuNPs assemblies with either (C) HS-PEG-RRK, (D) HS-PEG-RRKRRK (SEQ ID NO:13), or (E) HS-PEG-RRKRRKRRK (SEQ ID NO:14) intact (black line) or digested by 1 μM of trypsin for 24 h at 37° C. (dashed blue line). (F) Dissociation of Arg-Arg-AuNPs assemblies with HS-PEG-TSG and HS-PEG-C12 either intact or digested by trypsin. (G) Trypsin detection using the three different HS-PEG-peptide conjugates and (H) the corresponding pictures. (I) Picture of dried Arg-Arg-AuNPs assemblies after the addition of 50 μL of HS-PEG-RRKRRK (SEQ ID NO:13) incubated or not with 1 μM of trypsin. (J) Comparison between the detection of 1 μM of trypsin spiked either in PBS or in urine as well as in PBS with 80 μM of AEBSF inhibitor.

From concentrations of 10 μM, HS-PEG₁₂-COOH is capable to dissociate 100% of the assemblies (FIG. 21). However, the conjugation of the peptides to the HS-PEG₁₂-COOH was expected to decrease its capacity to dissociate the assemblies as it increases its size and charge. In this context, the addition of the HS-PEG-peptide conjugates to AuNPs assemblies could not generate a colorimetric signal. However, in the presence of trypsin, the proteolytic cleavage could progressively remove the amino acids residues from the conjugate, thus reducing its size and charge that would restore the dissociation capacity. A colorimetric signal, proportional to the trypsin activity, could thus be observed (FIG. 6B).

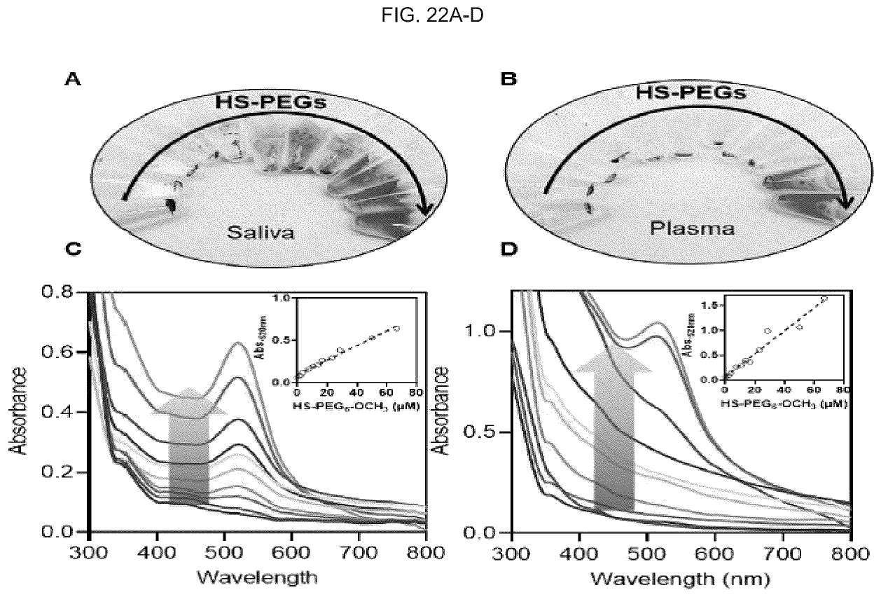

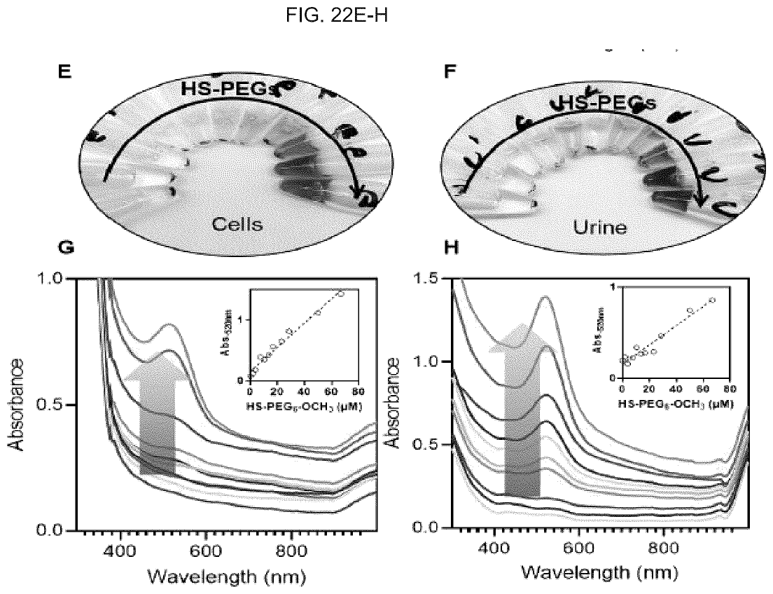

The HS-PEG-peptide conjugates were synthesized and titrated to Arg-Arg-AuNP assemblies in order to evaluate the quenching of their dissociation capacity. EDC/NHS cross-linking between HS-PEG-COOH and RRKRRK (SEQ ID NO:13) or RRKRRKRRK (SEQ ID NO:14) led to a total quenching of the dissociation capacity of HS-PEG₁₂-COOH even at high concentrations (greater than (>) 100 μM) (FIGS. 6D and 6F) while the conjugation of RRK only reduced it slightly (FIG. 6E). In the absence of EDC/NHS, no cross-linking could occur and the dissociation capacity of HS-PEG₁₂-COOH was not affected (FIG. 22).

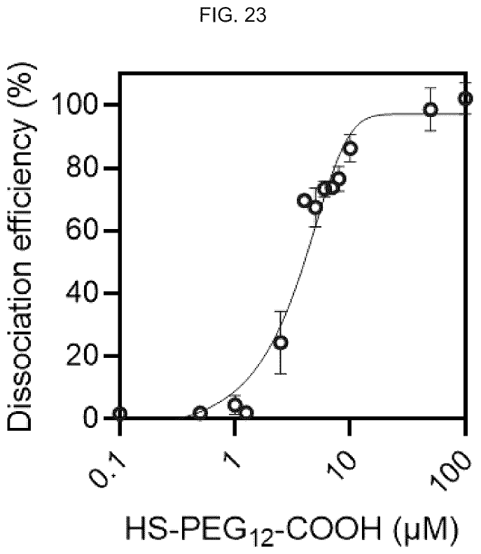

Subsequently, the three conjugates were digested with trypsin (10 μM, 37° C., 24 h) and titrated into the AuNPs assemblies again. Interestingly, the dissociation capacity of HS-PEG-RRKRRK (SEQ ID NO:13) and HS-PEG-RRKRRKRRK (SEQ ID NO:14) was restored; HS-PEG-RRK was also enhanced because cleavage by trypsin reduced the size and the positive charge of the conjugate (FIGS. 6C, 6D and 6E). A minimum of two repetitions of the motif RRK is thus necessary to ensure a total quenching of the dissociation capacity of the HS-PEG₁₂-COOH. This can later be completely restored with the proteolytic cleavage. Only one repetition of RRK leads to a very narrow window of detection between the intact and digested HS-PEG-peptide. In addition to conferring a higher positive charge, the RRKRRK (SEQ ID NO:13) and RRKRRKRRK (SEQ ID NO:14) peptides could conjugate multiple HS-PEGs per peptide due to the presence of lysine residues that in turn lead to an even bigger construct. Matrix-assisted laser desorption/ionization (MALDI) was used to characterize the HS-PEG-RRKRRK (SEQ ID NO:13) conjugate (FIG. 23) and a mixture of single and multiple conjugations of HS-PEG per peptide were observed.

Control experiments were then conducted with alternative non-trypsin cleavable molecules: the peptide TSG and the bis-amine C₁₂. The resulting conjugates (HS-PEG-TSG and HS-PEG-C₁₂) were then titrated to Arg-Arg-AuNPs assemblies before and after digestion with trypsin, similarly to what was described previously. The dissociation capacity of HS-PEG-TSG was similar to the one of HS-PEG₁₂-COOH because TSG is a short peptide and lacks positive charge. It is not impacted by the digestion by trypsin. On the other hand, the conjugation of C₁₂ led to total quenching of the dissociation capacity that could not be restored with trypsin digestion. This is because C₁₂, like RRKRRK (SEQ ID NO:13) and RRKRRKRRK (SEQ ID NO:14), contains more than one free amine and can thus be conjugated to multiple HS-PEG molecules. This makes the conjugate too bulky to dissociate the AuNPs. However, unlike RRKRRK (SEQ ID NO:13) and RRKRRKRRK (SEQ ID NO:14), C₁₂ does not have any cleavable site for trypsin and thus the trypsin digestion has no effect on the dissociation capacity (FIG. 6F).

### Conclusion

In summary, the dissociation of AuNPs assemblies with HS-PEGs molecules was studied and exploited to build matrix-insensitive sensors. Robust assemblies of AuNPs-citrate were formed using a di-arginine additive (Arg-Arg). The efficient electrostatic interactions between the citrate and the arginine led to compact assemblies of the particles, thus provoking a strong modification of their optical properties. However, the presence of peptides protected the AuNPs from degradation. Surprisingly, the addition of HS-PEGs could dissociate the assemblies with a total recovery of the initial optical properties. The mechanism was fully characterized by TEM, MANTA, UV-Vis, and FTIR spectroscopies. The HS-PEGs can progressively graft onto the AuNPs surface and remove the citrate/arginine layers. As the hydrophilic PEG layer surrounds the AuNPs, the particles progressively detach from the bulky assemblies and become water-dispersible. Importantly, only a minimum amount of HS-PEGs is needed to cover all the gold surfaces (approximately 4 HS-PEG₆-OCH₃/nm²). We have thus shown that the dissociation capacity of HS-PEGs is modulated by their size and charge. HS-PEG-OCH₃ with a molecular weight of 1000 Da or less could dissociate 80% or more. Remarkably, the dissociation of the assemblies was matrix-insensitive and produced an unambiguous color change in plasma, saliva, urine, bile, cell lysates, or even sea water. Moreover, we found that the generation of the colorimetric signal could be improved by using dried film of AuNPs assemblies. The presence of HS-PEGs leads to the detachment of AuNPs from the surface as it solubilizes them. The color of the suspension becomes then red and its intensity is proportional to the amount of HS-PEGs. In the absence of this later, the color of the solution is clear. This strategy allows a unambiguous distinction with the naked eyes between samples that have or not HS-PEGs. We thus designed a sensing strategy based on HS-PEG-peptide probes and AuNPs assemblies as signal read out for protease sensing in complex media. Trypsin was chosen as model protease and peptide containing repetition of the motif RRK were conjugated to HS-PEGs. The optimized conjugate, HS-PEG-RRKRRK (SEQ ID NO:13), allowed the visual detection of trypsin with a picomolar limit of detection. Detection could be performed simply in pooled urine or saliva spiked with trypsin. This is the first time that HS-PEGs molecules have been used to dissociate AuNPs assemblies or to solubilize dried AuNPs and combined with peptides for protease detection. This innovative approach can benefit protease detection across various complex environments. The approach can be adapted to any protease as long as a peptide substrate can be conjugated to HS-PEGs and the dissociation capacity of the resulting conjugate can only be restored by the proteolytic activity.

### Materials and Methods

G Bis(p-sulfonatophenyl)phenylphosphine dihydrate dipotassium salt (BSPP, 97%), gold(III) chloride trihydrate (HAuCl₄·3H₂O, greater than (>) 99.9%), sodium citrate tribasic dihydrate (>99%), trypsin, 4-mercaptobenzoic acid (MBA, 99%), 3-mercaptopropionic acid (MPA, >99%), N-hydroxysuccinimide (NHS, 98%), 1-12-diaminododecane (C12, 98%), 0-(2-mercaptoethyl)-O′-methyl-hexa(ethylene glycol) (HS-PEG₆-OCH₃, 98%), DL-dithiothreitol (DTT, >99%) and pooled human plasma were purchased from Sigma Aldrich (St. Louis, MO). HS-PEG-OCH₃ Mw=20,000, 10,000, 5,000 and 2,000 g·mol⁻¹ and HS-PEG-NH₂ Mw=10,000 g·mol⁻¹ were purchased from Laysan Bio, Inc. (Arab, AL). HS-PEG-OCH₃ Mw=1,000 g·mol⁻¹ and LA-PEG-OCH³ Mw=1000 g·mol⁻¹ was purchased from Biopharma PEG (Watertown, MA). HS-PEG-OCH₃ Mw=224 g·mol⁻¹, HS-PEG-COOH Mw=600 g·mol⁻¹, Alk-PEG-OH Mw=232.27 g·mol⁻¹, and OH-PEG-OCH₃ Mw=296.36 g·mol⁻¹ were purchased from PUREPEG™ (San Diego, CA). AuNPs-citrate (40 nm) were purchased from NANOCOMPOSIX™ (San Diego, CA). The (1-ethyl-3-(3-dimethylaminopropyl)carbodiimide hydrochloride) (EDC) was purchased from Thermofisher (Waltham, MA). Gibco Phosphate Buffer Saline (PBS) pH 7.4 was purchased from Fisher Scientific (Pittsburgh, PA). The 4-(2-Aminoethyl)-benzenesulfonyl fluoride, HCl (AEBSF) was purchased from Research Product International (Mount Prospect, IL). Peptides Arg-Arg-Lys (RRK), Arg-Arg-Lys-Arg-Arg-Lys (RRKRRK) (SEQ ID NO:13) and Arg-Arg-Lys-Arg-Arg-Lys-Arg-Arg-Lys (RRKRRKRRK) (SEQ ID NO:14) were purchased from GENSCRIPT PROBIO™ (Piscataway, NJ). Sodium chloride (NaCl), potassium chloride (KCl), ferrous chloride (FeCl₂), nitric acid (HNO₃), copper (II) chloride (CuCl₂), and magnesium chloride (MgCl₂) were purchased from Fisher Scientific International, Inc. (Hampton, NH). Pooled human saliva was purchased from Lee Biosolutions, Inc. (Maryland Heights, MO). Pooled human urine filtered was purchased from Innovative Research (Novi, MI). Human bile was purchased from Zen Bio (Durham, NC). Sea water was collected in Pacific Beach (San Diego, CA). Fmoc-protected L/D-amino acids, hexafluorophosphate benzotriazole tetramethyl uronium (HBTU), and Fmoc-Rink amide MBHA resin (0.67 mmol/g, 100-150 mesh) were purchased from AappTec, LLC (Louisville, KY). Organic solvents including N,N-dimethylformamide (DMF, sequencing grade), acetonitrile (ACN, HPLC grade), ethyl ether (certified ACS), methylene chloride (DCM, certified ACS), and dimethyl sulfoxide (DMSO, certified ACS) were from Fisher Scientific International, Inc. (Hampton, NH). Ultrapure water (18 MΩ·cm) was obtained from a Milli-Q Academic water purification system (Millipore Corp., Billerica, MA). Amicon® ultra-15 centrifugal filter units (M.W. cutoff=100 kDa) and automation compatible syringe filters (PTFE, 0.45 mm) were from MilliporeSigma (St. Louis, MO). Glassware and stir bars were cleaned with aqua regia (HCl:HNO3=3:1 by volume) and boiling water before use. Beside aqua regia that has to be handled carefully, no unexpected or unusually high safety hazards were encountered.

### AuNPs Synthesis

Citrate-stabilized AuNPs (approximately 20 nm) were prepared using the Turkevich method (see for example, Kimling et al J. Phys. Chem. B 2006, 110, 32, 15700-15707) by rapidly injecting an aqueous solution of sodium citrate tribasic dihydrate (150 mg, 5 mL) into an aqueous solution of HAuCl₄·3H₂O (45 mg, 300 mL) under boiling conditions and vigorous stirring. The reaction mixture was left boiling while stirring for another 15 min and then cooled down to room temperature. The deep red dispersion was then purified by applying one round of centrifugation at 18,000 g for 30 min, and the pink supernatant was discarded. The resulting pellet of AuNPs-citrate was redispersed in deionized water by sonication and stored at ambient conditions.

AuNPs Assembly and Dissociation Assembly. Typically, 1 mL of AuNPs-citrate at OD=1.5 was mixed with 50 μL of Arg-Arg (100 μM) to provoke the AuNPs assembly. The color of the suspensions rapidly changed from red to blue. The assembled AuNPs (Arg-Arg-AuNPs) were stable over time when stored at 4° C. and could be used for dissociation even month after their assembly.

Dissociation. Stock solutions of the different HS-PEGs with concentrations ranging from 10 μM to 1 mM were prepared. Specific volumes of each solution were added to a 96-well plate in order to reach the desired final HS-PEGs concentration and then 100 μL of Arg-Arg-AuNPs were added. For complex matrices experiments, the Arg-Arg-AuNPs were first concentrated 10 times by centrifugation and then dispersed in complex media (sea water, pooled human saliva, plasma, urine, bile or HEK cell lysates) that represented thus 90% of the total volume except for bile, which was only 20%. Quickly after the addition of AuNPs, the dissociation of the assembly was monitored and the ratio of the absorbances at 520 nm and 700 nm was recorded over time. The percentage of dissociation is described as follows:

$$\% of dissociation = \frac{{( \frac{Abs 520 nm}{Abs 700 n m} )}_{AuNPs - S - PEGs} - {( \frac{Abs 520 nm}{Abs 700 nm} )}_{Arg - Arg - AuNPs}}{{( \frac{Abs 520 nm}{Abs 700 nm} )}_{AuNPs - citrate} - {( \frac{Abs 520 nm}{Abs 700 nm} )}_{Arg - Arg - AuNPs}}$$

### Where

$${( \frac{Abs 520 nm}{Abs 700 n m} )}_{AuNPs - S - PEGs}$$

is the ratio of the absorbance after dissociation with HS-PEGs,

$${( \frac{Abs 520 nm}{Abs 700 nm} )}_{AuNPs - citrate}$$

is the ratio of the absorbance of the initial citrate-capped AuNPs, and

$${( \frac{Abs 520 nm}{Abs 700 nm} )}_{Arg - Arg - AuNPs}$$

is the ratio of the arginine-induced assembly of AuNPs.

### Drying and Solubilization of the AuNPs Assemblies

First, 1 mL of AuNPs-citrate was concentrated 5 times by centrifugation (18,000 g during 18 minutes). Then, the assembly was provoked by adding 25 μL of Arg-Arg (100 μM) to the resulting 200 μL of concentrated AuNPs-citrate. Finally, 10 μL of the concentrated Arg-Arg-AuNPs were added to an Eppendorf and dried at 40° C. overnight. The solubilization of the dried assemblies with HS-PEGs was performed similarly to the dissociation of the assemblies. It is worth noting that shaking vigorously the Eppendorf could be required.

### Peptide Synthesis

Peptides Arg-Arg (RR), Arg-Arg-Arg-Arg-Arg (RRRRR) (SEQ ID NO:15), Arg-Gly-Gly-Gly-Arg (RGGGR) (SEQ ID NO:16) and Tyr-Ser-Gly (TSG) were synthesized using an automated Eclipse™ peptide synthesizer (AAPPTEC™, Louisville, KY) through standard solid phase Fmoc synthesis on Rink-amide resin. Peptides were lyophilized in a FREEZONE PLUS 2.5™ freeze dry system (Labconco Corp., Kansas, MO).

Peptide purification used a Shimadzu LC-40 HPLC system equipped with a LC-40D solvent delivery module, photodiode array detector SPD-M40™, and degassing unit DGU-403. The crude sample was dissolved in an acetonitrile/H₂O mixture (1:1, v/v) with an injection volume of 2 mL. This was applied on a ZORBAX 300 BS™, C18 column (5 μm, 9.4×250 mm) from Agilent (Santa Clara, CA) and eluted at a flow rate of 1.5 mL/min over a 40 min linear gradient from 10% to 95% of acetonitrile in water (with 0.05% TFA, HPLC grade). Preparative injections were monitored at an absorbance of 190, 220, and 254 nm. Fractions containing the pure peptide as confirmed by electrospray ionization mass spectroscopy were lyophilized and aliquoted (see below). All peptides were purified by HPLC to reach a purity of at least 90%.

Peptide synthesis and cleavage was confirmed using Electrospray ionization mass spectrometry (ESI-MS, positive ion mode) via the Micromass Quattro Ultima mass spectrometer in the Molecular MS Facility (MMSF). ESI-MS samples were prepared in a MeOH/H₂O mixture (1:1, v/v).

### HS-PEG-Peptide Conjugate Synthesis

Typically, 1 mL of HS-PEG₁₂-COOH (HS—(CH₂CH₂O)₁₂—CH₂CH₂COOH, 634 Da, 1 mM) dissolved in MES buffer (10 mM, pH=5.5) was activated via the addition of 100 μL of EDC (50 mM, MES) and 40 μL of NHS (500 mM, MES). The reaction was stirred at room temperature for one hour. After one hour, 1 mL of peptide (or NH₂-molecule) (1 mM) dissolved in PBS (100 mM, pH=7.4) was added to the activated HS-PEG₁₂-COOH. The reaction was stirred for 4 hours at room temperature and then stored at 4° C.

### Trypsin Incubation

Typically, 5 μL of the PEG-peptide conjugate (0.44 mM) were added to 5 μL of PBS 1×. Subsequently, 2 μL of trypsin of various concentrations were added and the resulting solution was incubated at 37.5° C. for 2 hours. At the end of the incubation, 100 μL of aggregated AuNPs (Arg-Arg-AuNPs) were added, and a color change proportional to the trypsin concentration was observed. UV-Vis spectra were recorded 30 minutes later.

### Trypsin Inhibition Study

Aqueous AEBSF inhibitor solution (20 mM) was prepared, and a specific volume was mixed with 1 μM of trypsin in order to reach the desired final AEBSF concentration. The resulting mixture was stirred gently at room temperature for 30 minutes. Subsequently, the specific volume of HS-PEG-RRKRRK (SEQ ID NO:13) was added to reach a final concentration of 200 μM and the solution was incubated at 37.5° C. for 2 hours. Finally, 100 μL of Arg-Arg-AuNPs (20 nm) were added, and the UV-Vis spectrum was recorded 10 minutes later.

### UV-Vis Spectroscopy

The optical absorption measurements were collected using a hybrid multi-mode microplate reader (Synergy™ H1 model, BioTek Instruments, Inc.) in a 96-well plate. The dissociation of assembly was characterized by measuring the ratio of the absorbance at 520 nm or 530 nm and 700 nm or 820 nm for 20 nm NPs or 40 nm NPs, respectively.

### ATR-FTIR Spectroscopy

ATR-FTIR spectra were recorded with a Nicolet™ iS50 FTIR Spectrometer with a DLaTGS detector by natural drying of 1 μL of AuNPs suspensions. Typically, 1 mL of AuNPs (2 nM) was first cleaned from unbound molecules via four cycles of centrifugation (18,000 g during 18 minutes) and resuspended in 40 μL of pure water. The final concentration was then approximately 50 nM.

### Transmission Electron Microscopy (TEM)

Transmission electron microscopy (TEM) images of the Au colloids were acquired using a JEOL 1200 EX II operating at 80 kV. The TEM grids were prepared by natural drying of 2 μL of 2 nM AuNPs.

### Multispectral Advanced Nanoparticles Tracking Analysis (MANTA)

The multispectral advanced nanoparticle tracking analysis (MANTA) is a technique that builds images from the particles' light scattering via three lasers of different wavelengths (for example, blue, green, and red); the wavelength of scattering depends on nanoparticle size. Thus, small particles (less than, or <, 100 nm) appear blue while larger particles appear greener or redder. MANTA uses these images to count the nanoparticles and calculate their size based on Brownian motion. MANTA was performed with the VIEWSIZER 3000™ (Horiba Scientific, USA). The temperature was set to 25° C. during the measurement. Automated noise analysis determines the optimal wavelength for representing each nanoparticle. Here, 8-bit composite videos were generated, and 10 videos were used per analysis (300 frames for seconds). A quartz cuvette with minimum volume of 1 mL was used for the measurement and the AuNPs concentrations was set up at 0.04 nM.

### Cell Culture

A human embryonic kidney cell line (HEK 293T) was used for this work. The cells were cultured in Dulbecco's modified Eagle's medium (DMEM) with 10% fetal bovine serum (FBS) and 1% penicillin-streptomycin. The cells were incubated at 37° C., 5% CO₂, and the media was replaced every two days. Cells were passaged at 75-80% confluency using Trypsin-EDTA (0.25%); 1,000,000 HEK 293T cell samples were harvested by detaching the cells with Trypsin-EDTA (0.25%), centrifugation at 700 relative centrifugal force (RCF) for 5 minutes, and resuspending in PBS for further experiments. Cell lysate was prepared by mechanical disruption via freeze-thaw cycles.

### Example 2: Peptide-Based Reversible Aggregation and Biosensing

This example demonstrates that methods and compositions as provided herein using the exemplary embodiments as described herein are effective in detecting a protein in any biological fluid.

Colorimetric biosensors based on gold nanoparticle (AuNP) aggregation are often challenged by matrix interference in biofluids, poor specificity, and limited utility with clinical samples. Here, we propose a peptide-driven nanoscale disassembly approach, where AuNP aggregates induced by electrostatic attractions are dissociated in response to proteolytic cleavage. Initially, citrate-coated AuNPs were assembled via a short cationic peptide (RRK) and characterized by experiments and simulations. The dissociation peptides were then used to reversibly dissociate the AuNP aggregates as a function of target protease detection, i.e., main protease (M^{pro}), a biomarker for SARS-CoV-2. The dissociation propensity depends on peptide length, hydrophilicity, charge, and ligand architecture. Finally, this exemplary dissociation strategy provides a rapid and distinct optical signal through M^{pro} cleavage with a detection limit of 12.3 nM in saliva. This exemplary dissociation peptide effectively dissociates plasmonic assemblies in diverse matrices including 100% human saliva, urine, plasma, and seawater, as well as other types of plasmonic nanoparticles such as silver. This exemplary peptide-enabled dissociation platform provides a simple, matrix-insensitive, and versatile method for protease sensing.

Embodiments described in Example 1 include colorimetric assays based on dissociation. Particle aggregation was first triggered by a short cationic peptide containing arginine and lysine that induced plasmonic coupling of anionic AuNPs coated with citrate. Most importantly, this aggregation was reversible upon addition of steric stabilizers such as thiol-PEG molecules (HS-PEGs). We then made a PEG-peptide conjugate containing a cleavage sequence specific to trypsin. The first value of this approach was a remarkable insensitivity to the matrix-dispersion could even be done in seawater and bile. Second, the aggregated AuNPs were stable for months and could even be dried to completeness but could still be redissolved and used to detect proteases.

In the work described in this Example, we used computational methods to better understand the mechanism of this reversible aggregation. We hypothesized that the short cationic peptide has a steric bulk that maintains some separation distance between the AuNPs thus preventing runaway attractive VdW attraction. We used an all-peptide strategy that is simpler and requires no PEG-peptide couplings. We demonstrated that charge, hydrophilicity, peptide length, and ligand architecture can impact on the dissociation efficiency. Finally, we constructed a practical sensor that is made of the optimized dissociation domain with a biomolecular recognition element of SARS-CoV-2 main protease i.e., M^{pro}.^{17,18} After protease cleavage, released peptides successfully provided a rapid color readout of M^{pro} with a limit of detection (LoD) of 12.3 nM in saliva. Furthermore, this exemplary dissociation peptide can dissociate AuNP aggregates in various matrixes including 100% human urine, plasma, and seawater, and can be applied to other types of plasmonic nanoparticles (for example, silver).

### Results

### Short Cationic Peptides for Reversible Aggregation.

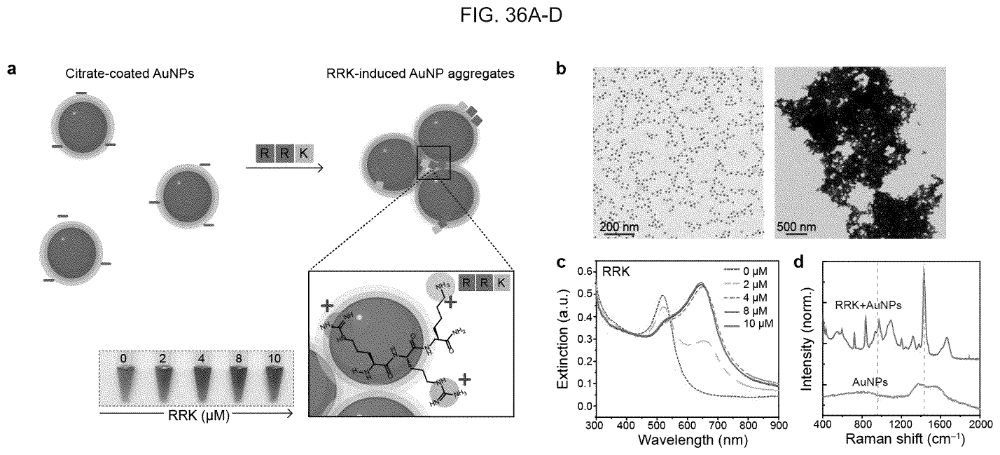

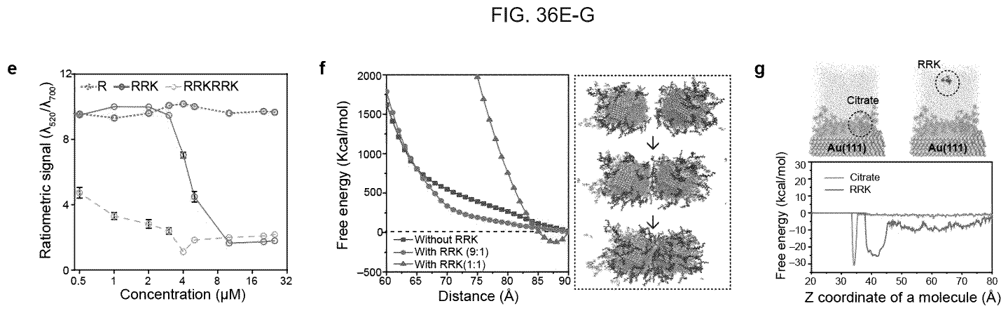

To induce reversible aggregation of the AuNPs, we used positively charged Arg and Lys-based peptide residues (i.e., RRK). The RRK peptide could induce plasmonic coupling by electrostatic attractions between negatively charged citrate molecules and guanidine and amine groups in RRK (FIG. 36a).^{8,19} The size of AuNPs increased upon addition of RRK peptides, as confirmed by transmission electron microscopy (TEM) and dynamic light scattering (DLS) (FIG. 36b and FIG. 42). UV-vis spectroscopy showed that plasmonic resonance peak (520 nm) of AuNPs red-shifted to 648 nm; by eye the sample changed color from red to blue (FIG. 1c). Raman spectroscopy observed the appearance of C—C stretching at 984 cm⁻¹ and C—N stretching at 1443 cm⁻¹ from Arg residues in RRK (FIG. 36d).²⁰ Lastly, R, RRK, and RRKRRK (SEQ ID NO:13) peptides (from 0.5 to 30 μM) were used to induce AuNP aggregates to study the impact of charge number on particle aggregation. The results showed that RRKRRK (SEQ ID NO:13) and RRK peptides effectively induced particle aggregation whereas AuNPs did not aggregate with just one R even at 8-fold higher concentration than RRK (FIG. 36e). A higher positive charge number can decrease the critical coagulation concentration (CCC).⁷ RRK peptide was chosen in this study because particle aggregation induced by a strong cationic peptide (i.e., RRKRRK) (SEQ ID NO:13) can result in irreversible aggregation.

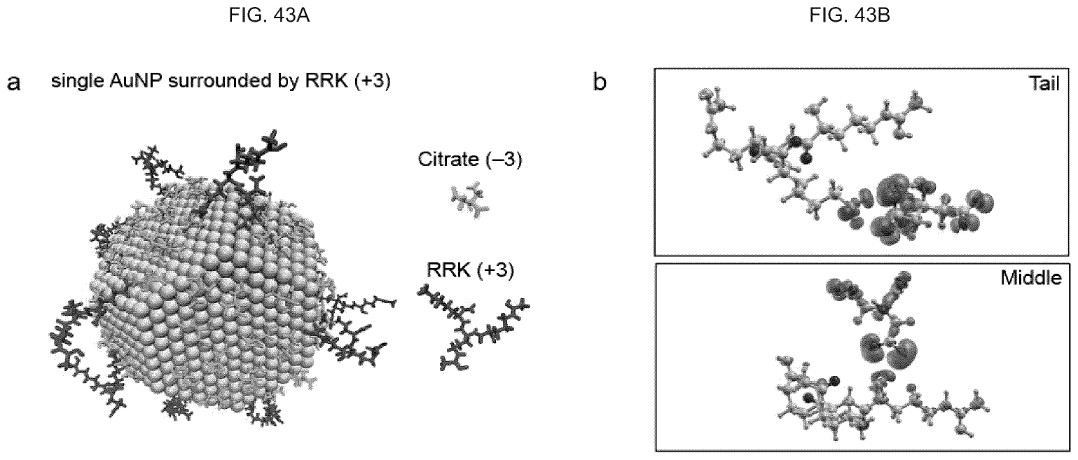

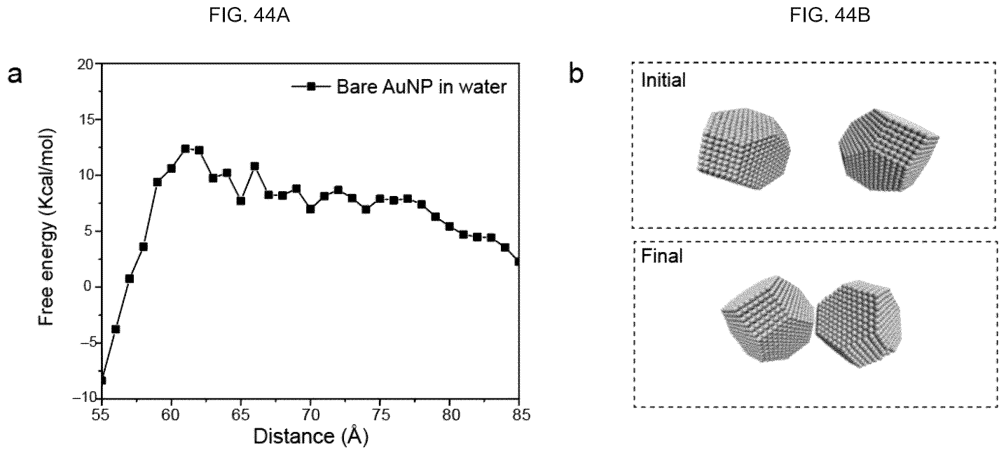

To further study RRK-based plasmonic coupling, we adopted both quantum mechanics (QM) computation and molecular dynamics (MD) simulation. MD simulation revealed that the surface environment of AuNP changed as a function of RRK molecules interacting with citrate-coated AuNPs (FIG. 43). Furthermore, we computed free energies before and after adding RRK peptides into the citrate coated AuNPs via steered molecular dynamics (SMD) simulation. Initially the citrate-coated AuNPs were favorably dispersed due to strong electrostatic repulsions. After adding enough RRK peptides, a free energy minimum point at 87 Å was observed, which suggests a separation distance where nanoparticles were reversibly plasmonically coupled (FIG. 36f and FIG. 44). Lastly, Metadynamic (MTD) simulations evaluated the binding mechanism between Au (111) surface and citrate or RRK molecule, respectively (FIG. 36g). The Z coordinates of citrate and RRK molecule are 34 Å and 39 Å, thus indicating that RRK peptides are favored to bind on the top of a citrate-coated Au (111) surface rather than replacing citrate molecules.

FIG. 36A-G: Short cationic peptides for reversible aggregation. a, Schematic illustration of RRK-based particle aggregation. AuNPs were aggregated by electrostatic attractions between negatively charged citrate on AuNPs and positively charged RRK peptides. The inset photograph shows color change from red to blue as a function of RRK peptide (2-10 μM). b, TEM images of citrate-coated AuNPs (left) and RRK-induced AuNP aggregates (right). c, UV-vis spectrum of RRK-induced AuNP aggregates. The plasmonic resonance peak of AuNPs was red-shifted due to the plasmonic coupling. d, Raman shifts before and after adding RRK peptides into citrate-coated AuNPs. The Raman peak at 1443 cm⁻¹ was attributed to the C—N stretching in Arg residue.²⁰ e, Ratiometric signal (λ₅₂₀/λ₇₀₀) of AuNPs after adding R, RRK, and RRKRRK (SEQ ID NO: 13) peptides with different concentrations 0.5-32 μM, respectively. The error bars represent standard deviation of three independent samples. f, SMD simulations for free energy investigation as a function of AuNP distance at 298K and 1 atm. Energy minimum point was observed after adding RRK peptides. Black, red, and blue lines indicate a citrate to RRK molar ratios of 1:0, 9:1, and 1:1, respectively. The inset images indicate the simulation stages along a trajectory of the citrate-coated AuNPs with RRK (9:1) system. g, Metadynamics (MTD) free energy investigation for a system with 1 RRK on Au(111) surface (right) and a system with 1 citrate on Au(111) surface (left). The Z coordinate value was calculated based on the center mass of RRK and citrate molecule shown in the inset images. The upper surface of Au(111) slab was located at 31 Å. The MTD results observed no surface ligand exchange on Au(111) during the electrostatic interactions between RRK and citrate molecules.

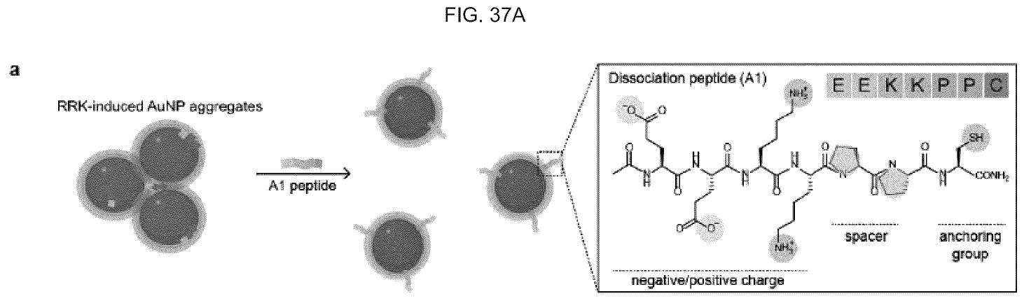

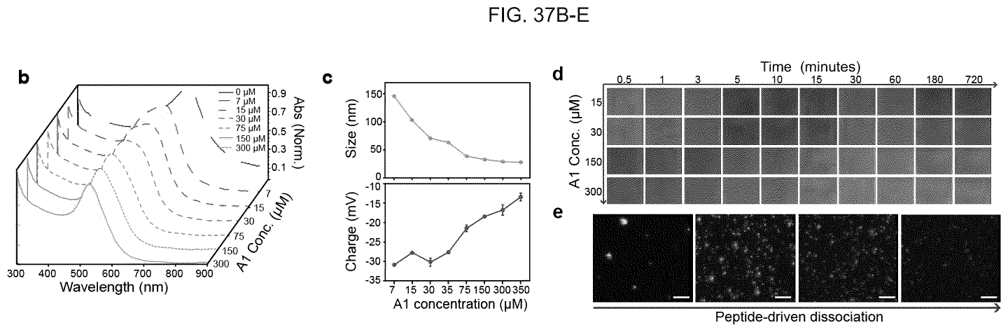

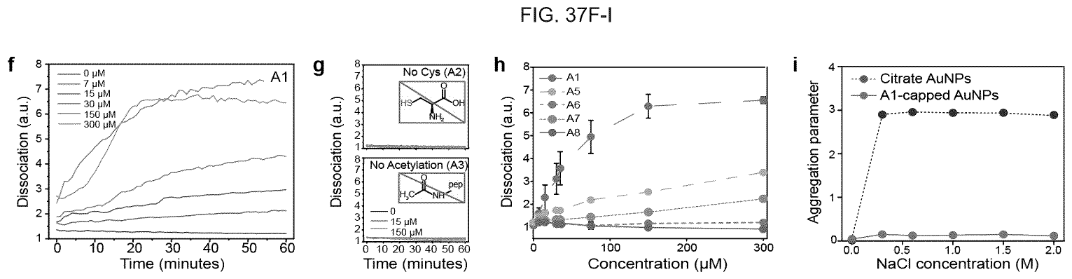

Peptide-enabled dissociation of AuNP aggregates. Peptide-based ligands for AuNPs are of particular interest because of their structural- and chemical-versatilities that can provide high colloidal stability, functionalization, and prevention of protein adsorptions.²¹⁻²⁴ Peptides which could provide electrostatic repulsion, steric distance, and hydrophilicity can attenuate electrostatic attractions induced by RRK, leading to reversible aggregation. We designed these exemplary dissociation peptides (i.e., A1 peptide) to have three major components: charge, spacer, and anchoring groups (FIG. 37a). First, we used Glu (E) and Lys (K) amino acids to provide steric distance and strong hydration layer.²² Negatively charged EE residues provides electrostatic repulsion, and positively charged KK residues in the vicinity of the thiol could increase the grafting kinetics of thiols onto the citrated-coated AuNPs.²⁵ Second, Pro-based linker residues provide a space between charge and anchoring group, further increasing the stability of the AuNPs.²⁶ Lastly, Cys (C) amino acid contains a thiol side chain that binds to the surface of AuNPs via strong Au—S bonds.²⁷

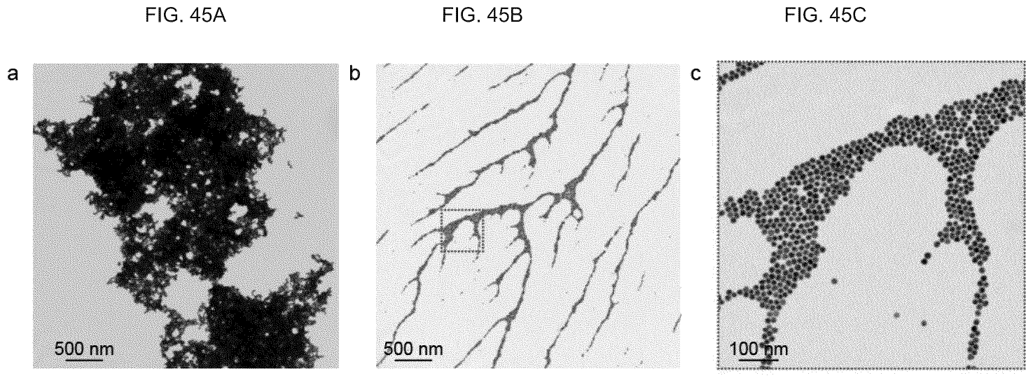

After inducing AuNP aggregates by the RRK peptides, the A1 peptides with different concentrations from 7 to 300 μM were used to dissociate AuNP aggregates. The plasmonic resonance peak of AuNP aggregates blue shifted to 520 nm after particle dissociation. (FIG. 37b). The hydrodynamic diameter of AuNP aggregates (greater than (>) 1 μm) was reduced to 27±0.07 nm, and the surface charge of AuNPs was—13.4±1.03 mV because of surface modification with neutral charged A1 peptides (150 μM) and residual citrates (FIG. 37c). Particle dissociation induced color change from blue to red and a decrease in size as confirmed by time-dependent photographs, multi-laser wavelength nanoparticle tracking analysis (M-NTA), and TEM (FIG. 37d-e and FIG. 45A-C). Ratiometric signal (λ₅₂₀/λ₇₀₀) indicated that A1 peptide (>150 μM) rapidly dissociate AuNP aggregates in 20 minutes (FIG. 37f).

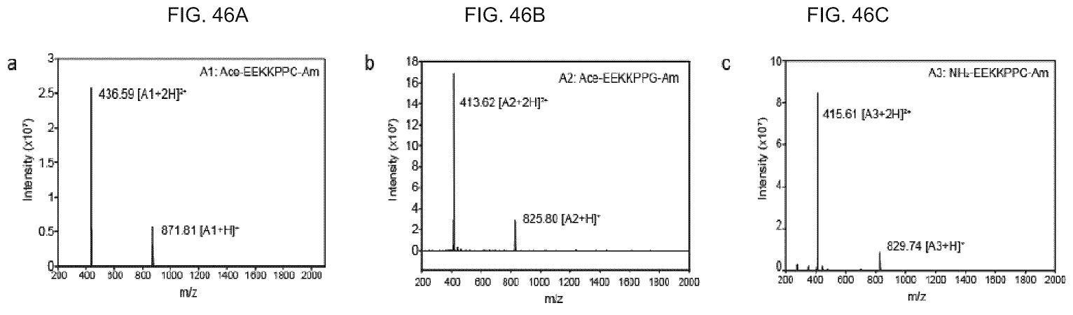

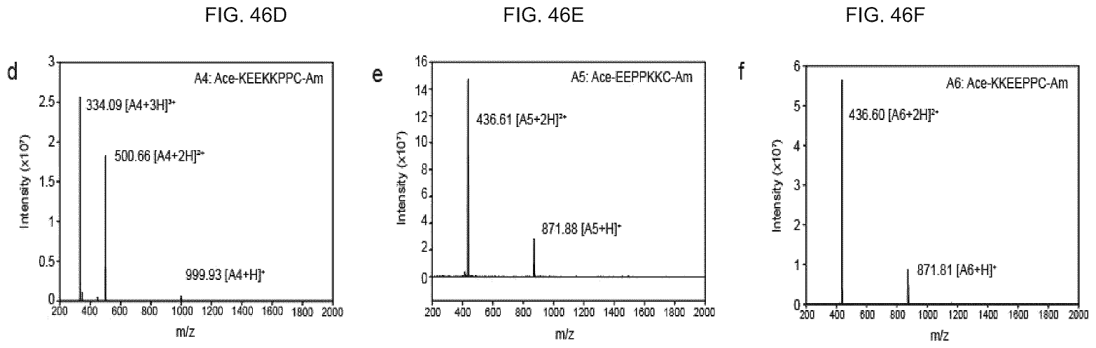

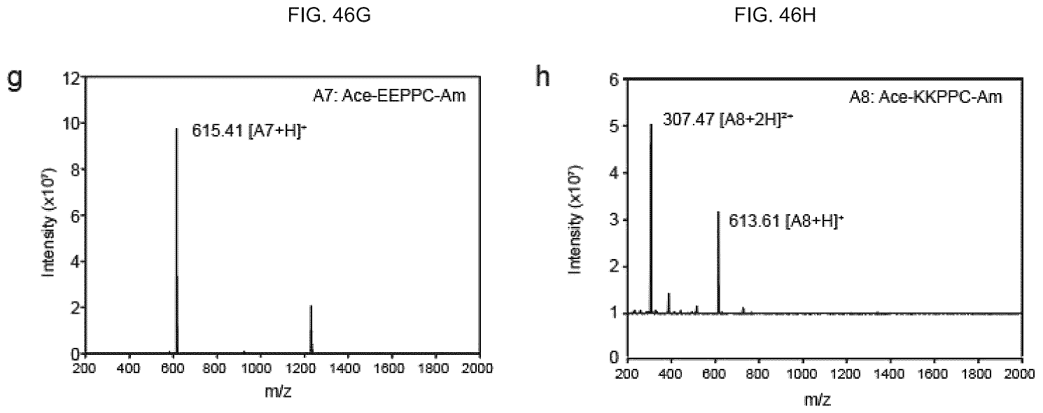

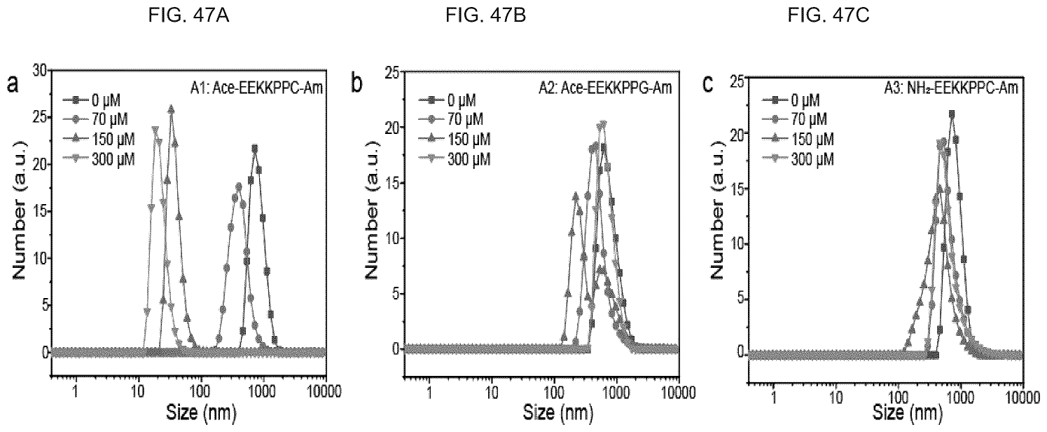

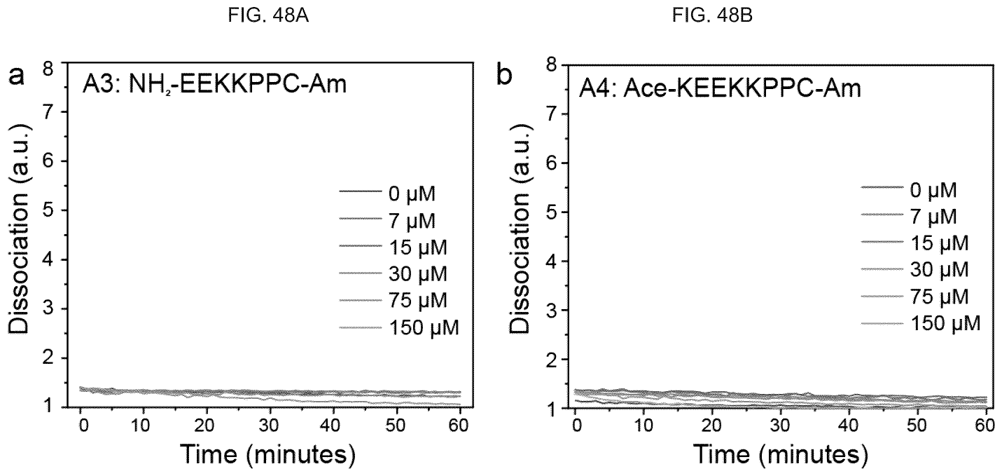

To further study the role of each amino acid in the A1 peptide, we synthesized eight peptide sequences (from A2 to A8) for control experiments (FIG. 46A-H). Table 1 includes peptide sequence, net charge, critical dissociation concentrations (CDC), and design rationale. First, we replaced Cys residue with Gly (i.e., A2) to confirm the role of the anchoring group. Not surprisingly, the A2 peptide failed to dissociate AuNP aggregates due to absence of the Au—S binding (FIG. 37g and FIG. 47A-C). Second, we hypothesized that negatively charged EE residues were required for particle dissociation because they provide electrostatic repulsions for citrate-coated AuNPs. To prove this, the A1 peptide without acetylation (i.e., A3) and one Lys residue (i.e., A4) were synthesized. The A3 and A4 peptides showed no particle dissociations, confirming that positively charged peptide could not dissociate AuNP aggregates likely due to lack of electrostatic repulsions (FIG. 37g and FIG. 48A-B).

We further studied the role of Glu, Lys, and Pro amino acids in A1 peptides (FIG. 37h). For example, Pro-based spacer can provide a rigid and self-assembling monolayer (SAM) that increases the colloidal stability of AuNPs.²⁸ When the spacer was moved to the site between Glu and Lys residues (i.e., A5), the dissociation capacity was 50% lower than A1 peptide. In addition, when the position of Glu was switched with Lys (i.e., A6), particle dissociation was quenched, meaning that the position of positively charged residue is important to maintain the negatively charged electrical double layers. A1 peptide without Lys (i.e., A7) decreased to 30% of the dissociation capacity likely due to the decrease in grafting kinetics of thiol-Au bonds and hydration layers.^{22,25} Lastly, the dissociated AuNPs were incubated in different concentrations (0.5 to 2 μM) of NaCl to examine colloidal stability of the dissociated AuNPs (FIG. 37i). The aggregation parameter defines the variations of absorbance ratio between 520 and 600 nm at the initial and final conditions (see Supporting information).^{6,21} The results showed that the A1 peptide-capped AuNPs showed the higher colloidal stability than citrate-coated AuNPs due to its longer peptide length, rigid SAM, and improved hydrophilicity.²¹

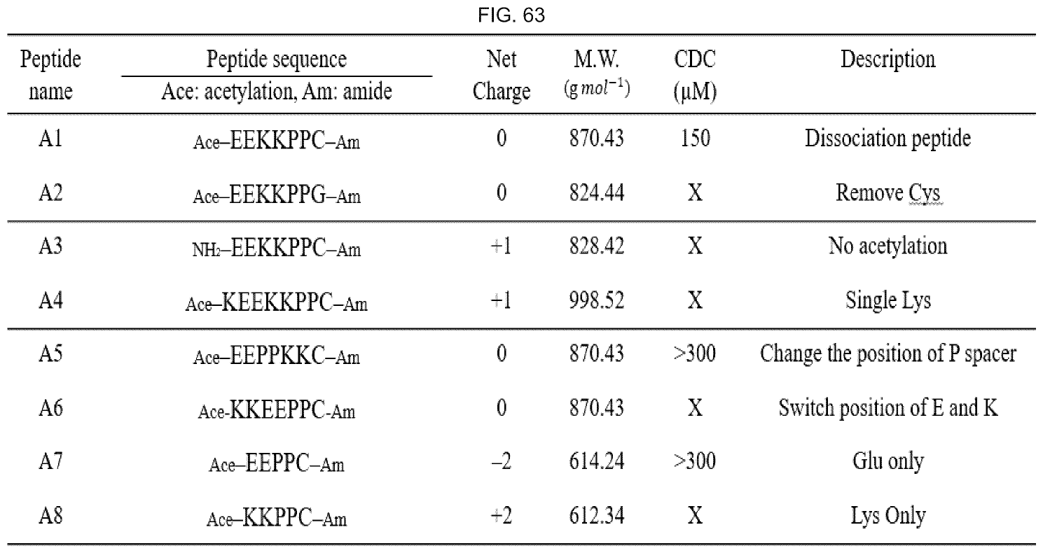

TABLE 2
Al-A8 peptides to study role of structural
components in the dissociation
peptide (see also FIG. 63)

| | Peptide sequence | | | | |
|---|---|---|---|---|---|
| Peptide | Ace: acetylation, | Net | M.W. | CDC | |
| name | Am: amide | Charge | (gmol⁻¹) | (μM) | Description |
| Al | Ace-EEKKPPC-Am | 0 | 870.43 | 150 | Dissociation peptide |
| A2 | Ace-EEKKPPG-Am | 0 | 824.44 | X | Remove Cys |
| A3 | NH₂-EEKKPPC-Am | +1 | 828.42 | X | No acetylation |
| A4 | Ace-KEEKKPPC-Am | +1 | 998.52 | X | Single Lys |
| A5 | Ace-EEPPKKC-Am | 0 | 870.43 | >300 | Change the position of P spacer |
| A6 | Ace-KKEEPPC-Am | 0 | 870.43 | X | Switch position of E and K |
| A7 | Ace-EEPPC-Am | −2 | 614.24 | >300 | Glu only |
| A8 | Ace-KKPPC-Am | +2 | 612.34 | X | Lys Only |

FIG. 37: Peptide-enabled dissociation of AuNP aggregates: a, Schematic illustration of peptide-based particle dissociations. AuNP aggregates induced by RRK peptides were reversibly dissociated by the A1 peptides. The structural component of the A1 peptide contains charge, spacer, and anchoring group. b, UV-vis spectrum shows that the plasmonic resonance peak of AuNP aggregates blue-shifted upon addition of the A1 peptide (7-300 μM). c, Hydrodynamic diameter, and the surface charge after adding the A1 peptide. d, Time-dependent photographs show 150 μM of the A1 peptide requires to dissociate AuNP aggregates. x and y axis indicate time and the A1 concentration, respectively. e, Darkfield images of AuNP aggregates (left) and the dissociated AuNPs (right). The scale bar indicates 10 μm. Blue dots represent actual AuNPs dissociated by the A1 peptide. f, Time-dependent particle dissociation driven by the A1 peptide. The ratiometric signal (λ₅₂₀/λ₇₀₀) was referred as dissociation (y axis). g, Particle dissociation was quenched without Cys (A2), and acetylation (A3). h, Dissociation capacity of the A1, A5, A6, A7 and A8 peptides. i, The dissociated AuNPs by the A1 (red) showed higher colloidal stability than citrate-coated AuNPs (black). Panel c, h and i repeated three independent times and showed similar results.

### Impact of Hydrophilicity and Steric Bulk on Particle Dissociation

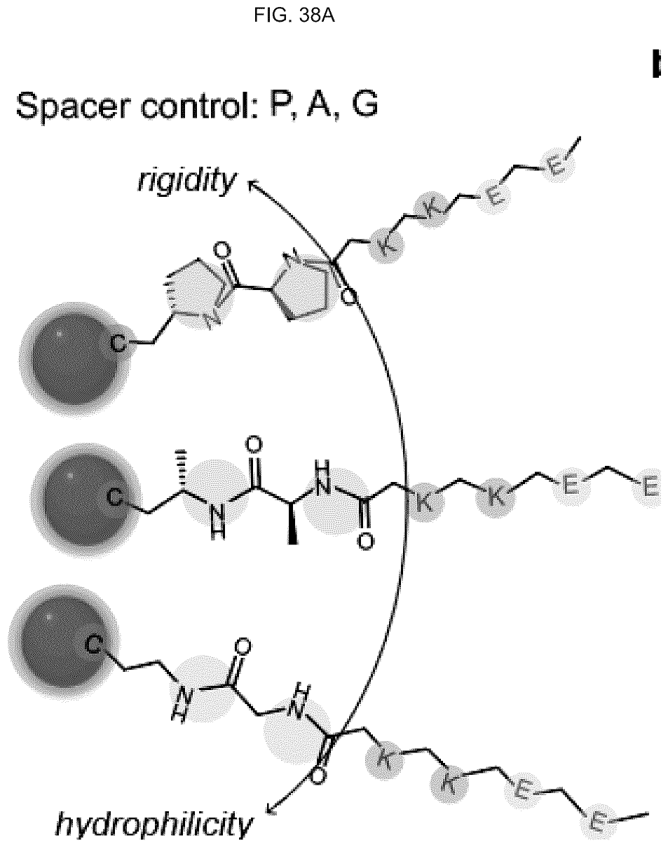

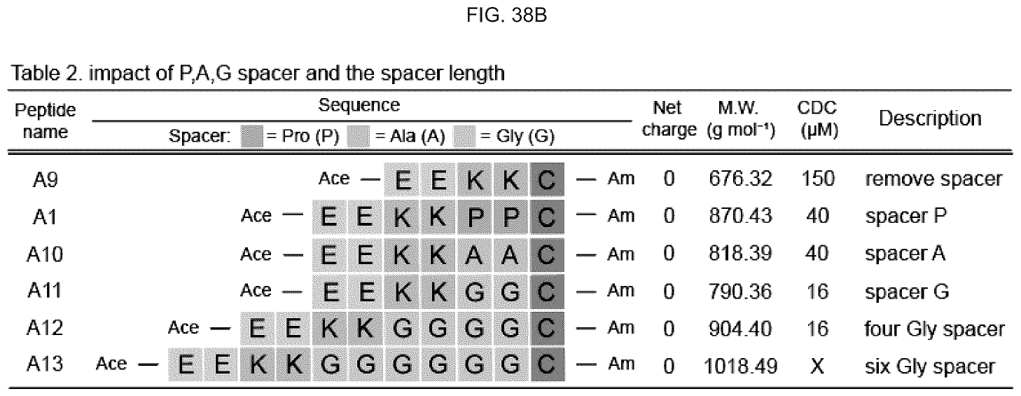

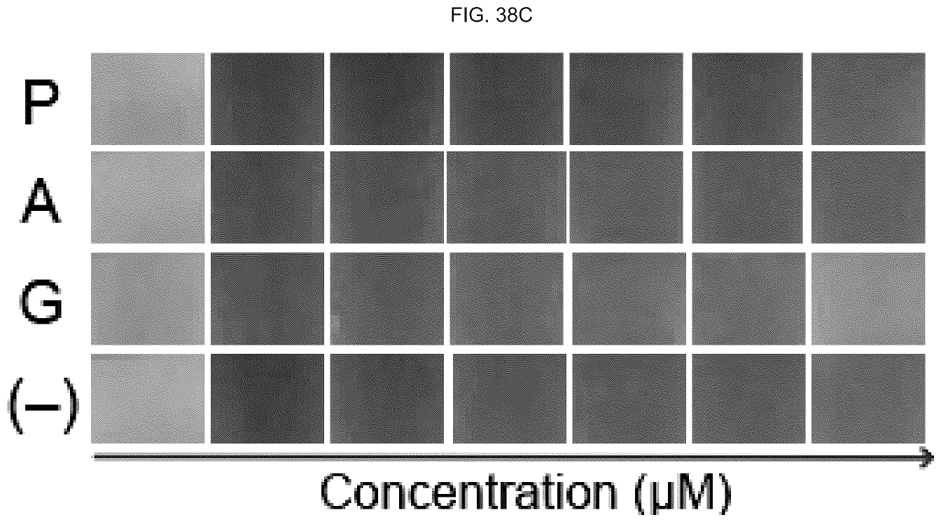

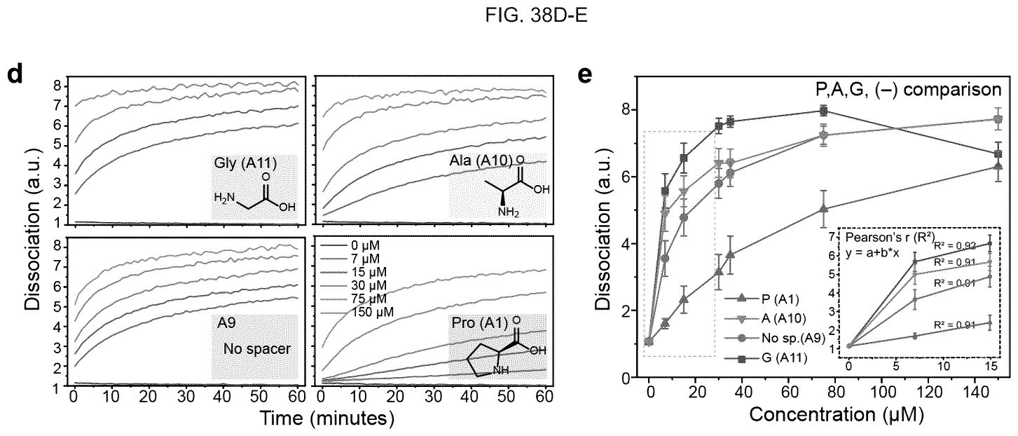

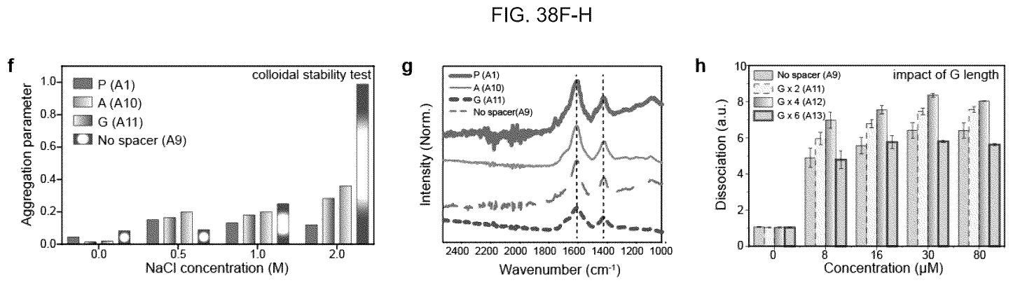

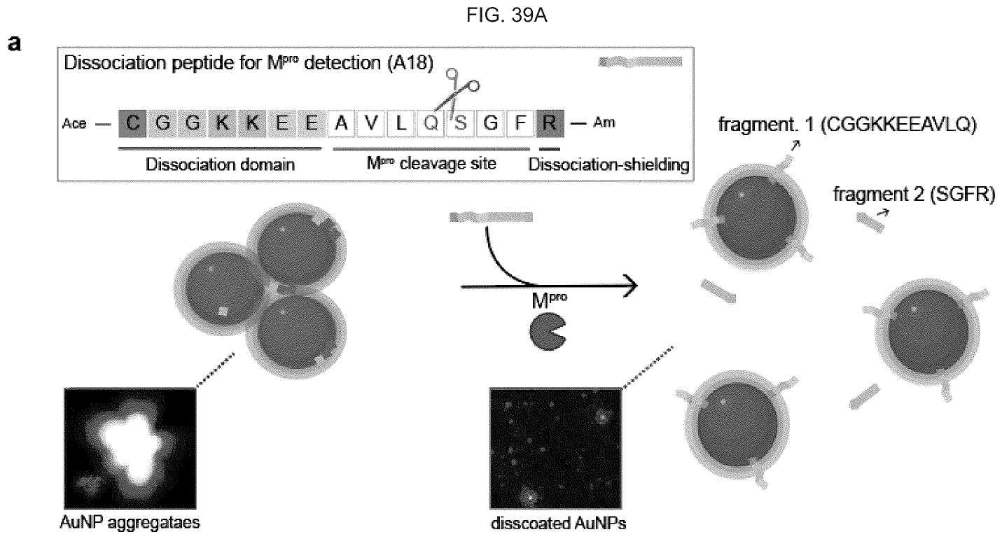

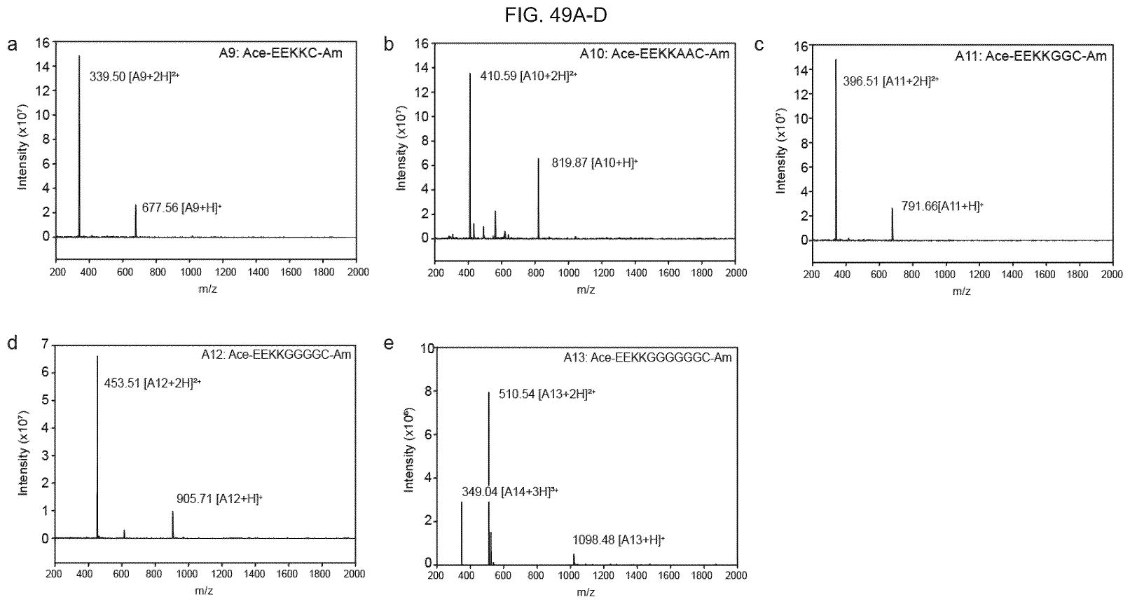

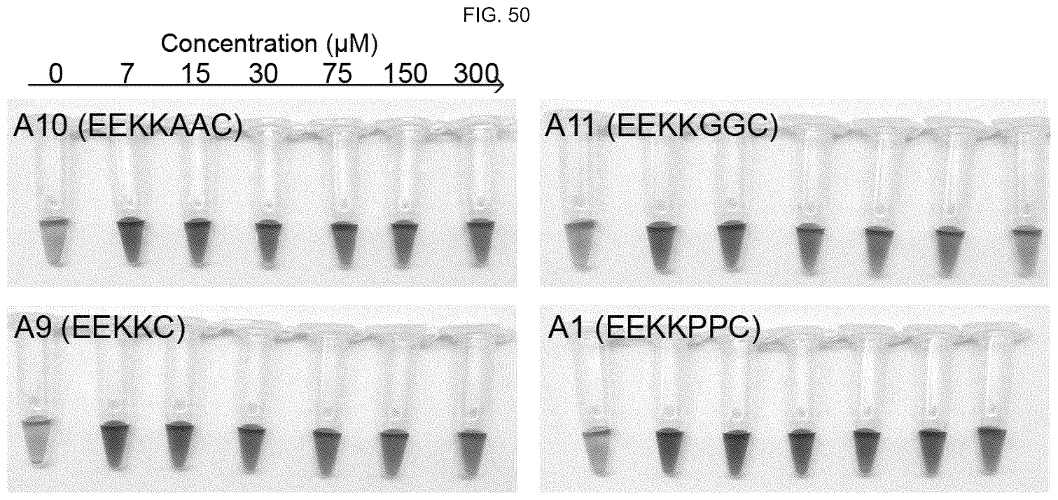

Pro-, Ala-, and Gly-based spacers have different hydrophobic and hydrophilic natures, rigidity, and flexibility.^{22,29} For example, Pro residue is more hydrophobic and rigid (i.e, low mobility) while Gly residue is more hydrophilic and flexible. To investigate the impact of the rigidity and hydrophilicity of the peptides on the particle dissociation, we synthesized A1, A10, and A11 peptides that have different spacers: PP, AA, GG (FIG. 38a-b and FIG. 49). We also synthesized the peptide without a spacer (i.e., A9) to confirm the impact of the spacer on particle dissociation. The results showed that the A1, A9, A10, and A11 peptides dissociated AuNP aggregates, changing color from blue to red within 10 min, respectively (FIG. 38c-d and FIG. 50). The A11 peptide showed the highest dissociation capacity compared to other spacers (FIG. 38e). For example, the CDC of A11, A1, and A10 peptides were 16 μM, 40 μM, and 150 μM, respectively, indicating that higher hydrophilic and flexible spacer could enhance dissociation process. We further compared colloidal stability of the dissociated AuNPs by A1, A9, A10, and A11 peptides. The dissociated AuNPs were incubated in NaCl (from 0.5 to 2 μM) for 1 h and measured variations of absorbance between 520 nm and 600 nm. The A9-capped AuNPs (i.e., without spacer) showed relatively low colloidal stability compared to Pro-, Ala-, and Gly-capped AuNPs (FIG. 38f). Pro-capped AuNPs showed a two-fold lower aggregation parameter than Gly- or Ala-capped AuNPs possibly due to their rigid structure and SAM.²² Lastly, Fourier-transform infrared spectroscopy (FTIR) data confirmed peaks at 1600 cm⁻¹ and 1400 cm⁻¹ attributed to carboxyl groups in Glu amino acid (FIG. 38g).

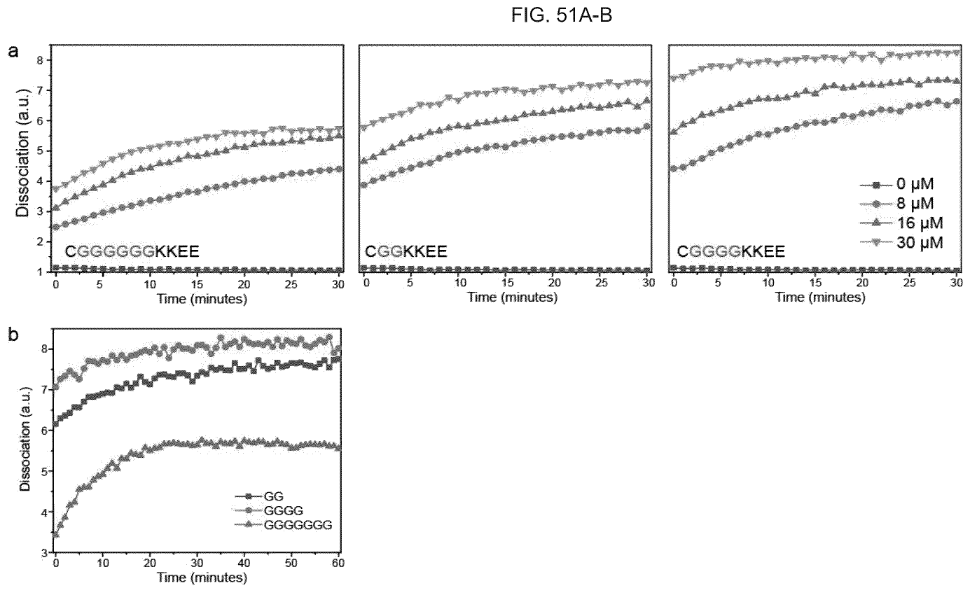

Next, we examined the impact of the peptide length on the particle dissociation. Gly spacer was selected because it showed the highest dissociation capacity compared to the Pro- and Ala- spacers. The A11, A12, and A13 peptides which contain two, four, and six repeated Gly amino acid were synthesized for the test (FIG. 38b). The average peptide length of four repeated Gly (GGGG) (SEQ ID NO:39) is known around 18-20 Å.²² The particle dissociation was improved as a function of the increased number of Gly spacer (from two to four) (FIG. 38h and FIG. 51A-B). The A11 and A12 peptides dissociate AuNP aggregates with the concentration of 16 μM, respectively. However, the A13 peptide showed 25% lower dissociation capacity than the A12 peptides at the same peptide concentration (30 μM). These results revealed that the particle dissociation relies on the peptide length: the spacer length until 20 Å improved particle dissociation while over 20 Å could inversely impact the dissociation capacity likely due to steric hinderance caused by the large size of the spacer.¹⁴

FIG. 38: Impact of hydrophilicity and steric bulk on particle dissociation. a, Different Pro-, Ala-, Gly-spacers have different nature of rigidity and hydrophilicity which can impact on the dissociation capacity. Table 2 in (b) describes peptide sequences that are designed to investigate the impact of spacers. c, Photographs of the dissociated AuNPs by the PP, AA, GG spacers and without spacer (−) as a negative control. d, Time-dependent particle dissociations driven by the A1, A9, A10, and A11 peptides, respectively. e, Gly spacer showed the higher dissociation capacity than the Pro- and Ala- spacers. f, Aggregation parameter of the dissociated AuNPs driven by the A1, A9, A10, and A11 peptides. The results showed that the peptide with spacer can provide higher colloidal stability for AuNPs than the peptide without spacer. g, FTIR data of the dissociated AuNPs by the A1, A9, A10, and A11 peptides. The peaks at 1400 cm⁻¹ and 1600 cm⁻¹ were attributed to carboxyl group in Glu amino acid. h, Impact of the spacer length on the particle dissociation. Increasing the length of the spacer (from two to four) improved dissociation capacity while the spacer with six Glu (i.e., A13) showed lower dissociation capacity than A12 peptide. The panel e, f, and h repeated three independent times and showed similar results.

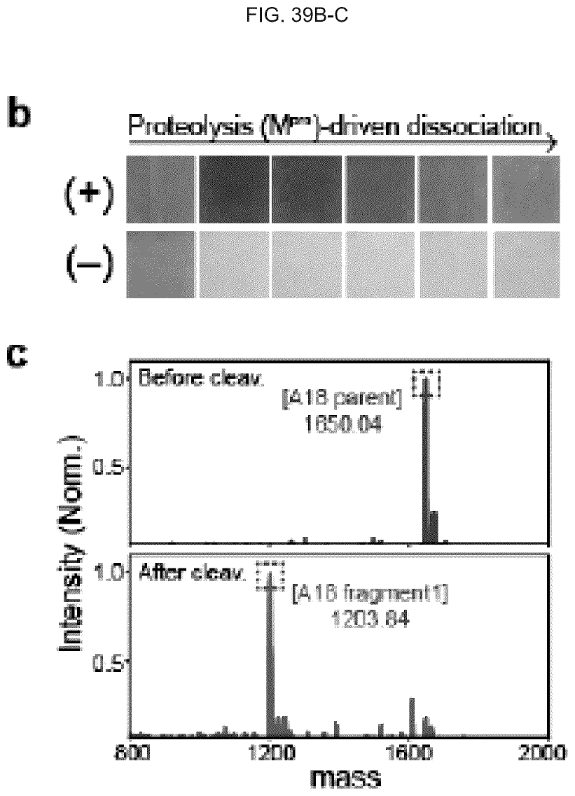

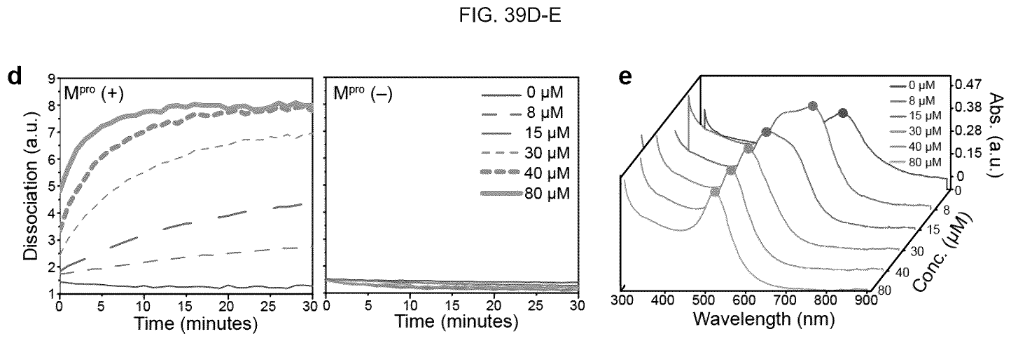

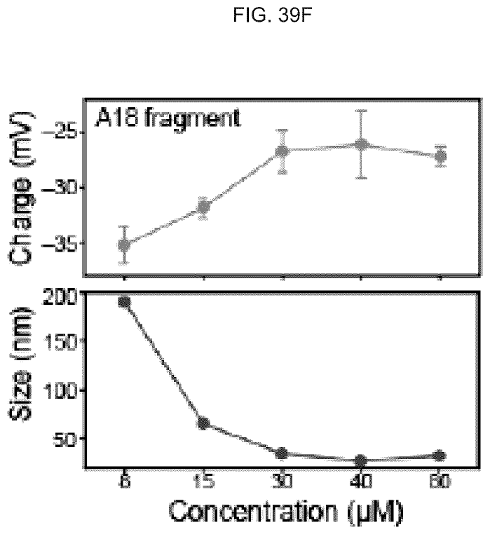

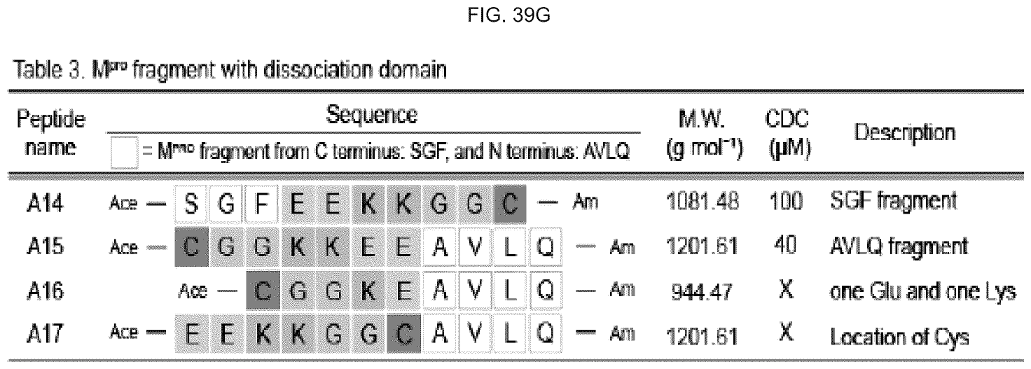

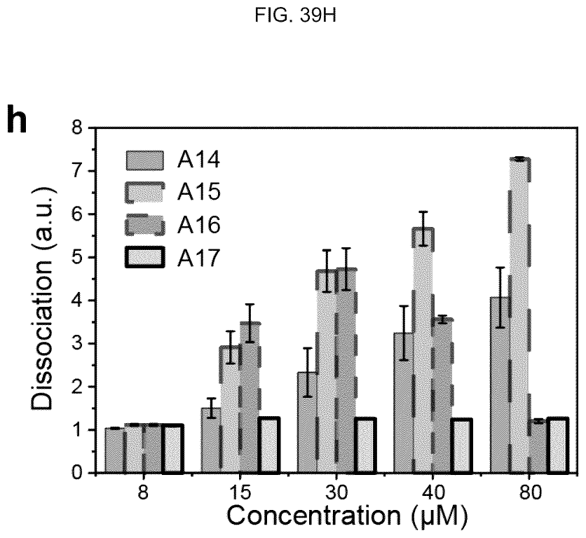

Protease detection with dissociation peptide. We then applied this exemplary dissociation strategy for M^{pro} detection.^{17,18} We used the A18 peptide which contains three major structural components: dissociation domain (CGGKKEE (SEQ ID NO:26) at the N terminus), cleavage site (AVLQJSGF), and one Arg at the C terminus for dissociation shielding according to the A4 peptides (FIG. 39a). The A18 fragments released by M^{pro} in PB buffer dissociated AuNP aggregates, changing color from blue to red while the A18 peptides without M^{pro} showed no color changes and became transparent due to the colloidal settlement (FIG. 39b). Matrix assisted laser absorption ionization time of flight mass spectrometry (MALDI-TOF MS) data confirmed the mass peaks of the A18 fragments (CGGKKEEAVLQ (SEQ ID NO:27): 1203.84) which is a result of the M^{pro} proteolysis (FIG. 39c). After M^{pro} incubation (200 nM) for 1 h, the A18 fragments with different concentrations from 8 to 80 μM were used for the dissociation. The result showed that at least 40 μM of the A18 peptide (CDC) was required to dissociate AuNP aggregates, and the dissociation quickly occurred within 10 min (FIG. 39d). The plasmonic resonance peak of AuNP aggregates blue shifted to 520 nm which was an absorption peak of pristine AuNPs. (FIG. 39e). The size of AuNP aggregates was reduced to 26±0.30 nm, and the surface charge was −27±0.95 mV as a function of the released A18 fragments by M^{pro} cleavage (FIG. 39f).

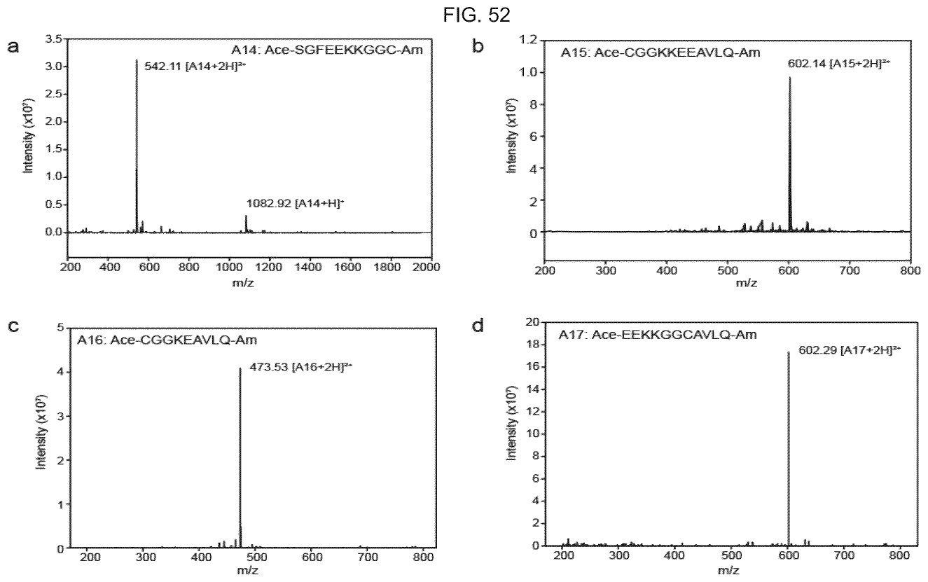

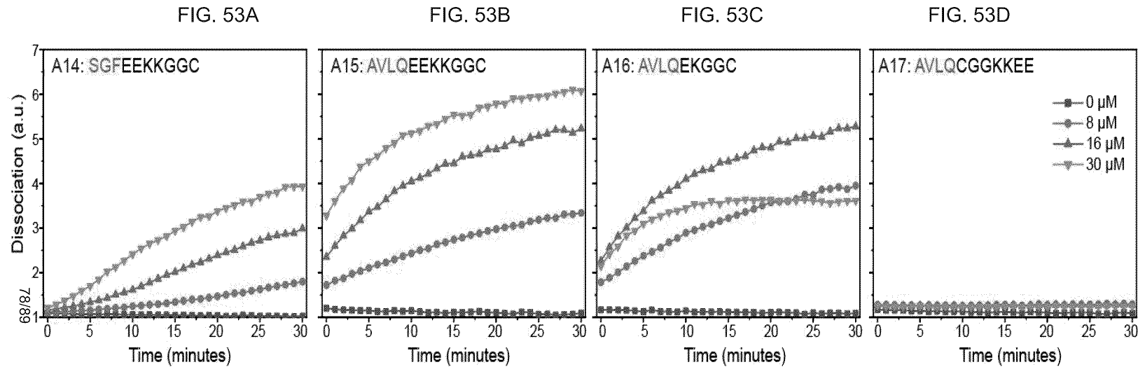

Next, we synthesized four different peptide sequences to study the impact of the fragments (for example, SGF or AVLQ (SEQ ID NO:28)), charge density, and the location of Cys residue on the particle dissociation (FIG. 39g and FIG. 52A-D). M^{pro} cleavage requires an AVLQJSGF site (M^{pro} cleaves after Arg), and the two different fragments (for example, AVLQ or SGF) could impact on the dissociation capacity of the peptide. To decide the location of the dissociation domain, we synthesized a dissociation domain with AVLQ (SEQ ID NO:28) (i.e., A15) and SGF (i.e., A14), respectively. The A15 peptide showed higher dissociation capacity than the A14 peptide, indicating that adding the dissociation domain at the N-terminus is a better approach than adding at the C-terminus (FIG. 39h and FIG. 53).

For FIG. 53: Time-dependent particle dissociations using the A14, A15, A16, and A17 peptides, respectively. Mpro fragments (which are SGF and AVLQ) could attenuate the dissociation efficiency. The SGF fragment more reduced the dissociation capacity than AVLQ fragment likely due to strong hydrophobicity of Phe (F) amino acid. It is notable that the location of Cys is also important to dissociate AuNP aggregates. For example, the Cys at the N-terminus (i.e., A15) dissociated AuNP aggregates while the Cys in the middle (i.e., A17) failed to dissociate AuNP aggregates. This is likely because ligand architecture could impact on grafting density of peptide on AuNPs. The Cys in the middle could have less passivation layer which prevents particle dissociation.⁸

In addition, the A17 peptide which has lower charge components decreased the dissociation capacity, and the location of the Cys amino acid can impact particle dissociation. For example, the Cys at the N terminus showed 5-fold higher dissociation capacity than the peptide with Cys in the middle as Cys in the middle gives less passivation of AuNPs, thus preventing particle dissociation.²² In conclusion, we placed this exemplary dissociation domain (i.e., CGGKKEE) (SEQ ID NO:26) at the N terminus to efficiently activate particle dissociation by M^{pro} proteolysis.

FIG. 39: M^{pro} detection using dissociation strategy. a, Schematic illustrates that M^{pro} cleavage releases dissociation domains, changing the color from blue to red. This exemplary dissociation peptide (i.e., A18) comprises three parts: dissociation domain (CGGKKEE) (SEQ ID NO:26), cleavage site (AVLQJSGF), and dissociation shielding site (R). The inset images are before and after particle dissociation obtained by darkfield microscopy. Blue dots indicate actual AuNP aggregates (left) and the dissociated AuNPs (right). b, Color changes with (+) and without (−) M^{pro} in PB buffer. The released A18 fragment (CGGKKEEAVLQ) (SEQ ID NO:27) dissociated AuNP aggregates, changing the color from blue to red. c, MALDI-TOF MS data before and after M^{pro} cleavage, confirming the mass peaks of the A18 parent and its fragment. d, Time-dependent particle dissociation by the A18 fragments. The results showed that at least 40 μM of the A18 fragment was required for particle dissociations. e, UV-vis spectrum before and after particle dissociation by the A18 fragments with different concentrations (8-80 μM). f, Changes in the size and surface charge after the dissociation induced by M^{pro} cleavage. Table 3 in (g) describes peptide sequences that are designed to confirm the best location and order of the dissociation domain for M^{pro} detection. h, Particle dissociations driven by the A14, A15, A16, and A17 peptides. The results show that the dissociation domain located at C-terminus showed the highest dissociation capacity. In addition, the thiol group at the tail showed higher dissociation affinity than thiol group in the middle. The panel f, and h repeated three independent times and showed similar results.

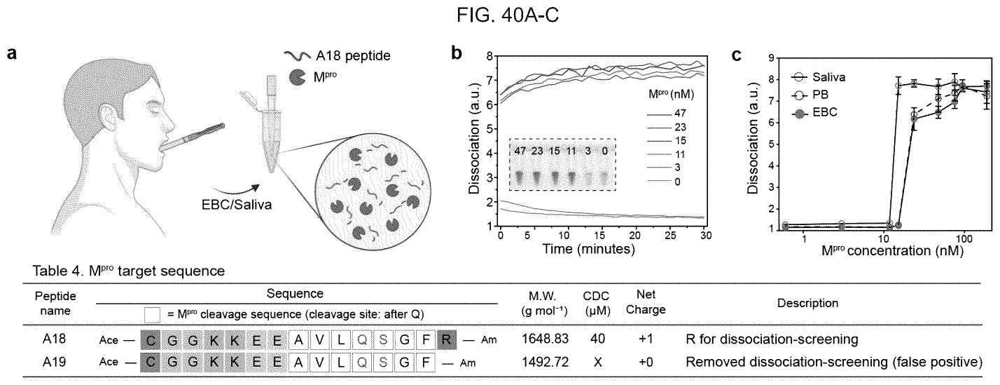

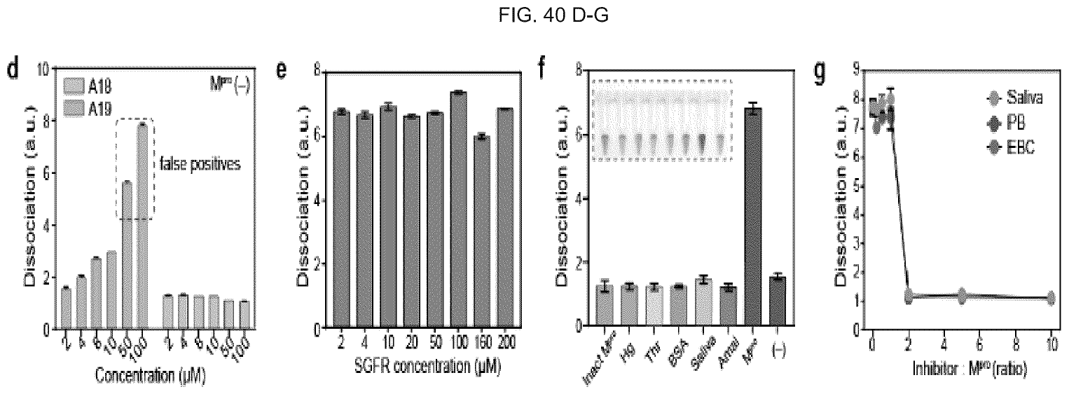

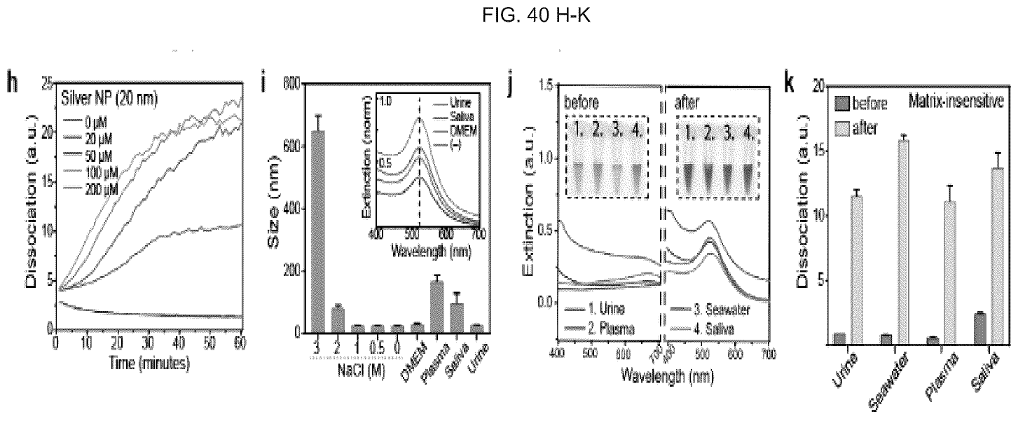

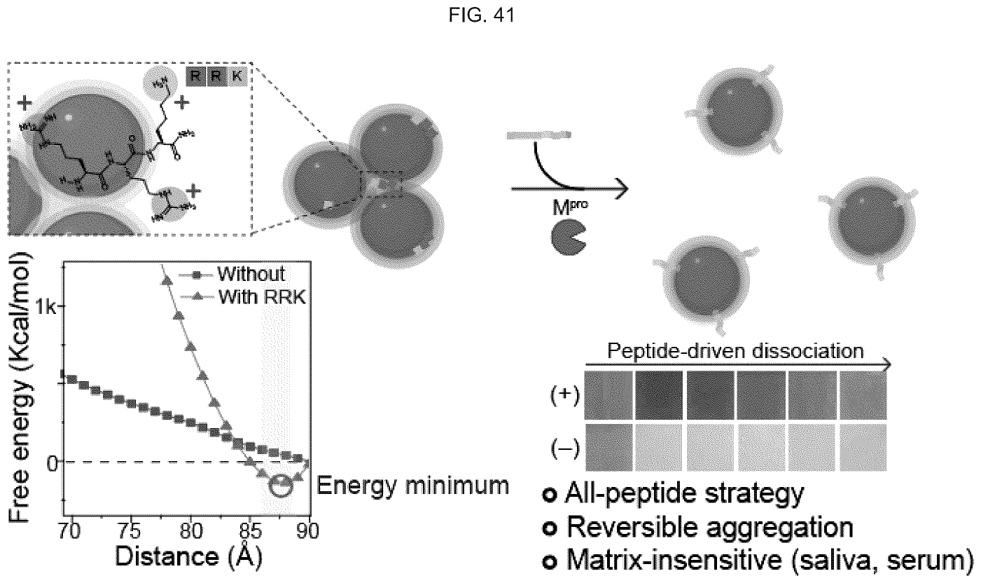

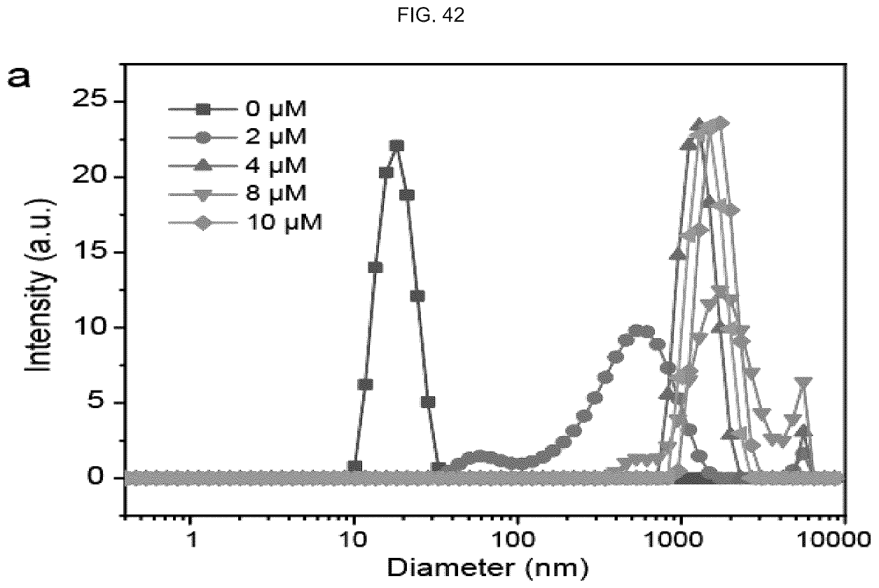

Matrix-insensitive M^{pro} detection. This exemplary dissociation strategy offers a matrix-insensitive target detection because the dissociation mechanism is less interrupted by operating mediums (for example, proteins, ions) compared to aggregation-based biosensors.¹⁴ Since SARS-CoV-2-infected patients might release viral proteases to respiratory fluids,³⁰ the exemplary A18 peptides were examined to dissociate AuNP aggregates in saliva or EBC (FIG. 40a). We performed a stepwise assay by first incubating the A18 peptide with different M^{pro} concentrations (0.3 to 47 nM) for 30 min at 37 C°. Then, AuNP aggregates were added as a readout for 1 h. FIG. 40b showed that the A18 fragments cleaved by M^{pro} in saliva or EBC dissociated AuNP aggregates within 10 min. FIG. 40c plots the ratiometric signal (λ₅₂₀/λ₇₀₀) against different M^{pro} concentrations (from 0.6 to 150 nM), showing that higher M^{pro} concentrations quickly activated particle dissociation than at lower concentrations. The LoD for M^{pro} was determined to be 12.3 nM in saliva, 16.7 nM in the EBC, and 16.4 nM in PB buffer, respectively. The CDC of the A18 fragment was 40 μM. When the concentration of the A18 fragment was above CDC, the color turned to red. Otherwise, the color became transparent due to colloidal settlement in saliva or EBC (FIG. 20).

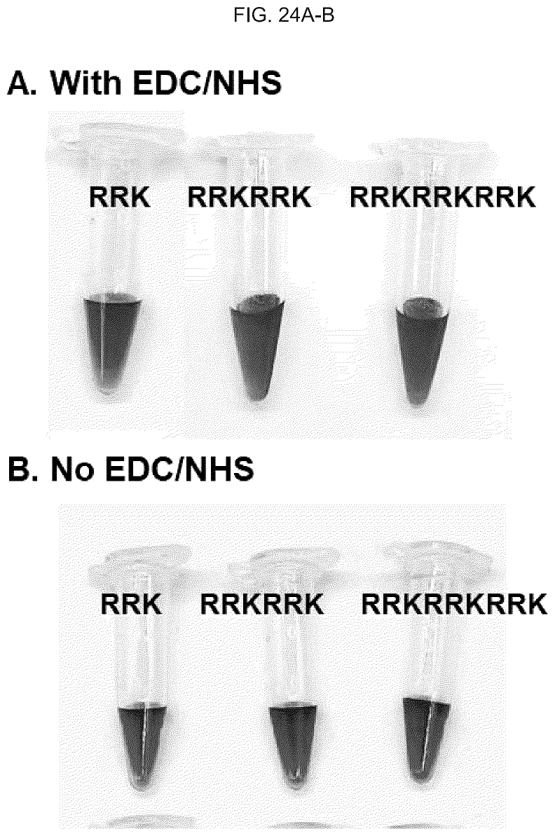

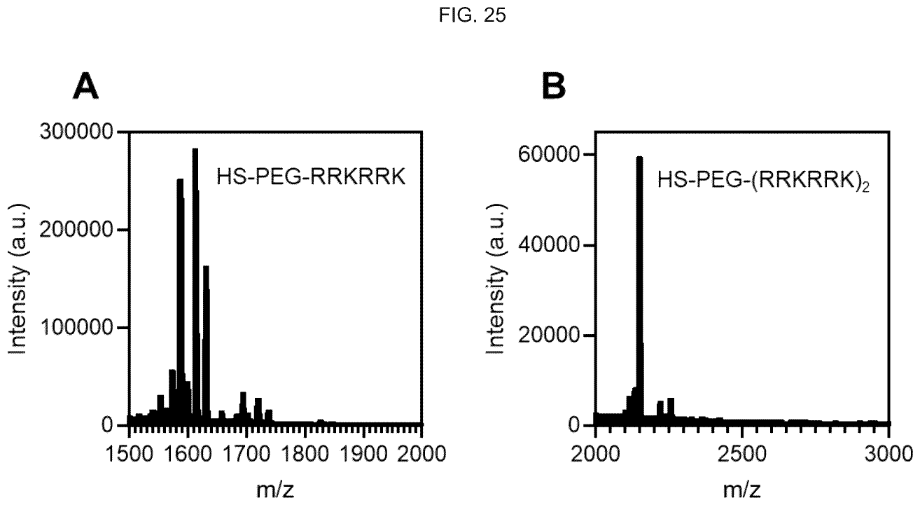

To prevent non-desired particle dissociation, a positively charged domain was placed at the C terminus for dissociation-screening: one R in A18 and no R in A19 as a negative control (Table 4 in FIG. 40). In the absence of M^{pro}, A19 peptide over 50 μM can cause non-desired particle dissociation while A18 peptide showed no false positives (FIG. 40d). We further confirmed that the release of SGFR (SEQ ID NO:31) fragments during M^{pro} proteolysis had negligible impact on the dissociation process (FIG. 40e). We conducted a specificity test for M^{pro} using several related proteins such as hemoglobin (hg), inactivated M^{pro} (incubated at 60 C° for 3 h), thrombin (thr), bovine serum albumin (BSA), human saliva, and α-amylase (amal). FIG. 40f shows that only the positive control (i.e., 200 nM M^{pro}) produced a prominent optical signal due to the release of the dissociation peptide by proteolytic cleavage. To define the enzymatic role in the protease in the colorimetric assays, a competitive inhibitor (GC376) for M^{pro} was used for the test. M^{pro} (200 nM) was incubated with increasing molarity of GC376 (i.e., 0-2 μM) in different operating mediums such as saliva, EBC, and PB buffer for 10 min prior to adding the A18 substrates. FIG. 40g indicates that particle dissociation was prevented by the addition of inhibitors (from 400 nM to 2 μM) due to the formation of M^{pro}-GC376 complexes (FIG. 24 and FIG. 25).

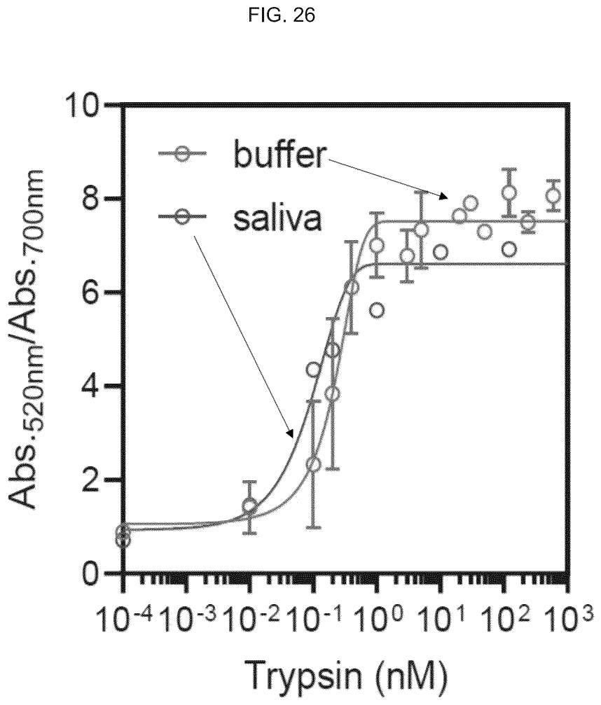

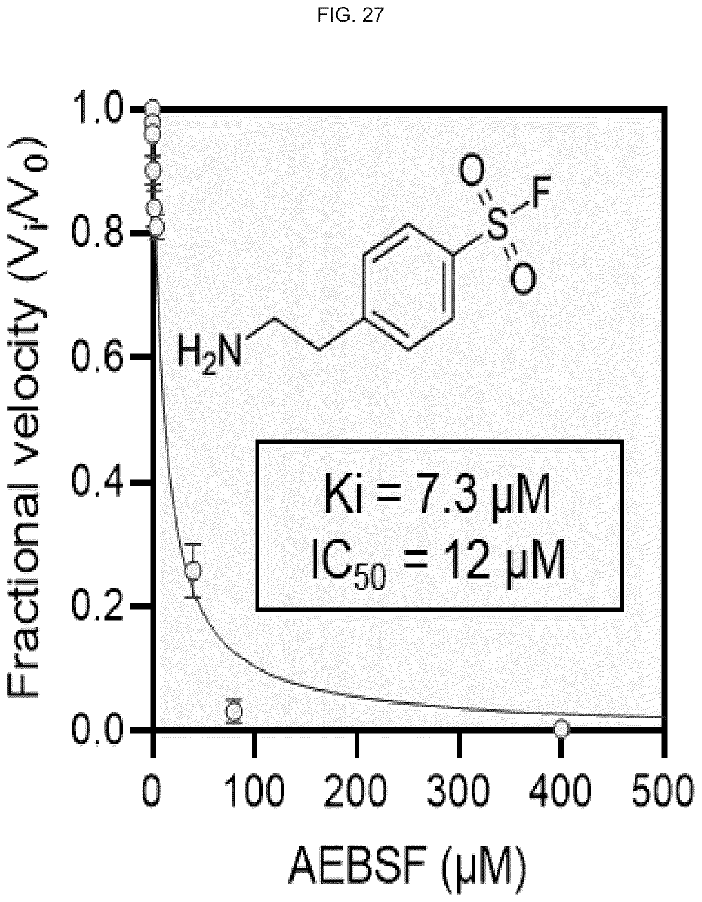

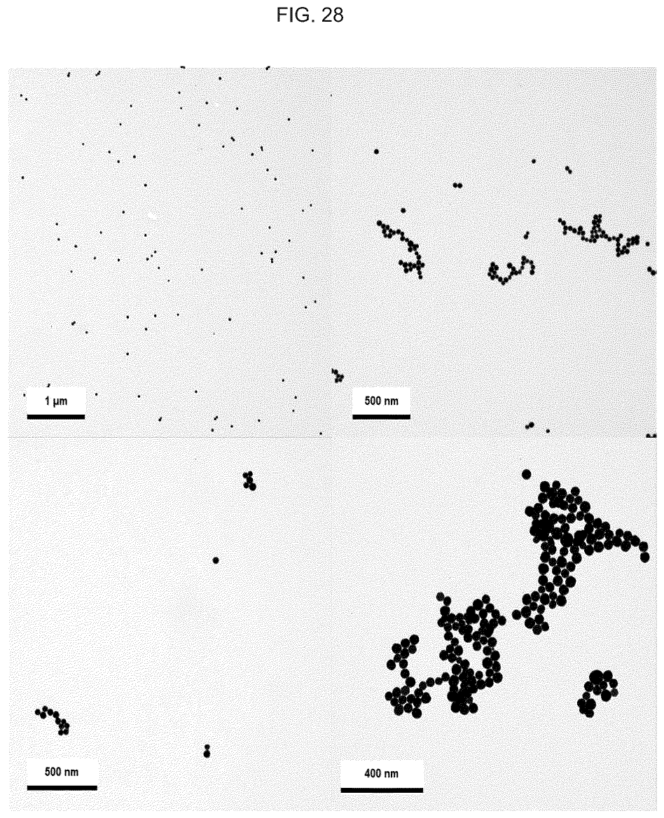

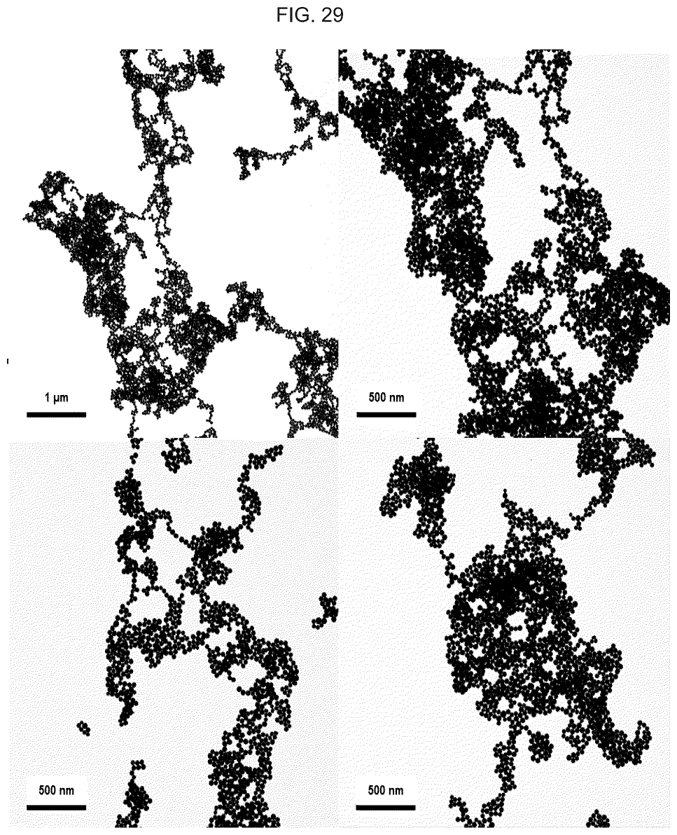

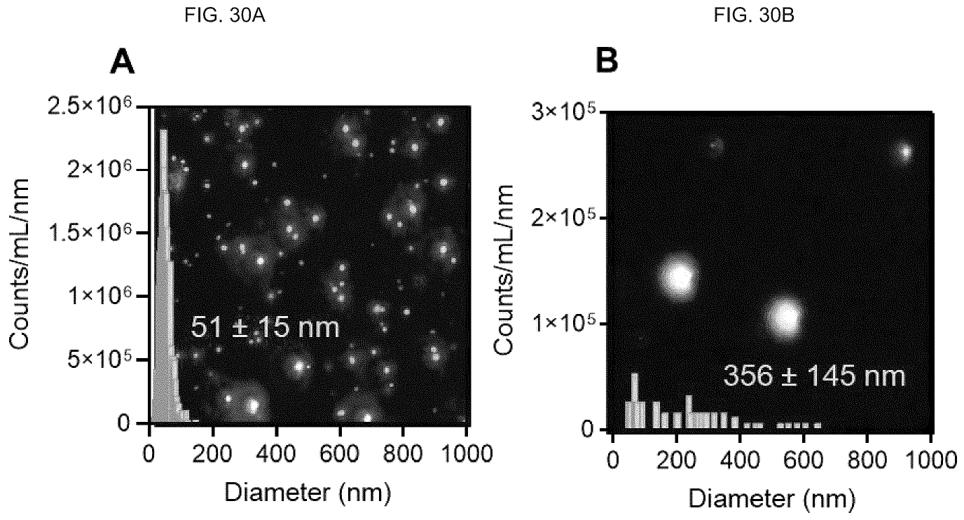

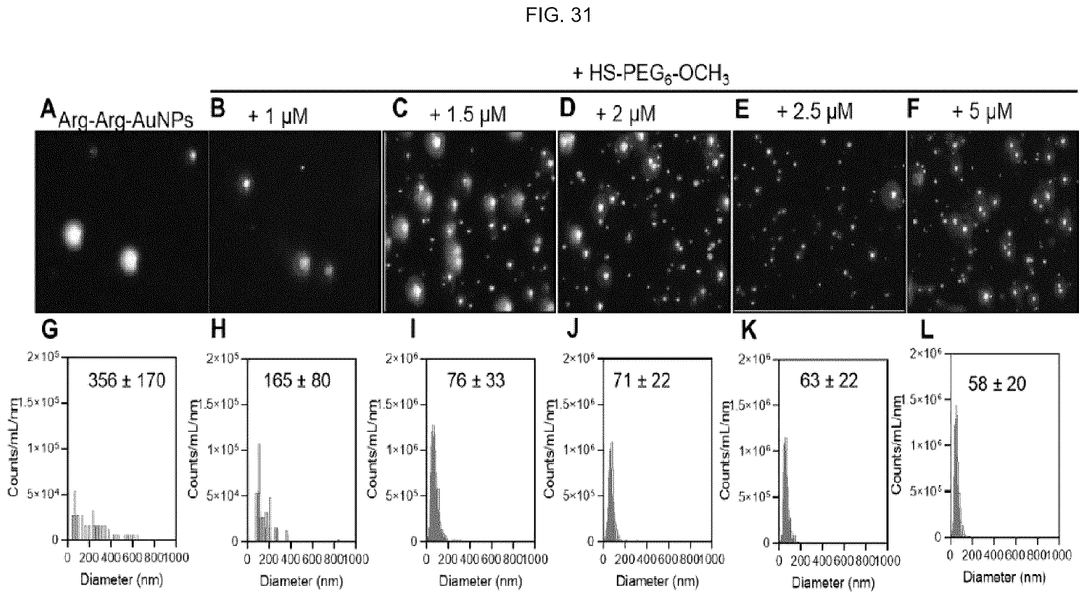

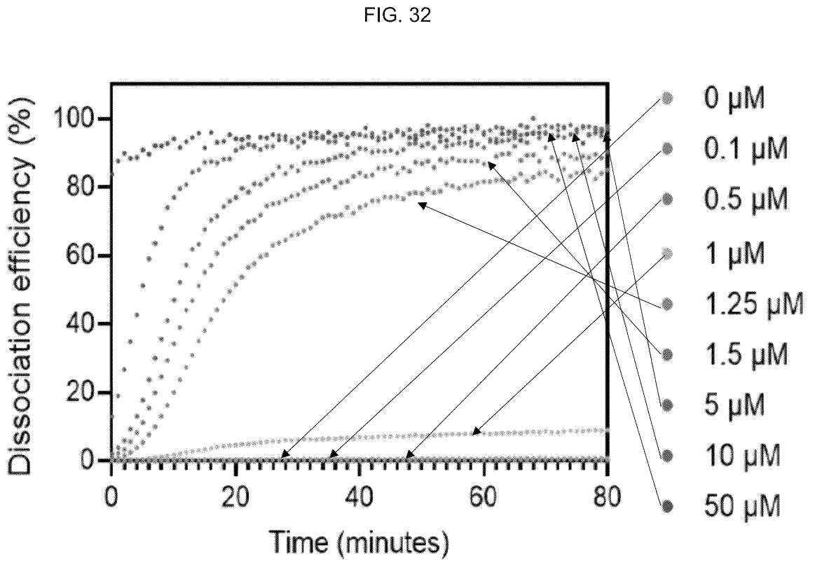

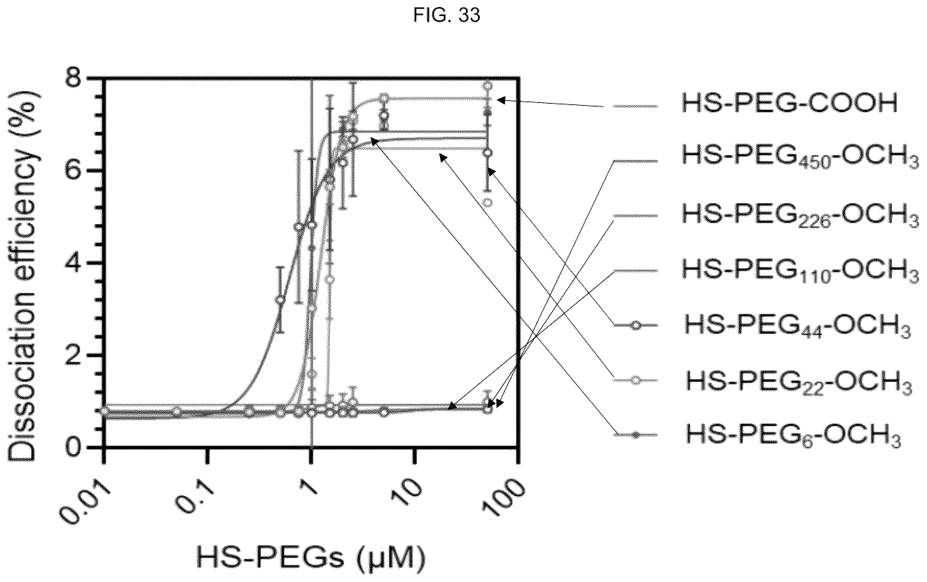

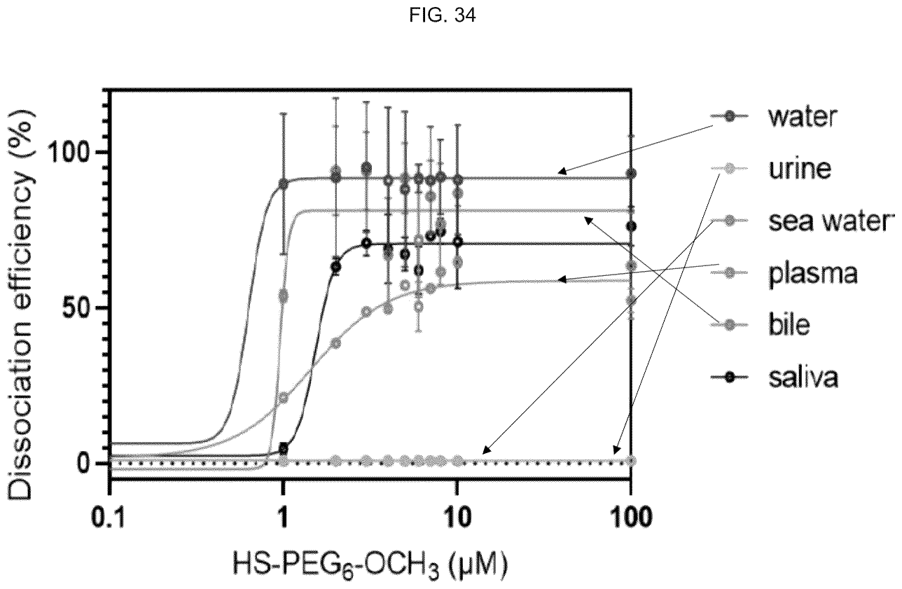

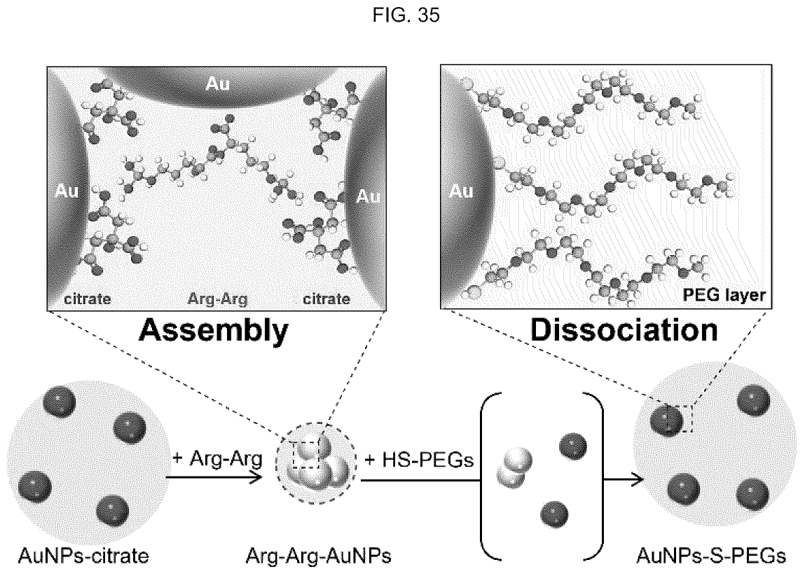

Next, we applied this exemplary dissociation strategy on silver nanoparticles (AgNPs, 20 nm in size) because AgNPs offer a higher order of extinction coefficient compared to AuNPs.³¹ As expected, the release of the A18 fragments by M^{pro} cleavage could dissociate AgNP aggregates, changing color from blue to yellow (FIG. 40h and FIG. 26). This exemplary dissociation strategy can also improve colloidal stability of plasmonic nanoparticles. After the particle dissociation, the A18-capped AuNPs maintained high colloidal stability in extreme conditions such as Dulbecco's modified eagle medium (DMEM), human plasma, saliva, human urine, and NaCl (from 0.5 to 2M) (FIG. 40i). Lastly, this exemplary dissociation strategy is less affected by matrix interference. We replaced the sample matrix with 100% human plasma, urine, saliva, and seawater after RRK-based AuNPs aggregation. After adding dissociation peptides, reversible aggregation still occurred, leading to a blue-shift of the plasmonic resonance peak; this in turn changes color from blue to red in 100% human plasma, urine, saliva, and seawater (FIG. 40j-k and FIG. 27). These results emphasize that this exemplary peptide-based dissociation strategy provides a simple and versatile approach for reversible aggregation of plasmonic assemblies, offering a new mechanism for designing a matrix-insensitive plasmonic biosensor.

FIG. 40: Matrix-insensitive M^{pro} detection: a, Schematic illustration of a matrix insensitive M^{pro} detection. The released A18 fragment by M^{pro} cleavage was used for colorimetric biosensing in saliva or EBC. b, Time-dependent M^{pro} detection from 0 to 47 nM in saliva. The inset photograph shows that this exemplary dissociation strategy can provide a clear readout of positive M^{pro} sample above 11 nM in saliva. c, Detection limits of M^{pro} in saliva, EBC, and PB buffer, respectively. Table 4 describes peptide sequences that are designed to verify the role of dissociation screening domain. d, One Arg at the C-terminus can prevent false positives. False positives occurred in A19 when the peptide concentration was over 50 μM while A18 showed no false positive in the absence of M^{pro}. e, A18 fragment from C terminus (i.e., SGFR (SEQ ID NO:31)) had negligible impact on the dissociation process. f, Specificity test using multiple different biological essays (for example, inactivated M^{pro} (inact M^{pro}), hemoglobin (Hg), Thrombin (Thr), BSA, Saliva, Amalyase (Amal)). g, GC376 inhibitor assay test in saliva, EBC, and PB buffer, respectively. h, The released A18 fragments by M^{pro} cleavage can dissociate other types of plasmonic assemblies such as AgNP aggregates. i, After particle dissociation, the A18-capping AuNPs maintained high colloidal stability in different biological (for example, urine, saliva, plasma, DMEM) and extreme condition (for example, 2M NaCl). j, The plasmonic resonance peaks of the AuNP aggregates blue-shifted after particle dissociation in 100% of 1. human urine, 2. plasma, 3. seawater, and 4. saliva. The inset photographs show before (left) and after (right) adding dissociation peptides. k, Ratiometric signal (λ₅₂/λ₇₀₀) of the dissociated AuNPs in diverse matrixes, indicating that this exemplary dissociation strategy is less affected by sample matrix. The panel c, d, e, f, g, i, j, and k repeated three independent times and showed similar results.

### Discussion

In summary, we developed peptide-driven dissociation of plasmonic assemblies as a response to M^{pro} detection of SARS-CoV-2. This strategy eliminated the need for surface modifications of AuNPs and complex couplings (for example, PEG-peptide) for protease sensing. Both computational and experimental methods were used to understand reversible aggregation by a short cationic RRK peptide. Using 19 different peptide sequences, we verified that the dissociation capacity relies on hydrophilicity, charge density, ligand architecture, and steric distance. After incorporating the dissociation domain with an M^{pro} cleavage site, a colorimetric assay using UV-vis spectroscopy was tested to confirm the reproducibility and applicability of platform for M^{pro} detection.

With optimized peptide sequence, our dissociation strategy successfully produced a distinct optical signal as a function of the released peptides by M^{pro} cleavage with a detection limit of 12.3 nM in saliva. The dissociation-screening site at C terminus enhances proteolytic cleavage (around 3-fold)³² and prevents false positives. Inhibitor assay and specificity test further confirmed the critical role of M^{pro} in dissociation process, showing no non-specific activation. The dissociated AuNPs maintained high colloidal stability in extreme conditions, and this exemplary dissociation strategy can be applied to other types of plasmonic materials such as silver. We further demonstrated that this exemplary dissociation strategy can be less interrupted by matrixes such as human plasma, urine, and seawater. This peptide-driven dissociation strategy holds significant promise in various fields such as colloidal science, biochemistry, and plasmonic biosensors with diverse applications ranging from disease diagnosis and drug detection to environmental monitoring.

### Methods

### Experimental Details

### 1.1 Preparation of AuNPs and AuNP Aggregates

Citrate-stabilized AuNPs with the size of 13 nm was synthesized using the Turkevich method.³³ Briefly, 45 mg of HAuCl₄·3H₂O was dissolved in the 300 mL of MQ water under the generous stirring (600 rpm) and boiling condition at 120° C. Then, 150 mg sodium citrated (dissolved in 5 mL of MQ water) was rapidly injected, and the reaction was left under boiling conditions for 20 min. The color of solution changed from purple to gray and dark reddish. The resulting product was cooled down and was stored at room temperature for the future use. The optical density of the final product was 1.45 (concentration approximately 3.6 nM, ε₅₂₀=4.0×10⁸ M⁻¹cm⁻¹). Notably, 500 mL of round flask was cleaned with acua regia and distilled water (three times) before the synthesis.

Briefly, 10 μM of RRK peptide was used to aggregate 100 μL of AuNPs (conc approximately 3.6 nM). The particle aggregation rapidly occurred (less than 10 s), changing color from red to blue. Then the desired dissociation peptides were used to dissociate the AuNP aggregates. It is notable that these exemplary AuNPs are stabilized by citrate. Different surface ligands (for example, BSPP) and different sizes (40 or 60 nm) require different amounts of RRK peptide for particle aggregation. Surface ligands with large molecular weight (for example, PEG_{1 k}, or PVP_{55 k}) are difficult to aggregate, indicating that PEG or PVP-stabilized AuNPs are not suitable for the dissociation strategy.

### 1.2 Dissociation of AuNP Aggregates Using Proteolysis of Peptides

Briefly, a dried peptide powder was dissolved in phosphate buffer (20 mM, pH 8.0) and incubated with the M^{pro} at a molar ratio of 3000:1 (substrate: enzyme ratio) for 0.5 h at 37° C. To confirm the M^{pro} cleavage site, the sample was purified using a C18 column (5 μm, 9.4×250 mm) and eluted with a flow rate of 3 mL/min over 30 min with a linear gradient from 10% to 95% to ACN in H₂O. After the purification, the molecular weight of a fragment peptide was confirmed by using ESI-MS (positive or negative mode) or/and matrix (for example, HCCA) assisted laser desorption ionization time of flight mass spectrometry (MALDI-TOF MS, Bruker Autoflex Max) in the Molecular Mass Spectrometry Facility at UC San Diego.

To test particle dissociation in saliva and EBC condition, the desired amounts (conc, 30 μM) of the dissociation peptides (A18, Ace-CGGKKEEAVLQSGFR-Am (SEQ ID NO:30)) were incubated with M^{pro} in the 100% of saliva or EBC for 0.5 h at 37° C. Then, the 40 μL of A18 fragment peptides in saliva or EBC were mixed with 100 μL of AuNP aggregates for colorimetric sensing. The mass peak of the A18 peptide and its fragment was confirmed by MALDI-TOF MS examination, respectively.

### 1.3 Dissociation Strategy in Diverse Matrixes

Briefly, RRK peptides (8-10 μM) was first used to trigger AuNP aggregation in distilled water. The sample was centrifuged at 1 g for 5 min to remove the supernatant. Then, the pellet was re-dispersed in 100% human saliva, plasma, urine, and seawater. After re-dispersion, the desired amounts of dissociation peptide were used to dissociate AuNPs aggregates in different matrixes. Both A11 and A12 peptides successfully dissociated the aggregated AuNPs in diverse matrixes. The experiment was performed with three replicates, and the microplate reader was used to measure spectral scanning from 300 to 900 nm before and after dissociation. The ratiometric signal (λ₅₂₀/λ₇₀₀) was referred as dissociation. The data was blanked to remove background signal.

### Computational Details

### 2.1. Investigation of RRK Interaction on a Citrate-Coated AuNP Using MD Simulations

The forcefields applied in MD simulations were based on AMBER³⁴ forcefield (RRK, citrate, Na, Cl), TIP3P³⁵ forcefield (water), and EAM/Fs potential (Au) from Ackland et. al.³⁶ The pair interactions were determined by general mixing rule except the RRK|Au and citrate|Au interactions, which were constructed based on the parameterization of QM interaction energies. In MD simulations, a long-range particle-particle particle-mesh solver, Van der Waals cutoff 10 Å, timesteps 1.0 fs, and SHAKE algorithm³⁷ for water molecules and Hydrogen atoms were adopted.

To investigate the binding phenomenon for RRK molecules toward a citrate-coated AuNP system, we performed MD simulations using LAMMPS³⁸ engine. The initial structure contained a 5 nm-diameter Au nanoparticle, 80 citrate molecules, 240 Na ions, and 4670 water molecules. A MD simulation was initiated with 500 conjugated gradient steps, followed by the canonical ensemble (NVT) to heat up a system into 298K. Afterwards, the isobaric/isothermal ensemble (NPT) was proceeded to optimize systemic density at 298K/1 atm and NVT ensemble was further applied to equilibrate a system. Based on the equilibrated citrate-coated Au nanoparticle structure, furthermore, we constructed a citrate|RRK Au nanoparticle system by randomly placing 80 RRK molecules and 240 Cl ions around the citrate-coated Au nanoparticle and embedding it into water solvents, thereby a structure with AuNP|80 citrate∥240 Na|80 RRK|240 Cl|33622 water was constructed. With the same procedure as mentioned in this section, an equilibrated AuNP180 citrate|240 Na|80 RRK|240 Cl|33622 water system was obtained, demonstrating the RRK binding phenomenon.

### 2.2. Determining Free Energies as a Function of Nanoparticle Distance Using Steered Molecular Dynamics (SMD) Simulations

SMD simulations were performed to investigate free energy values as two Au nanoparticles approached each other. In this study, we adopted the same forcefield parameters, long-range solver, cutoff point, and SHAKE algorithm as mentioned in MD simulation section. There were two systems performed: a system without RRK molecules and a system with RRK molecules, where the first system contained two 5 nm-diameter Au nanoparticles, 870 citrate, 2160 Na, and 29238 water, and the second system contained two 5 nm-diameter Au nanoparticles, 870 citrate, 2160 Na, 95RRK, 285Cl, and 45542 water. Each model was initially equilibrated using the same procedure as MD simulation and further performed a 3.7 ns SMD simulation to investigate free energies as a function of Au nanoparticle distance. In a SMD simulation, we adopted a harmonic restraint with a force constant 100 kcal/mol·Å,² where the equilibrium value of the harmonic restraint was gradually changed from 91 Å into 55 Å, and saved free energy values as two Au nanoparticles approached each other, thereby the free energy values at different Au nanoparticle distance were determined.

### 2.3. Free Energy Investigation Using Metadynamics Approach

To explore the molecular behavior when a molecule approaches to an Au(111) surface, we constructed systems with periodic boundaries in x, y coordinates and a finite boundary in z coordinate, and explored free energy values using Metadynamics (MTD) approach.⁶ Two systems were constructed: (1) a single citrate molecule on an Au(111) slab, representing as the procedure to form a citrate-coated Au surface, and (2) a single RRK molecule on a citrate-coated Au surface, representing as the procedure for adding RRK molecules into a citrate-coated Au system. The corresponding number of Na⁺(Cl⁻) ions were added to form a charge neutral system and the system cell size was (57.48778 Å, 49.78588 Å, 150 Å) in (x, y, z). Au(111) slab position was fixed at 19 Å/31 Å (bottom/top) and the slab-slab interactions were turned off via inserting empty volume in z with a factor 2.0.

Each system was initialized using 500 steps CG minimization and further heated up into 298K using Nose-Hoover thermostat (NVT ensemble). Afterwards, ins NVT ensemble was adopted to equilibrate a system. In Metadynamics section, 50 ns trajectory was proceeded, and the z coordinate was measured as the center of mass of the citrate molecule in (1) system and the center of mass of the RRK molecule in (2) system, where Gaussian functions with a weight of 1.0 kcal/mol and a width 1.25 Å were deposited every 0.2 ps into each system. As the results (FIG. S3), a weaker peak with a relatively far distance from an Au (111) surface was found in (2) system, implying a RRK molecule interacted with citrates and bound on a citrate-coated Au surface, which reduced the charge effect on Au surfaces and further resulted in the NP aggregation phenomenon.

### REFERENCES FOR EXAMPLE 1

(1) Liu, X.; et al. Extinction Coefficient of Gold Nanoparticles with Different Sizes and Different Capping Ligands. Colloids SurfB Biointerfaces 2007, 58 (1), 3-7. https://doi.org/10.1016/j.colsurfb.2006.08.005.
(2) Pramanik, A.; et al. The Rapid Diagnosis and Effective Inhibition of Coronavirus Using Spike Antibody Attached Gold Nanoparticles. Nanoscale Advances 2021, 3 (6), 1588-1596. https://doi.org/10.1039/d0na01007c.
(3) Saha, K.; et al. Gold Nanoparticles in Chemical and Biological Sensing. Chem. Rev. 2012, 112 (5), 2739-2779. https://doi.org/10.1021/cr2001178.
(4) Gosselin, B.; et al. Ultrastable Silver Nanoparticles for Rapid Serology Detection Of. Analytical Chemistry 2022, 94, 7383-7390. https://doi.org/10.1021/acs.analchem.2c00870.
(5) Kelly, K. L.; et al. The Optical Properties of Metal Nanoparticles: The Influence of Size, Shape, and Dielectric Environment. J. Phys. Chem. B 2003, 107 (3), 668-677. https://doi.org/10.1021/jp026731y.
(6) Jain, P. K.; et al. Review of Some Interesting Surface Plasmon Resonance-Enhanced Properties of Noble Metal Nanoparticles and Their Applications to Biosystems. Plasmonics 2007, 2, 107-118. https://doi.org/10.1007/s11468-007-9031-1.
(7) Kimling, J.; et al. Turkevich Method for Gold Nanoparticle Synthesis Revisited. Journal of Physical Chemistry B 2006, 110 (32), 15700-15707. https://doi.org/10.1021/jp061667w.
(8) Jin, Z.; et al. A Charge-Switchable Zwitterionic Peptide for Rapid Detection of SARS-CoV-2 Main Protease. Angewandte Chemie International Edition 2022, 61. https://doi.org/10.1002/anie.202112995.
(9) Sun, J.; et al. Colorimetric Sensor Array Based on Gold Nanoparticles: Design Principles and Recent Advances. TrAC—Trends in Analytical Chemistry 2020, 122, 115754. https://doi.org/10.1016/j.trac.2019.115754.
(10) Hu, T.; et al. Colorimetric Sandwich Immunosensor for Aβ(1-42) Based on Dual Antibody-Modified Gold Nanoparticles. Sensors and Actuators, B: Chemical 2017, 243, 792-799. https://doi.org/10.1016/j.snb.2016.12.052.
(11) Retout, M.; et al. Rapid and Selective Detection of Proteins by Dual Trapping Using Gold Nanoparticles Functionalized with Peptide Aptamers. ACS Sensors 2016, 1, 929-933. https://doi.org/10.1021/acssensors.6b00229.
(12) Vilela, D.; et al. Sensing Colorimetric Approaches Based on Gold and Silver Nanoparticles Aggregation: Chemical Creativity behind the Assay. A Review. Analytica Chimica Acta 2012, 751, 24-43. https://doi.org/10.1016/j.aca.2012.08.043.
(13) He, H.; et al. Unusual Sequence Length-Dependent Gold Nanoparticles Aggregation of the SsDNA Sticky End and Its Application for Enzyme-Free and Signal Amplified Colorimetric DNA Detection. Sci. Rep. 2016, 6 (24), 1-7. https://doi.org/10.1038/srep30878.
(14) Lin, S.-Y.; et al. A Simple Strategy for Prompt Visual Sensing by Gold Nanoparticles: General Applications of Interparticle Hydrogen Bonds. Angewandte Chemie 2006, 118 (30), 5070-5073. https://doi.org/10.1002/ange.200600771.
(15) Hamaker, H. C. The London-van Der Waals Attraction between Spherical Particles. Physica 1937, 4 (10), 1058-1072. https://doi.org/10.1016/50031-8914(37)80203-7.
(16) Retout, M.; et al. Peptide-Induced Fractal Assembly of Silver Nanoparticles for Visual Detection of Disease Biomarkers. ACS Nano 2022. https://doi.org/10.1021/acsnano.1c11643.
(17) Charbgoo, F.; et al. Gold Nanoparticle Should Understand Protein Corona for Being a Clinical Nanomaterial. Journal of Controlled Release 2018, 272 (October 2017), 39-53. https://doi.org/10.1016/j.jconrel.2018.01.002.
(18) Liang, X.; et al. Colorimetric Detection of Melamine in Complex Matrices Based on Cysteamine-Modified Gold Nanoparticles. Analyst 2011, 136 (1), 179-183. https://doi.org/10.1039/c0an00432d.
(19) Retout, M.; et al. Peptide-Conjugated Silver Nanoparticles for the Colorimetric Detection of the Oncoprotein Mdm2 in Human Serum. Chempluschem 2022, 1-8. https://doi.org/10.1002/cplu.202200043.
(20) Lin, L.; et al. Simultaneous Colorimetric Detection of a Variety of Salmonella Spp. in Food and Environmental Samples by Optical Biosensing Using Oligonucleotide-Gold Nanoparticles. 2019. https://doi.org/10.3389/fmicb.2019.01138.
(21) Derjaguin, V. The DERJAGUIN-LANDAU-VERWEY-OVERBEEK (DLVO) Theory Stability of Lyophobic Colloids. 1987, No. 1986, 293-294.
(22) Mani, E.; et al. Equilibrium and Non-Equilibrium Cluster Phases in Colloids with Competing Interactions. Soft Matter 2014, 10 (25), 4479-4486. https://doi.org/10.1039/c3sm53058b.
(23) Liu, D.; et al. Resettable, Multi-Readout Logic Gates Based on Controllably Reversible Aggregation of Gold Nanoparticles. Angewandte Chemie 2011, 123 (18), 4189-4193. https://doi.org/10.1002/ange.201008198.
(24) He, H.; et al. Light-Induced Reversible Self-Assembly of Gold Nanoparticles Surface-Immobilized with Coumarin Ligands. Angewandte Chemie 2016, 128 (3), 948-952. https://doi.org/10.1002/ange.201508355.
(25) Hazarika, P.; Ceyhan, B.; Niemeyer, C. M. Reversible Switching of DNA-Gold Nanoparticle Aggregation. Angewandte Chemie 2004, 116 (47), 6631-6633. https://doi.org/10.1002/ange.200461887.
(26) Trantakis, I. A.; et al. Reversible Aggregation of DNA-Decorated Gold Nanoparticles Controlled by Molecular Recognition. Langmuir 2013, 29 (34), 10824-10830. https://doi.org/10.1021/1a401211u.
(27) Retout, Maurice; et al. Synthesis of Ultra-Stable and Bioconjugable Ag, Au and Bimetallic Ag_Au Nanoparticles Coated with Calix[4]Arenes. ACS Omega 2021, 6, 19675-19684.
(28) Retout, M.; et al. Bifunctional Calix[4]Arene-Coated Gold Nanoparticles for Orthogonal Conjugation. Langmuir 2022, 38, 9301-9309.
(29) Moore, C.; et al. Multispectral Nanoparticle Tracking Analysis for the Real-Time and Label-Free Characterization of Amyloid-β Self-Assembly in Vitro. Analytical Chemistry 2020, 92 (17), 11590-11599. https://doi.org/10.1021/acs.analchem.0c01048.
(30) Retout, M.; et al. Ultrastable PEGylated Calixarene-Coated Gold Nanoparticles with a Tunable Bioconjugation Density for Biosensing Applications. Bioconjugate Chem. 2021, 32, 290-300. https://doi.org/10.1021/acs.bioconjchem.0c00669.
(31) Smith, M. C.; et al. Quantitative Analysis of PEG-Functionalized Colloidal Gold Nanoparticles Using Charged Aerosol Detection. Analytical and Bioanalytical Chemistry 2015, 407, 3705-3716. https://doi.org/10.1007/s00216-015-8589-2.
(32) Jokerst, J. et al. Nanoparticle PEGylation for Imaging and Therapy. Nanomedicine 2011, 6, 715-728. https://doi.org/10.2217/nnm.11.19.
(33) Retout, M.; et al. Limits of Thiol Chemistry Revealed by Quantitative Analysis of Mixed Layers of Thiolated-PEG Ligands Grafted onto Gold Nanoparticles. Journal of Colloid and Interface Science 2019, 557, 807-815. https://doi.org/10.1016/j.jcis.2019.09.047.
(34) Ghann, W.; et al. Lipoic Acid Decorated Gold Nanoparticles and Their Application in the Detection of Lead Ions. Journal of Nanomedicine & Nanotechnology Research Article 1 J Nanomed Nanotechnol 2019, 10 (5), 539. https://doi.org/10.35248/2157-7439.19.10.539.
(35) Fracasso, D.; et al. Self-Assembled Monolayers of Terminal Acetylenes as Replacements for Thiols in Bottom-up Tunneling Junctions. RSC Adv. 2014, 4, 56026-56030. https://doi.org/10.1039/C4RA09880C.
(36) Zhdanov, V. P. Nanoparticles without and with Protein Corona: Van Der Waals and Hydration Interaction. Journal of Biological Physics 2019, 45 (3), 307-316. https://doi.org/10.1007/s10867-019-09530-8.
(37) de La Llave, E.; et al. Binding between Carbon and the Au(111) Surface and What Makes It Different from the S—Au(111) Bond. Journal of Physical Chemistry C 2008, 112 (45), 17611-17617. https://doi.org/10.1021/jp8036395.
(38) Yang, J.; et al. DeepDigest: Prediction of Protein Proteolytic Digestion with Deep Learning. Analytical Chemistry 2021, 93 (15), 6094-6103. https://doi.org/10.1021/acs.analchem.0c04704.
(39) Guo, H.; et al. Near-Infrared Fluorescent and Magnetic Resonance Dual-Imaging Coacervate Nanoprobes for Trypsin Mapping and Targeted Payload Delivery of Malignant Tumors. ACS Applied Materials and Interfaces 2020, 12 (15), 17302-17313. https://doi.org/10.1021/acsami.0c03433.
(40) Moore, C.; et al. Activatable Carbocyanine Dimers for Photoacoustic and Fluorescent Detection of Protease Activity. ACS Sensors 2021, 6 (6), 2356-2365. https://doi.org/10.1021/acssensors.1c00518.
(41) de Souza Nascimento, A. M.; et al. Antibacterial Action Mechanisms and Mode of Trypsin Inhibitors: A Systematic Review. Journal of Enzyme Inhibition and Medicinal Chemistry. Taylor and Francis Ltd. 2022, pp 749-759. https://doi.org/10.1080/14756366.2022.2039918.

### REFERENCES EXAMPLE 2

1 Liu, J. & Lu, Y. A colorimetric lead biosensor using DNAzyme-directed assembly of gold nanoparticles. Journal of the American Chemical Society 125, 6642-6643 (2003).
2 Jin, Z. et al. Peptide Amphiphile Mediated Co-assembly for Nanoplasmonic Sensing. Angewandte Chemie 135, e202214394 (2023).
3 Guo, L., et al. Oriented gold nanoparticle aggregation for colorimetric sensors with surprisingly high analytical figures of merit. Journal of the American Chemical Society 135, 12338-12345 (2013).
4 Liu, J. & Lu, Y. Accelerated color change of gold nanoparticles assembled by DNAzymes for simple and fast colorimetric Pb2+ detection. Journal of the American Chemical Society 126, 12298-12305 (2004).
5 Ghosh, S. K. et al. Interparticle coupling effect on the surface plasmon resonance of gold nanoparticles: from theory to applications. Chemical reviews 107, 4797-4862 (2007).
6 Jin, Z. et al. A Charge-Switchable Zwitterionic Peptide for Rapid Detection of SARS-CoV-2 Main Protease. Angewandte Chemie International Edition 61, e202112995 (2022).
7 Jokerst, J. V. et al. Peptide valence-induced breaks in plasmonic coupling. Chemical Science (2023).
8 Kim, T., et al. Control of gold nanoparticle aggregates by manipulation of interparticle interaction. Langmuir 21, 9524-9528 (2005).
9 Lin, S. Y., et al. A simple strategy for prompt visual sensing by gold nanoparticles: general applications of interparticle hydrogen bonds. Angewandte Chemie International Edition 45, 4948-4951 (2006).
10 Zhang, L., Mazouzi, Y., Salmain, M., Liedberg, B. & Boujday, S. Antibody-gold nanoparticle bioconjugates for biosensors: synthesis, characterization and selected applications. Biosensors and Bioelectronics 165, 112370 (2020).
11 Wang, Y. et al. Enhanced dispersion stability of gold nanoparticles by the physisorption of cyclic poly (ethylene glycol). Nature communications 11, 6089 (2020).
12 Yim, W. et al. Enhanced Photoacoustic Detection of Heparin in Whole Blood via Melanin Nanocapsules Carrying Molecular Agents. ACS nano 16, 683-693 (2021).
13 Cedervall, T. et al. Understanding the nanoparticle-protein corona using methods to quantify exchange rates and affinities of proteins for nanoparticles. Proceedings of the National Academy of Sciences 104, 2050-2055 (2007).
14 Retout, M. et al. Di-Arginine Additives for Dissociation of Gold Nanoparticle Aggregates: A Matrix-Insensitive Approach with Applications in Protease Detection. ACS Applied Materials & Interfaces 14, 52553-52565 (2022).
15 Liang, X. et al. Colorimetric detection of melamine in complex matrices based on cysteamine-modified gold nanoparticles. Analyst 136, 179-183 (2011).
16 Jokerst, J. V., Lobovkina, T., Zare, R. N. & Gambhir, S. S. Nanoparticle PEGylation for imaging and therapy. Nanomedicine 6, 715-728 (2011).
17 Jin, Z. et al. Structure of Mpro from SARS-CoV-2 and discovery of its inhibitors. Nature 582, 289-293 (2020).
18 Owen, D. R. et al. An oral SARS-CoV-2 Mpro inhibitor clinical candidate for the treatment of COVID-19. Science 374, 1586-1593 (2021).
19 Yim, W. et al. 3D-Bioprinted Phantom with Human Skin Phototypes for Biomedical Optics. Advanced Materials 35, 2206385 (2023).
20 Zhu, G., Zhu, X., Fan, Q. & Wan, X. Raman spectra of amino acids and their aqueous solutions. Spectrochimica Acta Part A: Molecular and Biomolecular Spectroscopy 78, 1187-1195 (2011).
21 Levy, R. et al. Rational and combinatorial design of peptide capping ligands for gold nanoparticles. Journal of the American Chemical Society 126, 10076-10084 (2004).
22 Nowinski, A. K., et al. Sequence, structure, and function of peptide self-assembled monolayers. Journal of the American Chemical Society 134, 6000-6005 (2012).
23 Mokashi-Punekar, S., et al. Tuning the structure and chiroptical properties of gold nanoparticle single helices via peptide sequence variation. Journal of the American Chemical Society 141, 15710-15716 (2019).
24 Liu, X., et al. Rational design of functional peptide-gold hybrid nanomaterials for molecular interactions. Advanced Materials 32, 2000866 (2020).
25 Olmedo, I. et al. How changes in the sequence of the peptide CLPFFD-NH2 can modify the conjugation and stability of gold nanoparticles and their affinity for β-amyloid fibrils. Bioconjugate chemistry 19, 1154-1163 (2008).
26 Jin, Z. et al. Spacer Matters: All-Peptide-Based Ligand for Promoting Interfacial Proteolysis and Plasmonic Coupling. Nano Letters 22, 8932-8940 (2022).
27 Yim, W. et al. Gold nanorod-melanin hybrids for enhanced and prolonged photoacoustic imaging in the near-infrared-II window. ACS applied materials & interfaces 13, 14974-14984 (2021).
28 Chen, S., et al. Ultra-low fouling peptide surfaces derived from natural amino acids. Biomaterials 30, 5892-5896 (2009).
29 YináYi, M. An AM1 and PM3 molecular orbital and self-consistent reaction-field study of the aqueous solvation of glycine, alanine and proline in their neutral and zwitterionic forms. Journal of the Chemical Society, Perkin Transactions 2, 531-537 (1991).
30 Huang, N. et al. SARS-CoV-2 infection of the oral cavity and saliva. Nature medicine 27, 892-903 (2021).
31 Retout, M. et al. Peptide-Conjugated Silver Nanoparticles for the Colorimetric Detection of the Oncoprotein Mdm2 in Human Serum. ChemPlusChem 87, e202100450 (2022).
32 Shan, Y. F. et al. Study on substrate specificity at subsites for severe acute respiratory syndrome coronavirus 3CL protease. Acta biochimica et biophysica Sinica 37, 807-813 (2005).
33 Kimling, J. et al. Turkevich method for gold nanoparticle synthesis revisited. The Journal of Physical Chemistry B 110, 15700-15707 (2006).
34 Salomon-Ferrer, R., et al. An overview of the Amber biomolecular simulation package. Wiley Interdisciplinary Reviews: Computational Molecular Science 3, 198-210 (2013).
35 Mark, P. et al. Structure and dynamics of the TIP3P, SPC, and SPC/E water models at 298 K. Journal ofPhysical Chemistry A 105, 9954-9960 (2001).
36 Ackland, G. J., et al. Simple N-body potentials for the noble metals and nickel. Philosophical Magazine A 56, 735-756 (1987).
37 Ryckaert, J.-P., et al. Numerical integration of the cartesian equations of motion of a system with constraints: molecular dynamics of n-alkanes. Journal of computational physics 23, 327-341 (1977).
38 Plimpton, S. Fast Parallel Algorithms for Short-Range Molecular Dynamics. Journal of Computational Physics 117, 1-19 (1995).

A number of embodiments of the invention have been described. Nevertheless, it can be understood that various modifications may be made without departing from the spirit and scope of the invention. Accordingly, other embodiments are within the scope of the following claims.

1: A product of manufacture, formulation, mixture or kit for: stabilizing or substantially stabilizing nanoparticles in a reversible aggregate, and, detecting the presence of a protease in a fluid,
the products of manufacture, formulations, mixtures or kits comprising:
(a) a compound X capable of making or forming into reversibly aggregated (or substantially reversibly aggregated) nanoparticles (NPs),
wherein the compound X comprises:
a nanoparticle (NP) aggregated with or stabilized with (Arginine)ₓ (or Argₓ or Rₓ), or an Argₓ citrate-stabilized nanoparticle, or a plurality of Argₓ citrate-stabilized nanoparticles (NPs), or Argₓ-NPs, wherein x is an integer 2, 3, 4, 5 or 6, and optionally the Rₓ comprises AA, AAA, AAAA (SEQ ID NO:1), AAAAA (SEQ ID NO:2) or AAAAAA (SEQ ID NO:3),
a nanoparticle (NP) aggregated with or stabilized with Lysineₓ-Rₓ (or -Lysₓ-Rₓ, or Kₓ-Rₓ, or an Kₓ-Rₓ citrate-stabilized nanoparticle, or a plurality of Kₓ-Rₓ citrate-stabilized nanoparticles (NPs), or Kₓ-Rₓ-NPs, wherein x is an integer 1, 2, 3, 4, 5 or 6,
and compound X aggregates (or is capable of aggregating, or substantially aggregates) when in a liquid solution,
(b) a compound Z conjugated to a poly(ethylene glycol) (Z-PEGₓ), or a plurality of poly(ethylene glycol) (Z-PEGₓs), wherein X is an integer 1, 2, 3, 4, 5, 6, 7, 8, 9, 10, 11, 12,13,14 or 15, or X is an integer between about 1 and 40, or between about 2 and 30, or compound Z is conjugated to a peptide,
wherein compound Z conjugated to:
(i) a poly(ethylene glycol) (Z-PEGₓ), or a plurality of poly(ethylene glycol) (Z-PEGₓs) is capable of dissociating compound X aggregates (for example, Arg-Arg-NPs aggregates) in a liquid solution,
wherein each Z-PEGₓ is conjugated to a protease peptide substrate or an amino acid sequence specifically recognized and cleaved by a protease by a compound Y comprising a moiety or a functional group linking the Z-PEGₓ to the protease peptide substrate (or Z-PEGₓ-Y-protease peptide substrate), or
(ii) a peptide.

2: The product of manufacture, formulation, mixture or kit of claim 1, wherein the protease is a viral or a mammalian protease.

3: The product of manufacture, formulation, mixture or kit of claim 1, wherein the fluid comprises or is derived from: (a) a biological fluid, optionally a biological fluid from an in vivo source, optionally an undiluted and/or untreated biological fluid from an in vivo source, and optionally the biological fluid from an in vivo source comprises blood, plasma, saliva, urine, bile, a lacrimal duct solution or tear, or cerebrospinal fluid (CSF); (b) a cell lysate; or (c) water, distilled water, saline or sea water.

4: The product of manufacture, formulation, mixture or kit of claim 1, wherein the citrate-stabilized NPs is about 20 nm in diameter, or is between about 10 and 50 nm in diameter.

5: The product of manufacture, formulation, mixture or kit of claim 1, wherein the citrate-stabilized NPs is prepared using a Turkevich method comprising:
(a) rapidly injecting an aqueous solution of sodium citrate tribasic dihydrate (SCTD) (optionally 150 mg SCTD, 5 mL aqueous solution) into an aqueous solution of HAuCl₄·3H₂O (optionally 45 mg HAuCl₄·3H₂O, 300 mL aqueous solution) under boiling conditions and vigorous stirring, to produce a reaction mixture; and
(b) the reaction mixture is left boiling while stirring for another about 15 min and then cooled down to room temperature (RT) to generate a deep red dispersion, and the deep red dispersion was then purified by applying one round of centrifugation at about 18,000 g for about 30 min to generate a pellet of AuNPs-citrate, and the pink supernatant is discarded, and the resulting pellet of AuNPs-citrate is redispersed in deionized water by sonication and stored at ambient conditions.

6: A solution or formulation comprising the plurality of Arg-Arg-NPs of claim 1(a) and the plurality of Z-PEGₓ-Y-peptide of claim 1(b), and optionally the solution or formulation comprises a saline solution, water, a cell lysate or a biological solution, and optionally the biological solution comprises a biological fluid from an in vivo source comprises a cell lysate solution, blood, plasma, saliva, urine, bile, a lacrimal duct solution or tear, or cerebrospinal fluid (CSF).

7: A method for detecting a protease in a sample, comprising: mixing the Arg-Arg-NPs of claim 1(a) and the Z-PEGₓ-Y-peptide of claim 1(b) in a sample, and presence of a protease capable of specifically recognizing and cleaving the peptide is detected via a proteolytic cleavage that releases Z-PEGₓ fragments from a Z-PEG-Y-peptide, thus inducing the dissociation of the AuNPs-citrate assemblies and turning the solution from blue to red.

8: The method of claim 7, wherein the sample comprises a cell lysate or a biological solution.

9: The product of manufacture, formulation, mixture or kit of claim 1, wherein the products formulations or mixtures can aggregate or assemble into nanoparticles such that the nanoparticles undergo plasmonic coupling, and the plasmonic coupling is reversible, or substantially reversible, leading to monodisperse nanoparticles when a chemical cue or signal is added.

10: The product of manufacture, formulation, mixture or kit of claim 1, wherein the NP comprises a di-arginine (Arg) citrate-stabilized nanoparticle, or a plurality of di-arginine (Arg) citrate-stabilized nanoparticles (NPs), or Arg-Arg-NPs.

11: The product of manufacture, formulation, mixture or kit of claim 1, wherein the Kₓ-Rₓ comprises KR, RK, RRK, KRR, RKR, RRKR (SEQ ID NO:4), RKRR (SEQ ID NO:5), KRRR (SEQ ID NO:6), RRRK (SEQ ID NO:7), KKRR (SEQ ID NO:8), KKKR (SEQ ID NO:9), RKKK (SEQ ID NO:10), KRRK (SEQ ID NO:11) or RKKR (SEQ ID NO:12).

12: The product of manufacture, formulation, mixture or kit of claim 1, wherein the biological fluid is derived or isolated from an in vivo source.

13: The product of manufacture, formulation, mixture or kit of claim 12, wherein the biological fluid is an undiluted and/or untreated biological fluid from an in vivo source.

14: The product of manufacture, formulation, mixture or kit of claim 12, wherein the biological fluid from an in vivo source comprises blood, plasma, saliva, urine, bile, a lacrimal duct solution or a tear, or cerebrospinal fluid (CSF); a cell lysate; or water, distilled water, saline or sea water,

15: The product of manufacture, formulation, mixture or kit of claim 1, wherein compound X, or the citrate-stabilized compound X, or the citrate-stabilized nanoparticle (NP) comprises or is conjugated to a metal to generate a metal-nanoparticle or metal-compound X.

16: The product of manufacture, formulation, mixture or kit of claim 15, wherein the metal of the metal nanoparticle or metal-compound X comprises silver (Ag) (for example, the nanoparticle comprises Arg-Arg-Ag-NP) or gold (Au) (for example, the nanoparticle comprises Arg-Arg-Au-NP).

17: The product of manufacture, formulation, mixture or kit of claim 1, wherein:
(a) each peptide comprises two three four, five, six, seven, eight, nine, ten, eleven or twelve or more amino acids, and optionally the peptide comprises EEKKPPC (SEQ ID NO:18),
(b) one way of coupling Z to the nanoparticle comprises use of thiol on a Cys moiety,
(c) the peptide comprises one or more amino acids with a negative charge at the opposite end that is bound to the nanoparticle where one or more of these negative amino acids are acetylated to increase negative charge,
(d) each peptide comprises one or more spacer amino acids including glycine, proline or alanine that increase the distance between the binding amino acid and the charged amino acid,
(e) each peptide comprises EEKKPPC (SEQ ID NO:18), where the cysteine (or C) binds to a gold (Au) surface, E (glutamic acid) has a negative charge and is acetylated, K (lysine) is positively charged, and P (proline) adds steric bulk,
(f) the compound Z comprises:
a thiol to generate a thiolated (HS) poly(ethylene glycol) (HS-PEGₓ), or a plurality of thiolated (HS) poly(ethylene glycol) (HS-PEGₓs),
an alkyne, to generate a poly(ethylene glycol) (ALK-PEGₓ), or a plurality of poly(ethylene glycol) (ALK-PEGₓs),
a lipoic acid group, to generate a poly(ethylene glycol) (LIP-PEGₓ), or a plurality of poly(ethylene glycol) (LIP-PEGₓs),
(g) the compound Y comprises:
a carboxyl group (for formation of an amide bond between the PEGₓ and peptide),
an azido group (for formation of a copper (I)-catalyzed alkyne-azide cycloaddition (CuAAC) to chemically join the PEGₓ and peptide),
an alkyne group (for formation of a copper (I)-catalyzed alkyne-azide cycloaddition (CuAAC) to chemically join the PEGₓ and peptide), or
a maleimide group (to generate a Michael reaction addition thiol group to chemically join the PEGₓ and peptide).
(h) each polyethylene glycol (PEG) moiety, or x, comprises one, two three four, five, six, seven, eight, nine, ten, eleven or twelve or more PEG repetitions,
(i) each Z-PEGₓ is conjugated to a protease peptide substrate (or an amino acid sequence specifically recognized and cleaved by a protease) (Z-PEGₓ-Y-peptide), and/or
(j) a plurality of compound X make or form into reversibly aggregated nanoparticles (NPs) when in the liquid solution, and optionally can stay in a substantially aggregated state indefinitely before being resuspended back to a state of monodispersity by the addition of compound Z to the liquid solution.

18: The product of manufacture, formulation, mixture or kit of claim 2, wherein the viral protease is a coronavirus protease.

19: The product of manufacture, formulation, mixture or kit of claim 18, wherein the coronavirus protease is a Covid-19 protease or SARS-CoV-2 (Mpro).

20: The method of claim 8, wherein the biological solution comprises a biological fluid from an in vivo source comprises a cell lysate solution, blood, plasma, a lacrimal duct solution or (a tear), saliva, urine, bile or cerebrospinal fluid (CSF); or the sample comprises water or a saline solution.


SEQUENCE LISTING (47 sequences)

SEQ ID NO: 1 - protein, 4 aa, synthetic construct
  REGION 1..4: note = Synthetic
AAAA

SEQ ID NO: 2 - protein, 5 aa, synthetic construct
  REGION 1..5: note = Synthetic
AAAAA

SEQ ID NO: 3 - protein, 6 aa, synthetic construct
  REGION 1..6: note = Synthetic
AAAAAA

SEQ ID NO: 4 - protein, 4 aa, synthetic construct
  REGION 1..4: note = Synthetic
RRKR

SEQ ID NO: 5 - protein, 4 aa, synthetic construct
  REGION 1..4: note = Synthetic
RKRR

SEQ ID NO: 6 - protein, 4 aa, synthetic construct
  REGION 1..4: note = Synthetic
KRRR

SEQ ID NO: 7 - protein, 4 aa, synthetic construct
  REGION 1..4: note = Synthetic
RRRK

SEQ ID NO: 8 - protein, 4 aa, synthetic construct
  REGION 1..4: note = Synthetic
KKRR

SEQ ID NO: 9 - protein, 4 aa, synthetic construct
  REGION 1..4: note = Synthetic
KKKR

SEQ ID NO: 10 - protein, 4 aa, synthetic construct
  REGION 1..4: note = Synthetic
RKKK

SEQ ID NO: 11 - protein, 4 aa, synthetic construct
  REGION 1..4: note = Synthetic
KRRK

SEQ ID NO: 12 - protein, 4 aa, synthetic construct
  REGION 1..4: note = Synthetic
RKKR

SEQ ID NO: 13 - protein, 6 aa, synthetic construct
  REGION 1..6: note = Synthetic
RRKRRK

SEQ ID NO: 14 - protein, 9 aa, synthetic construct
  REGION 1..9: note = Synthetic
RRKRRKRRK

SEQ ID NO: 15 - protein, 5 aa, synthetic construct
  REGION 1..5: note = Synthetic
RRRRR

SEQ ID NO: 16 - protein, 5 aa, synthetic construct
  REGION 1..5: note = Synthetic
RGGGR

SEQ ID NO: 17 - protein, 12 aa, synthetic construct
  REGION 1..12: note = Synthetic
GTSAVLQSGFRK

SEQ ID NO: 18 - protein, 7 aa, synthetic construct
  REGION 1..7: note = Synthetic
EEKKPPC

SEQ ID NO: 19 - protein, 4 aa, synthetic construct
  REGION 1..4: note = Synthetic
KKEE

SEQ ID NO: 20 - protein, 4 aa, synthetic construct
  REGION 1..4: note = Synthetic
EEKC

SEQ ID NO: 21 - protein, 8 aa, synthetic construct
  REGION 1..8: note = Synthetic
KEEKKPPC

SEQ ID NO: 22 - protein, 7 aa, synthetic construct
  REGION 1..7: note = Synthetic
EEKKAAC

SEQ ID NO: 23 - protein, 7 aa, synthetic construct
  REGION 1..7: note = Synthetic
EEKKGGC

SEQ ID NO: 24 - protein, 9 aa, synthetic construct
  REGION 1..9: note = Synthetic
EEKKGGGGC

SEQ ID NO: 25 - protein, 11 aa, synthetic construct
  REGION 1..11: note = Synthetic
EEKKGGGGGGC

SEQ ID NO: 26 - protein, 7 aa, synthetic construct
  REGION 1..7: note = Synthetic
CGGKKEE

SEQ ID NO: 27 - protein, 14 aa, synthetic construct
  REGION 1..14: note = Synthetic
CGGKKEEAVLQSGF

SEQ ID NO: 28 - protein, 4 aa, synthetic construct
  REGION 1..4: note = Synthetic
AVLQ

SEQ ID NO: 29 - protein, 7 aa, synthetic construct
  REGION 1..7: note = Synthetic
AVLQSGF

SEQ ID NO: 30 - protein, 15 aa, synthetic construct
  REGION 1..15: note = Synthetic
CGGKKEEAVLQSGFR

SEQ ID NO: 31 - protein, 4 aa, synthetic construct
  REGION 1..4: note = Synthetic
SGFR

SEQ ID NO: 32 - protein, 7 aa, synthetic construct
  REGION 1..7: note = Synthetic
EEKKPPG

SEQ ID NO: 33 - protein, 8 aa, synthetic construct
  REGION 1..8: note = Synthetic
KEEPPKKC

SEQ ID NO: 34 - protein, 7 aa, synthetic construct
  REGION 1..7: note = Synthetic
KKEEPPC

SEQ ID NO: 35 - protein, 5 aa, synthetic construct
  REGION 1..5: note = Synthetic
EEPPC

SEQ ID NO: 36 - protein, 5 aa, synthetic construct
  REGION 1..5: note = Synthetic
KKPPC

SEQ ID NO: 37 - protein, 5 aa, synthetic construct
  REGION 1..5: note = Synthetic
EEKKC

SEQ ID NO: 38 - protein, 7 aa, synthetic construct
  REGION 1..7: note = Synthetic
EEKKAAG

SEQ ID NO: 39 - protein, 4 aa, synthetic construct
  REGION 1..4: note = Synthetic
GGGG

SEQ ID NO: 40 - protein, 7 aa, synthetic construct
  REGION 1..7: note = Synthetic
GGGGGGG

SEQ ID NO: 41 - protein, 9 aa, synthetic construct
  REGION 1..9: note = Synthetic
CGGKEAVLQ

SEQ ID NO: 42 - protein, 11 aa, synthetic construct
  REGION 1..11: note = Synthetic
EEKKGGCAVLQ

SEQ ID NO: 43 - protein, 11 aa, synthetic construct
  REGION 1..11: note = Synthetic
AVLQEEKKGGC

SEQ ID NO: 44 - protein, 9 aa, synthetic construct
  REGION 1..9: note = Synthetic
AVLQEKGGC

SEQ ID NO: 45 - protein, 11 aa, synthetic construct
  REGION 1..11: note = Synthetic
AVLQCGGKKEE

SEQ ID NO: 46 - protein, 10 aa, synthetic construct
  REGION 1..10: note = Synthetic
SGFEEKKGGC

SEQ ID NO: 47 - protein, 7 aa, synthetic construct
  REGION 1..7: note = Synthetic
EEPPKKC